(19) United States
(12) Patent Application Publication
(10) Pub. No.: US 2014/0187556 A1
(43) Pub. Date: Jul. 3, 2014

(54) ANTI-AMYLOID COMPOUNDS AND METHODS

(71) Applicants: Mark A. Reed, Hubley (CA); Arun Yadav, Halifax (CA); Scott C. Banfield, Ellershouse (CA); Christopher J. Barden, Lake Side (CA)

(72) Inventors: Mark A. Reed, Hubley (CA); Arun Yadav, Halifax (CA); Scott C. Banfield, Ellershouse (CA); Christopher J. Barden, Lake Side (CA)

(73) Assignee: Treventis Corporation, Bernville, PA (US)

(21) Appl. No.: 14/002,031

(22) PCT Filed: Mar. 2, 2012
(86) PCT No.: PCT/US12/27399

§ 371 Date: Mar. 3, 2014

Related U.S. Application Data
(60) Provisional application No. 61/448,969, filed on Mar. 3, 2011.

(51) Int. Cl. C07D 295/192; C07C 217/84; C07D 295/096; C07D 211/14; C07D 257/04

(52) U.S. Cl. CPC C07D 295/192; C07D 211/14; C07D 257/04; C07D 295/096; C07C 217/84
USPC 514/235.8; 514/252.11; 514/253.09; 514/254.01; 514/254.05; 514/255.01; 514/429; 514/647; 544/121; 544/357; 544/364; 544/366; 544/372; 544/391; 548/577; 564/307

(57) ABSTRACT

Anti-amyloid compounds are provided along with methods of use thereof.

### CROSS-REFERENCE TO RELATED APPLICATIONS

This application claims the benefit of U.S. Provisional Application Ser. No. 61/448,969, filed Mar. 3, 2011, the contents of which are hereby incorporated by reference in their entirety.

### BACKGROUND OF THE INVENTION

The build-up of amyloid proteins in living tissue, a condition known as amyloidosis, is either the cause or a major factor in the pathology of many so-called amyloid diseases such as Alzheimer's, Parkinson's, Huntington's, and prion diseases. Historically, aggregations of protein were classified as amyloid if they displayed apple-green birefringence under polarized light when stained with the dyes Congo red or Thioflavin T (ThT) (Sipe and Cohen, 2000, J. Struct. Biol. 130:88-98). That definition of amyloid has been expanded in recent years to apply to any polypeptide which can polymerize in a cross-3 sheet conformation in vitro or in vivo, regardless of sequence (Xu, 2007, Amyloid 14:119-31). Certain types of amyloidosis may occur principally in the central nervous system, as with aggregation of beta-amyloid protein in Alzheimer's Disease, alpha-synuclein in Parkinson's Disease, huntinigtin protein in Huntington's Disease, and prion protein in Creutzfeldt-Jacob and other prion diseases. Other types of amyloidosis are systemic in nature, as with aggregation of transthyretin in senile systemic amyloidosis.

All of the above listed diseases are invariably fatal using current medical practice. In none of these diseases is there any known, widely accepted therapy or treatment that can halt and/or reverse the aggregation of amyloid deposits. As such there remains an urgent need for treatments.

### OBJECTS AND SUMMARY OF THE INVENTION

It is an object of the present invention to provide new compounds useful in the treatment of amyloidosis.

It is an object of the present invention to provide methods useful in the treatment of amyloidosis.

It is another object of the present invention to provide methods for administering to a subject a therapeutic compound which inhibits amyloid aggregation.

It is another object of the present invention to provide pharmaceutical compositions for treating amyloidosis. The pharmaceutical compositions include a therapeutic compound of the invention in an amount effective to inhibit amyloid aggregation and a pharmaceutically acceptable excipient or vehicle.

In accordance with the above objects and others, the present invention provides methods and compounds which are useful in the treatment of amyloidosis. The methods of the invention involve administering to a subject a therapeutic compound which inhibits amyloid aggregation. Accordingly, the compounds and methods of the invention are useful for treating disorders in which amyloidosis occurs. The methods of the invention can be used therapeutically to treat amyloidosis or can be used prophylactically in a subject susceptible to amyloidosis.

The invention further provides pharmaceutical compositions for treating amyloidosis. The pharmaceutical compositions include a therapeutic compound of the invention in an amount effective to inhibit amyloid aggregation and a pharmaceutically acceptable vehicle. In certain preferred embodiments, the pharmaceutical composition is an oral solid dosage form (e.g., tablet or capsule), an oral liquid dosage form, or an injectable dosage form.

In accordance with the above, the present invention is directed in part to a compound of any of Formulas I:

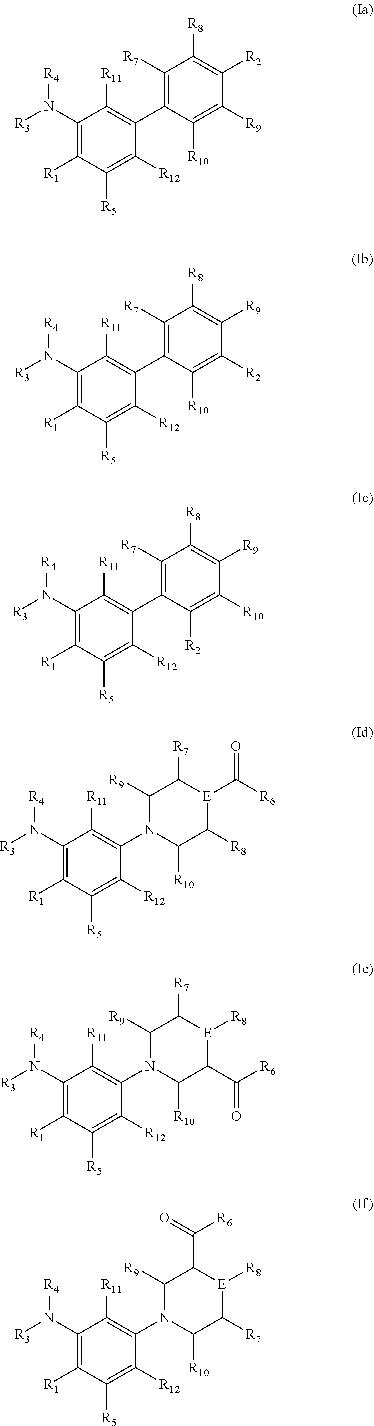

in which R₁ is nitro, difluoromethyl ketone, halogen, trifluoromethylsulfone, trifluoromethyl ether, difluoromethyl ether, hydrogen, trifluoromethyl, cyano, isopropylamine, or N-linked tetrazole; R₂, when present, is C-linked tetrazole, sulfonamide, alkylamide, dialkylamide, benzyl alkylamide, N-pyrrolidinamide, (N′-methanonylpiperazine)amide, (N′-methylpiperazine)amide, morphilinamide, piperidineamide, ethanol-1-yl, methanol, 2,2,2-trifluoro-1-hydroxyethanol-1-yl, 2,2,2-trifluoroethanol-1-yl, or cyano; R₃ is benzyl, isopropyl, ethyl, cyclopropyl, or cyclobutyl; R₄ is hydrogen, CH₂CH₂NCH₃ alkyl, 3-(N-pyrrolidinyl)propyl, propyl, or CH₂CH₂O; R₃ and R₄ are unconnected or connect to form piperizine, N-pyrrolidine, 4-(N-pyrrolidinyl)piperidine, morpoline, or piperidine rings; R₅ is hydrogen, halogen, or trifluoromethyl; R₆, when present, is alkyl, N-pyrollidine, alkylamine, dialkylamine, or phenyl optionally substituted with alkyl, halogen, or alkoxy independently at each open position; E, when present, is carbon or nitrogen; and R₇, R₈, R₉, R₁₀, R₁₁, and R₁₂ are each independently hydrogen or alkyl.

In certain preferred embodiments, the compound is according to Formula Is a. In certain preferred embodiments, the compound is according to Formula Ib. In certain preferred embodiments, the compound is according to Formula Ic. In certain preferred embodiments, the compound is according to Formula Id. In certain preferred embodiments, the compound is according to Formula Ie. In certain preferred embodiments, the compound is according to Formula If. In certain preferred embodiments, E is carbon. In certain other preferred embodiments, E is nitrogen. In certain preferred embodiments, R₁ is nitro and/or R₂ is selected from the group consisting of ethanol-1-yl, methanol, 2,2,2-trifluoro-1-hydroxyethanol-1-yl, and 2,2,2-trifluorocthanol-1-yl. In certain preferred embodiments, the compound is 1-(3′-(benzylamino)-4′-nitro-[1,1′-biphenyl]-3-yl)-2,2,2-trifluoroethanol. In certain preferred embodiments, the compound is 1-(3′-(benzylamino)-4′-nitro-[1,1′-biphenyl]-3-yl)-2,2,2-trifluoroethane-1,1-diol. In certain preferred embodiments, R₁ is nitro and/or R₂ is C-linked tetrazole. In certain preferred embodiments, R₁ is nitro. In certain preferred embodiments, R₂ is selected from the group consisting of sulfonamide, alkylamide, dialkylamide, benzyl alkylamide, N-pyrrolidinamide, (N′-methanonylpiperazine)amide, (N′-methylpiperazine)amide, morphilinamide, and piperidineamide.

The compounds of the present invention inhibit the aggregation of an amyloidogenic protein. The amyloid disease may be, e.g., Alzheimer's disease, Parkinson's disease, Huntington's disease, or prion disease.

In another aspect, the present invention is directed in part to a pharmaceutical composition having a compound of any one of the preceding claims and a pharmaceutically acceptable excipient. Such a composition could be in oral or parenteral dosage form, for example.

In another aspect, the invention is directed in part to a compound selected from the group consisting of those compounds identified herein by TRV 1093 to TRV 1192 inclusive.

In another aspect, the invention is directed in part to a method of treatment for an amyloid disease is provided including administering a therapeutically effective dose of a compound described herein to a subject in need thereof.

In another aspect, the invention is directed to pharmaceutical compositions comprising an effective amount of one or more of a compound of any of Formulas I together with other active agents, e.g., other compounds for treating amyloidosis. In additional aspects, such embodiments are also directed to the use of such pharmaceutical compositions comprising an effective amount of one or more of a compound of any of Formulas I together with other active agents, e.g., other compounds for treating amyloidosis, for the treatment of amyloidosis.

In accordance with the above, the present invention is also directed to pharmaceutically acceptable salts, stereoisomers, polymorphs, metabolites, analogues, and pro-drugs of the compounds of Formulas Ia, Ib, Ic, Id, Ie, and If, and any combination thereof.

The term “subject” is intended to include living organisms in which amyloidosis can occur. Examples of subjects include humans, monkeys, cows, sheep, goats, dogs, cats, mice, rats, and transgenic species thereof.

### BRIEF DESCRIPTION OF DRAWING

FIG. 1 shows the result of a ThS tau aggregation assay (fluorescence vs time) for previously disclosed compounds compared to compounds of the present invention with respect to control aggregation of Tau441 in DMSO; lower fluorescence is better.

### DETAILED DESCRIPTION

The therapeutic compounds of the invention are administered to a subject by a route which is effective for inhibition of amyloid aggregation. Suitable routes of administration include subcutaneous, intravenous and intraperitoneal injection. A preferred route of administration is oral administration. The therapeutic compounds may be administered with a pharmaceutically acceptable vehicle.

This invention pertains to methods and compositions useful for treating amyloidosis. The methods of the invention involve administering to a subject a therapeutic compound which inhibits amyloid aggregation. “Inhibition of amyloid aggregation” is intended to encompass prevention of amyloid deposition, inhibition of further amyloid deposition in a subject with ongoing amyloidosis, and reduction of amyloid deposits in a subject with ongoing amyloidosis. Inhibition of amyloid aggregation is determined relative to an untreated subject or relative to the treated subject prior to treatment. Amyloid aggregation is inhibited by interfering with the binding of monomeric and/or oligomeric amyloid protein to other, nearby amyloid protein such that aggregation of amyloid is inhibited. This inhibition of amyloid aggregation may have effects on both chain and step polymerization mechanisms of amyloid proteins, and may affect the aggregation of both heterogeneous and homogeneous amyloid deposits. Examples of amyloid proteins include, but are by no means limited to, beta-amyloid protein, tau protein, alpha-synuclein protein, immunoglobulin light chain protein, insulin, Islet amyloid polypeptide, lysozyme, transthyretin, amyloid A, prion protein, and polyglutamate (huntingtin) protein.

In certain embodiments, one or more of the compounds in the invention may be combined at concentrations or dosages discussed above with a pharmaceutically or pharmacologically acceptable carrier, excipient or diluent, either biodegradable or non-biodegradable. Examples of exemplary examples of carriers include, but are by no means limited to, for example, poly(ethylene-vinyl acetate), copolymers of lactic acid and glycolic acid, poly(lactic acid), gelatin, collagen matrices, polysaccharides, poly(D,L lactide), poly(malic acid), poly(caprolactone), celluloses, albumin, starch, casein, dextran, polyesters, ethanol, mathacrylate, polyurethane, polyethylene, vinyl polymers, glycols, mixtures thereof and the like. Standard excipients include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, polyvinylpyrrolidone, sugars and starches.

As will be apparent to one knowledgeable in the art, specific carriers and carrier combinations known in the art may be selected based on their properties and release characteristics in view of the intended use. Specifically, the carrier may be pH-sensitive, thermo-sensitive, thermo-gelling, arranged for sustained release or a quick burst. In some embodiments, carriers of different classes may be used in combination for multiple effects, for example, a quick burst followed by sustained release.

In other embodiments, one or more of the compounds in the invention at concentrations or dosages described above may be encapsulated for delivery. Specifically, the compounds may be encapsulated in biodegradable microspheres, microcapsules, microparticles, or nanospheres. The delivery vehicles may be composed of, for example, hyaluronic acid, polyethylene glycol, poly(lactic acid), gelatin, poly(E-caprolactone), or a poly(lactic-glycolic) acid polymer. Combinations may also be used, as, for example, gelatin nanospheres may be coated with a polymer of poly(lactic-glycolic) acid. As will be apparent to one knowledgeable in the art, these and other suitable delivery vehicles may be prepared according to protocols known in the art and utilized for delivery of the compounds. It is of note that the compounds in the invention may be combined with permeation enhancers known in the art for improving delivery.

Patients amenable to treatment with the compounds of the present invention (e.g., a compound of any of Formulas I) include individuals at risk of disease but not showing symptoms, as well as patients presently showing symptoms. In the case of amyloidosis, virtually anyone is at risk of suffering from amyloidosis. Therefore, the present methods can be administered prophylactically to the general population without the need for any assessment of the risk of the subject patient. Such prophylactic administration can begin at, e.g., age 50 or greater. The present methods are especially useful for individuals who do have a known genetic risk of amyloidosis.

In prophylactic applications, pharmaceutical compositions containing one or more of the compounds of the present invention (e.g., a compound of any of Formulas I) are administered to a patient susceptible to, or otherwise at risk of, amyloidosis in an amount sufficient to eliminate or reduce the risk, lessen the severity, or delay the outset of the disease, including biochemical, histologic and/or behavioral symptoms of the disease, its complications and intermediate pathological phenotypes presented during development of the disease. In therapeutic applications, compositions or medicaments are administered to a patient suspected of, or already suffering from, such a disease in an amount sufficient to cure, or at least partially arrest, the symptoms of the disease biochemical, histologic and/or behavioral), including its complications and intermediate pathological phenotypes in development of the disease. In some methods, administration of the compound may reduce or eliminates mild cognitive impairment in patients that have not yet developed characteristic amyloidosis pathology. An amount adequate to accomplish therapeutic or prophylactic treatment is defined as a therapeutically- or prophylactically-effective dose.

A “prophylactically effective amount” of a disclosed compound in accordance with the present invention refers to an amount effective, at dosages and for periods of time necessary, to achieve the desired prophylactic result, such as preventing or inhibiting amyloidosis. A prophylactically effective amount can be determined as described above for the therapeutically effective amount. Typically, since a prophylactic dose is used in subjects prior to or at an earlier stage of disease, the prophylactically effective amount will be less than the therapeutically effective amount. A “therapeutically effective amount” refers to an amount effective, at dosages and for periods of time necessary, to achieve the desired therapeutic rest, such as slowed progression of amyloidosis, delayed onset, reduction or reversal of aggregate formation and/or neurofibrillary tangles, and/or reduction or reversal of neurotoxicity. A therapeutically effective amount of the compound(s) of the invention may vary according to factors such as the disease state, age, sex, and weight of the individual, and the ability of the modulator to elicit a desired response in the individual. Dosage regimens may be adjusted to provide the optimum therapeutic response. A therapeutically effective amount is also one in which any toxic or detrimental effects of the modulator are outweighed by the therapeutically beneficial effects.

Effective doses of the compositions of the present invention (e.g., a compound of any of Formulas I), for the treatment of the above described conditions vary depending upon many different factors, including means of administration, target site, physiological state of the patient, other medications administered, and whether treatment is prophylactic or therapeutic.

Pharmaceutical formulations in accordance with the present invention may comprise (i) one or more of the compounds of formula I disclosed herein and (ii) one or more pharmaceutically acceptable excipients. The active agent will generally comprise from about 0.01% to about 90% of the formulation, and the one or more excipients will generally comprise from about 10% to about 99.99% of the formulation. In the preferred embodiments, the formulations are used for introduction of the active agent into a body of a living mammal (e.g., a human).

The pharmaceutical compositions comprising the compounds of the present invention can be administered by parenteral, topical, intranasal, intravenous, oral, subcutaneous, intraarterial, intracranial, intraperitoneal, intranasal, or intramuscular means for prophylactic and/or therapeutic treatment. The pharmaceutical compositions in accordance with the present invention may also contain one or more pharmaceutical carriers and/or suitable adjuvants. They can also be combined where desired with other active agents, e.g., other compounds for treating amyloidosis. The pharmaceutical composition can be administered subcutaneously, intravenously, intradermally, intramuscularly, intraperitoneally, intracerebrally, intranasally, orally, transdermally, buccally, intra-arterially, intracranially, or intracephalically. It is especially advantageous to formulate parenteral compositions in dosage unit form for ease administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals. As used herein “pharmaceutically acceptable carrier” includes any and all solvents, dispersion media, coatings, antibacterial and antifungal agents, isotonic and absorption delaying agents, and the like that are physiologically compatible. In one embodiment, the carrier is suitable for parenteral administration. Preferably, the carrier can be suitable for intravenous, intraperitoneal or intramuscular administration. Alternatively, the carrier is suitable for administration into the central nervous system (e.g., intraspinally or intracerebrally). In another embodiment, the carrier is suitable for oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Injectable formulations prepared in accordance with the present invention typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the antibody can be administered in a time-release formulation, for example in a composition which includes a slow release polymer. The compounds of the present invention can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions comprising one or more of the compounds of the present invention can be prepared by incorporating the compound in an appropriate solvent with one or a combination of ingredients enumerated above, as required, followed by filtered sterilization.

For parenteral administration, the pharmaceutical compositions of the present invention can be administered as injectable dosages of a solution or suspension of the substance in a physiologically acceptable diluent with a pharmaceutical carrier that can be a sterile liquid such as water, oil, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin. Peanut oil, soybean oil, and mineral oil are all examples of useful materials. In general, glycols, such as propylene glycol or polyethylene glycol, are preferred liquid carriers, particularly for injectable solutions. Agents of the invention, particularly, antibodies, can be administered in the form of a depot injection or implant preparation which can be formulated in such a manner as to permit a sustained release of the active ingredient. An exemplary composition comprises monoclonal antibody at 5 mg/mL, formulated in aqueous buffer consisting of 50 mM L-histidine, 150 mM NaCl, adjusted to pH 6.0 with HCl.

Oral pharmaceutical compositions comprising an effective amount of one or more of the compounds of the present invention (e.g., compounds of any of Formulas I) can be suitably prepared with auxiliary agents, e.g., lubricants, preservatives, stabilizers, wetting agents, emulsifiers, salts for influencing osmotic pressure buffers, coloring, flavoring and/or aromatic substances and the like. They can also be combined where desired with other active agents, e.g., other compounds for treating amyloidosis. The compositions intended for oral use (e.g., tablets, capsules, etc.) may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay release of the active ingredients. Formulations for oral use may also be presented as hard gelatin capsules wherein the active ingredient is mixed with an inert diluent.

In certain preferred embodiments of the present invention, a tablet core is obtained by mixing the required quantity of one or more of the compounds of any of Formulas I having a necessary particle size with other materials usually included in tablets, such as diluents, lubricants, binders, etc. In certain embodiments, for example, it may be necessary to include one or more disintegrants in the tablet core. After the insoluble drug is mixed with the additional tableting ingredients, the mixture is then tableted on a suitable tableting machine.

Aqueous suspensions contain the above-identified combination of drugs and that mixture has one or more excipients suitable as suspending agents, for example pharmaceutically acceptable synthetic gums such as hydroxypropylmethylcellulose or natural gums. Oily suspensions may be formulated by suspending the above-identified combination of drugs in a vegetable oil or mineral oil. The oily suspensions may contain a thickening agent such as beeswax or cetyl alcohol. A syrup, elixir, or the like can be used wherein a sweetened vehicle is employed. Injectable suspensions may also be prepared, in which case appropriate liquid carriers, suspending agents and the like may be employed. It is also possible to freeze-dry the active compounds and use the obtained lyophilized compounds, for example, for the preparation of products for injection.

Topical application can result from intransdermal or intradermal application. Topical administration can be facilitated by coadministration of the agent with cholera toxin or detoxified derivatives or subunits thereof. Alternatively, transdermal delivery can be achieved using skin patch or using transfersomes.

Other delivery systems can include time-release, delayed release or sustained release delivery systems. Such systems can avoid repeated administrations of the active compounds of the invention, increasing convenience to the subject and the physician. Many types of release delivery systems are available and known to those of ordinary skill in the art. They include polymer based systems such as polylactic and polyglycolic acids polyanhydrides and polycaprolactone; nonpolymer systems that are lipids including sterols such as cholesterol, cholesterol esters and fatty acids or neutral fats such as mono-, di and triglycerides; hydrogel release systems; silastic systems; peptide based systems; wax coatings, compressed tablets using conventional binders and excipients, partially fused implants and the like. In addition, a pump-based hardware delivery system can be used, some of which are adapted for implantation.

A long-term sustained release implant also may be used. “Long-term” release, as used herein, means that the implant is constructed and arranged to deliver therapeutic levels of the active ingredient for at least 30 days, and preferably 60 days. Long-term sustained release implants are well known to those of ordinary skill in the art and include some of the release systems described above. Such implants can be particularly useful in treating conditions characterized by aggregates of amyloid beta peptides by placing the implant near portions of the brain affected by such aggregates, thereby effecting localized, high doses of the compounds of the invention.

In the methods of the invention, amyloid aggregation in a subject is inhibited by administering a therapeutic compound of the invention to the subject. The term subject is intended to include living organisms in which amyloidosis can occur. Examples of subjects include humans, monkeys, cows, sheep, goats, dogs, cats, mice, rats, and transgenic species thereof. Administration of the compositions of the present invention to a subject to be treated can be carried out using known procedures, at dosages and for periods of time effective to inhibit amyloid aggregation in the subject. An effective amount of the therapeutic compound necessary to achieve a therapeutic effect may vary according to factors such as the amount of amyloid already deposited at the clinical site in the subject, the age, sex, and weight of the subject, and the ability of the therapeutic compound to inhibit amyloid aggregation in the subject. Dosage regimens can be adjusted to provide the optimum therapeutic response. For example, several divided doses can be administered daily or the dose may be proportionally reduced as indicated by the exigencies of the therapeutic situation. A non-limiting example of an effective dose range for a therapeutic compound of the invention is between 0.05 and 500 mg/kg of body weight per day. As a non-limiting example, the compounds in the invention may be arranged to be delivered at a concentration of about 100 nM to about 5 mM; or 1 μM to about 5′ mM; or 10 μM to 5 mM; or 100 μM to 5 mM. As will be appreciated by one of skill in the art, this may be the effective concentration, that is, a sufficient dosage is administered such that a concentration within one of the envisioned ranges is attained at the required site. In preferred embodiments, the brain/plasma ratio of the compound is above about 0.5, more preferably above about 1.

Active compounds are administered at a therapeutically effective dosage sufficient to inhibit amyloid aggregation in a subject. A “therapeutically effective dosage” preferably inhibits amyloid aggregation by at least about 20%, more preferably by at least about 40%, even more preferably by at least about 60%, and still more preferably by at least about 80% relative to untreated subjects. The ability of a compound to inhibit amyloid aggregation can be evaluated in an animal model system that may be predictive of efficacy in inhibiting amyloid aggregation in human diseases. Alternatively, the ability of a compound to inhibit amyloid aggregation can be evaluated by examining the ability of the compound to inhibit the aggregation of an amyloid protein in a binding assay, e.g. the ThT assay described in the Embodiments.

Compounds referred to by identifiers that are denoted “TRV XXXX” where XXXX is a number are synonymous with those identified as “TRV-XXXX” and/or “BCW XXXX” where XXXX is the same number.

By “alkyl”, we mean any cyclic, branched, or unbranched aliphatic moiety of up to six carbons; examples include methyl, ethyl, propyl, butyl, isopropyl, cyclopropyl, sec-butyl, cyclopentyl, pentyl, neopentyl, cyclohexyl, and 2-methylpropyl.

By “halogen”, we mean fluorine, chlorine, or bromine.

By “alkoxy”, we mean any alkyl ether; examples include methoxy and ethoxy.

By “open position” with respect to a phenyl moiety, we mean positions which are not connected to other moieties in the compound and which would, when unsubstituted, carry hydrogens.

When two R groups are said to be “connected to form” a ring, we mean that given two R groups R_{X} and R_{Y} that are already connected to each other via at least one non-hydrogenic atom belonging to neither group, a non-hydrogenic atom in R_{X} is covalently bonded to a non-hydrogenic atom in R_{Y} such that a ring is formed by making this inter-R-group bond. For example, when R_{X} and R_{Y} are both ethyl and are both connected to a nitrogen atom belong to neither group, ring species that could result include N-pyrrolidine and 2-methyl-N-aziridine.

For moieties that contain chiral centers, for example:

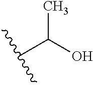 [SMILES: *C(C)O]

the compound containing such a moiety may be used as enantiomerically pure (R or S) or as a racemate; or in some enantiomeric excess in between, for example, between 90 and 99% e.e., or between 80% and 99%, or between 65% and 99%, or between 50% and 99%.

The invention is further illustrated by the following examples which should not be construed as further limiting the subject invention. The contents of all references and published patent applications cited throughout this application are hereby incorporated by reference. A demonstration of efficacy of the therapeutic compounds of the present invention in the ThT assay is predictive of efficacy in humans. Unless otherwise mentioned, terms and abbreviations used below are meant to have their meaning as understood by a practitioner skilled in the art.

### DETAILED DESCRIPTION OF PREFERRED EMBODIMENTS

The invention is further illustrated by the following examples which should not be construed to further limited the present invention in any manner. The contents of all references and published patents cited throughout this application are hereby incorporated by reference. A demonstration of efficacy of the therapeutic compounds of the present invention in the ThT assay is predicative of efficacy in humans. Unless otherwise mentioned, terms and abbreviations used below are meant to have their meaning as understood by a practitioner skilled in the art.

### Example 1

### Determination of anti-amyloid potency for Aβ using the Thioflavin T (ThT) aggregation assay

The following methodologies were used:

Preparation of Aβ₄₀ Stock Solutions

Aβ₄₀ (1.0 mg) was pre-treated in a 1.5 mL microfuge tube with HFIP (1 mL) and sonicated for 20 min to disassemble any pre-formed Aβ aggregates. The HFIP was removed with a stream of argon and the Aβ dissolved in Tris base (5.8 mL, 20 mM, pH ˜10). The pH was adjusted to 7.4 with concentrated HCl (−10 μL) and the solution filtered using a syringe filter (0.2 μm) before being used.

ThT Aβ Aggregation Assay

The kinetic ThT assay for Aβ aggregation is similar to that of Chalifour et al (Chalifour et al, 2003, J. Biol. Chem. 278:34874-81). Briefly, pre-treated Aβ40 (40 μM in 20 mM Tris, pH 7.4), was diluted with an equal volume of 8 μM ThT in Tris (20 mM, pH 7.4, 300 mM NaCl). Aliquots of Aβ/ThT (200 μL) were added to wells of a black polystyrene 96-well plate, followed by 2 μL of a compound in DMSO (variable concentration), or DMSO alone (controls). Incubations were performed in triplicate and were taken to contain 20 μM Aβ, various concentration of compound in 20 mM Tris, pH 7.4, 150 mM NaCl, 1% DMSO. Plates were covered with clear polystyrene lids and incubated at 37 degrees C. in a Tecan Genios microplate reader. Fluorescence readings (λex=450 nm, λem=480 nm) were taken every 15 min., after first shaking at high intensity for 15 s and allowing to settle for 10 s before each reading. Active compounds attenuated the increase in fluorescence over time that occurred in controls.

ThT results for selected compounds of the present invention are shown in the following table, along with results from the experiment of Example 4 where available:

| | | | Stability in |
|---|---|---|---|
| | | | mouse liver |
| | | | microsomes |
| | | | (% test |
| ID (e.g. | | | compound |
| 1192 = | | | remaining |
| TRV | | ThT Aβ | after 60 |
| 1192) | Structure | IC₅₀ (μM) | min) |
| 1192 | 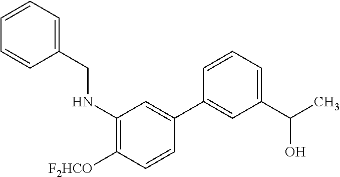 [SMILES: CC(O)c1cccc(-c2ccc(OC(F)F)c(NCc3ccccc3)c2)c1] | ~50 | |
| 1190 | 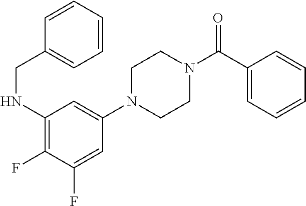 [SMILES: O=C(c1ccccc1)N1CCN(c2cc(F)c(F)c(NCc3ccccc3)c2)CC1] | ~50 | |
| 1186 | 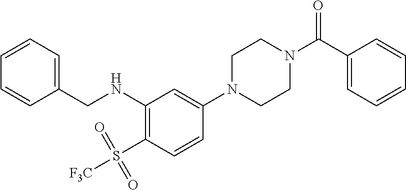 [SMILES: O=C(c1ccccc1)N1CCN(c2ccc(S(=O)(=O)C(F)(F)F)c(NCc3ccccc3)c2)CC1] | >50 | |
| 1185 | 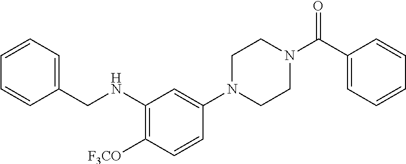 [SMILES: O=C(c1ccccc1)N1CCN(c2ccc(OC(F)(F)F)c(NCc3ccccc3)c2)CC1] | >50 | |
| 1184 | 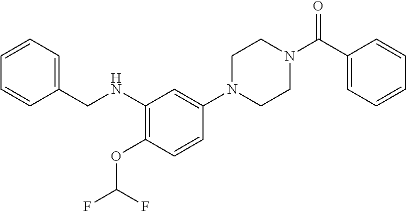 [SMILES: O=C(c1ccccc1)N1CCN(c2ccc(OC(F)F)c(NCc3ccccc3)c2)CC1] | >50 | |
| 1179 | 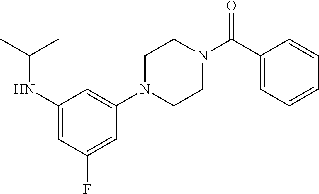 [SMILES: CC(C)Nc1cc(F)cc(N2CCN(C(=O)c3ccccc3)CC2)c1] | >50 | |
| 1178 | 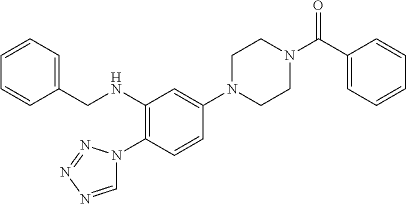 [SMILES: O=C(c1ccccc1)N1CCN(c2ccc(-n3cnnn3)c(NCc3ccccc3)c2)CC1] | >50 | |
| 1176 | 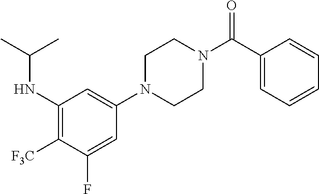 [SMILES: CC(C)Nc1cc(N2CCN(C(=O)c3ccccc3)CC2)cc(F)c1C(F)(F)F] | >50 | |
| 1175 | 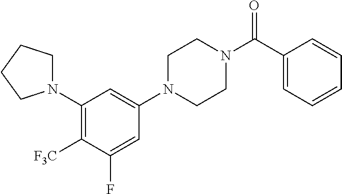 [SMILES: O=C(c1ccccc1)N1CCN(c2cc(F)c(C(F)(F)F)c(N3CCCC3)c2)CC1] | >50 | |
| 1170 | 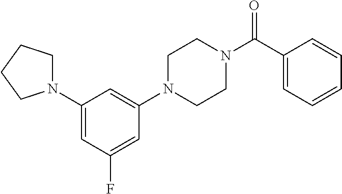 [SMILES: O=C(c1ccccc1)N1CCN(c2cc(F)cc(N3CCCC3)c2)CC1] | >50 | |
| 1169 | 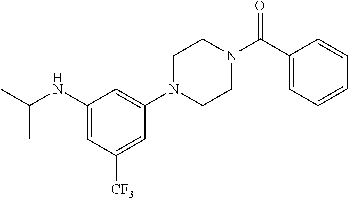 [SMILES: CC(C)Nc1cc(N2CCN(C(=O)c3ccccc3)CC2)cc(C(F)(F)F)c1] | >50 | |
| 1168 | 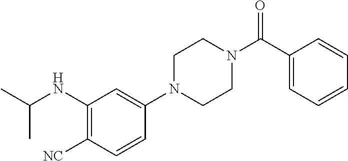 [SMILES: CC(C)Nc1cc(N2CCN(C(=O)c3ccccc3)CC2)ccc1C#N] | >50 | |
| 1167 | 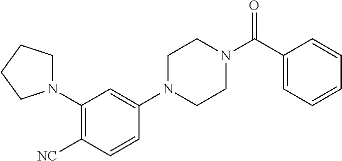 [SMILES: N#Cc1ccc(N2CCN(C(=O)c3ccccc3)CC2)cc1N1CCCC1] | >50 | |
| 1166 | 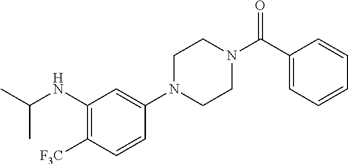 [SMILES: CC(C)Nc1cc(N2CCN(C(=O)c3ccccc3)CC2)ccc1C(F)(F)F] | >50 | |
| 1164 | 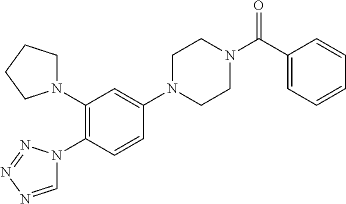 [SMILES: O=C(c1ccccc1)N1CCN(c2ccc(-n3cnnn3)c(N3CCCC3)c2)CC1] | >50 | |
| 1163 | 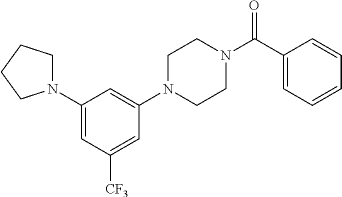 [SMILES: O=C(c1ccccc1)N1CCN(c2cc(N3CCCC3)cc(C(F)(F)F)c2)CC1] | >50 | |
| 1162 | 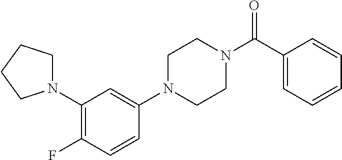 [SMILES: O=C(c1ccccc1)N1CCN(c2ccc(F)c(N3CCCC3)c2)CC1] | >50 | |
| 1160 | 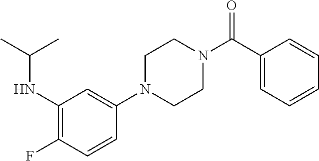 [SMILES: CC(C)Nc1cc(N2CCN(C(=O)c3ccccc3)CC2)ccc1F] | >50 | |
| 1159 | 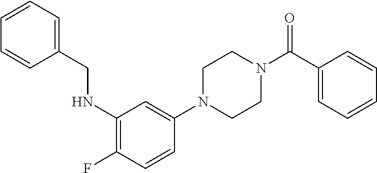 [SMILES: O=C(c1ccccc1)N1CCN(c2ccc(F)c(NCc3ccccc3)c2)CC1] | >50 | |
| 1158 | 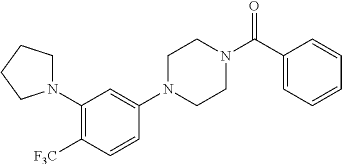 [SMILES: O=C(c1ccccc1)N1CCN(c2ccc(C(F)(F)F)c(N3CCCC3)c2)CC1] | 42.02 ± 0.12 | |
| 1157 | 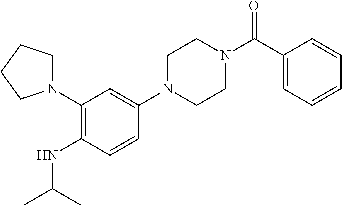 [SMILES: CC(C)Nc1ccc(N2CCN(C(=O)c3ccccc3)CC2)cc1N1CCCC1] | >50 | |
| 1156 | 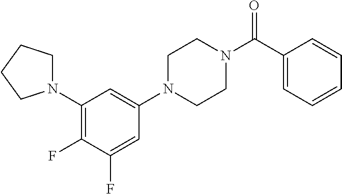 [SMILES: O=C(c1ccccc1)N1CCN(c2cc(F)c(F)c(N3CCCC3)c2)CC1] | >50 | |
| 1155 | 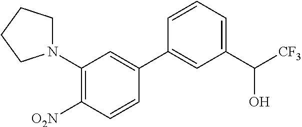 [SMILES: O=[N+]([O-])c1ccc(-c2cccc(C(O)C(F)(F)F)c2)cc1N1CCCC1] | 6.65 ± 1.97 | |
| 1154 | 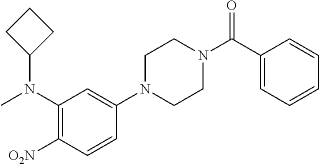 [SMILES: CN(c1cc(N2CCN(C(=O)c3ccccc3)CC2)ccc1[N+](=O)[O-])C1CCC1] | 11.61 ± 1.24 | |
| 1153 | 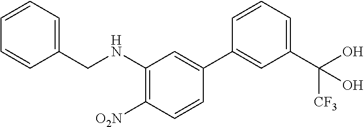 [SMILES: O=[N+]([O-])c1ccc(-c2cccc(C(O)(O)C(F)(F)F)c2)cc1NCc1ccccc1] | 7.08 ± 0.19 | |
| 1152 | 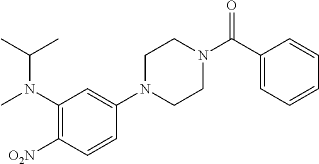 [SMILES: CC(C)N(C)c1cc(N2CCN(C(=O)c3ccccc3)CC2)ccc1[N+](=O)[O-]] | 16.88 ± 0.52 | |
| 1149 | 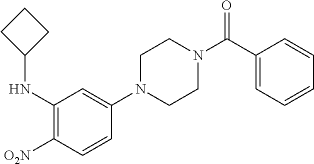 [SMILES: O=C(c1ccccc1)N1CCN(c2ccc([N+](=O)[O-])c(NC3CCC3)c2)CC1] | >10 | |
| 1147 | 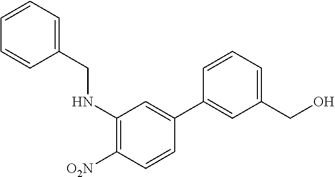 [SMILES: O=[N+]([O-])c1ccc(-c2cccc(CO)c2)cc1NCc1ccccc1] | 3.54 ± 0.46 | <1.0 |
| 1146 | 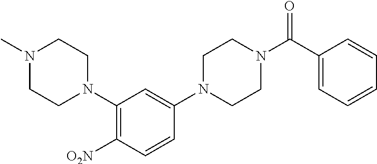 [SMILES: CN1CCN(c2cc(N3CCN(C(=O)c4ccccc4)CC3)ccc2[N+](=O)[O-])CC1] | >10 | |
| 1144 | 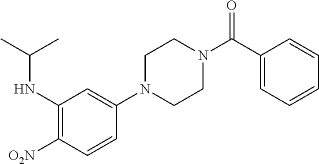 [SMILES: CC(C)Nc1cc(N2CCN(C(=O)c3ccccc3)CC2)ccc1[N+](=O)[O-]] | >10 | |
| 1143 | 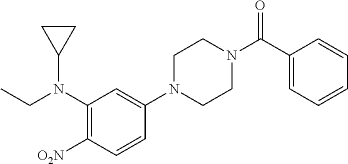 [SMILES: CCN(c1cc(N2CCN(C(=O)c3ccccc3)CC2)ccc1[N+](=O)[O-])C1CC1] | >10 | |
| 1142 | 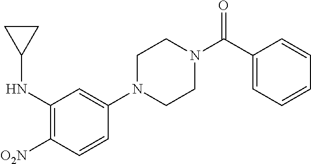 [SMILES: O=C(c1ccccc1)N1CCN(c2ccc([N+](=O)[O-])c(NC3CC3)c2)CC1] | ~10 | 1.7 |
| 1141 | 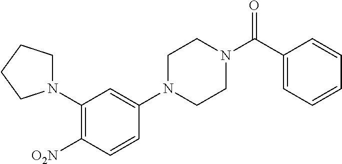 [SMILES: O=C(c1ccccc1)N1CCN(c2ccc([N+](=O)[O-])c(N3CCCC3)c2)CC1] | ~10 | <1.0 |
| 1140 | 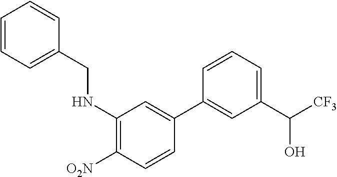 [SMILES: O=[N+]([O-])c1ccc(-c2cccc(C(O)C(F)(F)F)c2)cc1NCc1ccccc1] | 3.09 ± 0.21 | 34 |
| 1139 | 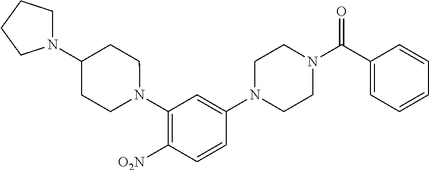 [SMILES: O=C(c1ccccc1)N1CCN(c2ccc([N+](=O)[O-])c(N3CCC(N4CCCC4)CC3)c2)CC1] | ~10 | |
| 1138 | 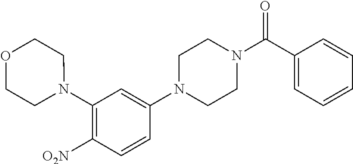 [SMILES: O=C(c1ccccc1)N1CCN(c2ccc([N+](=O)[O-])c(N3CCOCC3)c2)CC1] | >10 | |
| 1137 | 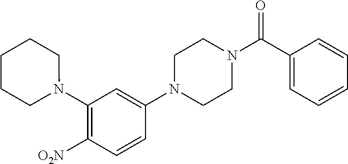 [SMILES: O=C(c1ccccc1)N1CCN(c2ccc([N+](=O)[O-])c(N3CCCCC3)c2)CC1] | ~10 | |
| 1136 | 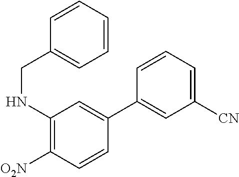 [SMILES: N#Cc1cccc(-c2ccc([N+](=O)[O-])c(NCc3ccccc3)c2)c1] | >50 | |
| 1135 | 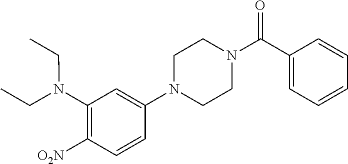 [SMILES: CCN(CC)c1cc(N2CCN(C(=O)c3ccccc3)CC2)ccc1[N+](=O)[O-]] | 12.37 ± 0.62 | |
| 1134 | 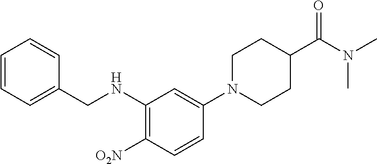 [SMILES: CN(C)C(=O)C1CCN(c2ccc([N+](=O)[O-])c(NCc3ccccc3)c2)CC1] | 10.70 ± 0.54 | |
| 1133 | 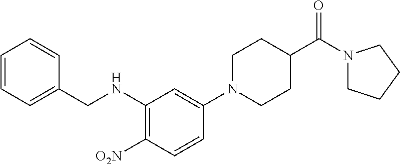 [SMILES: O=C(C1CCN(c2ccc([N+](=O)[O-])c(NCc3ccccc3)c2)CC1)N1CCCC1] | 17.88 ± 13.3 | |
| 1132 | 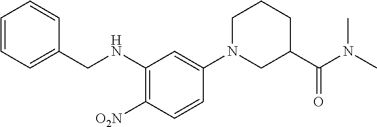 [SMILES: CN(C)C(=O)C1CCCN(c2ccc([N+](=O)[O-])c(NCc3ccccc3)c2)C1] | 11.5 ± 0.29 ± | |
| 1131 | 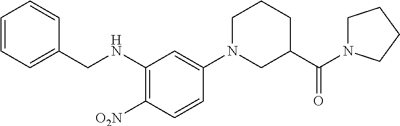 [SMILES: O=C(C1CCCN(c2ccc([N+](=O)[O-])c(NCc3ccccc3)c2)C1)N1CCCC1] | 27.8 ± 27.6 | |
| 1130 | 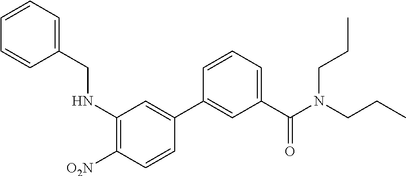 [SMILES: CCCN(CCC)C(=O)c1cccc(-c2ccc([N+](=O)[O-])c(NCc3ccccc3)c2)c1] | >50 | |
| 1129 | 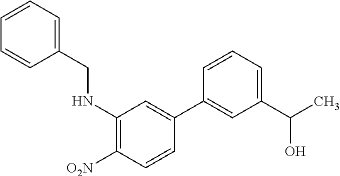 [SMILES: CC(O)c1cccc(-c2ccc([N+](=O)[O-])c(NCc3ccccc3)c2)c1] | 6.49 ± 0.21 | <1.0 |
| 1128 | 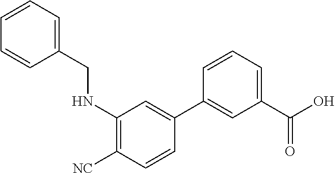 [SMILES: N#Cc1ccc(-c2cccc(C(=O)O)c2)cc1NCc1ccccc1] | >50 | |
| 1126 | 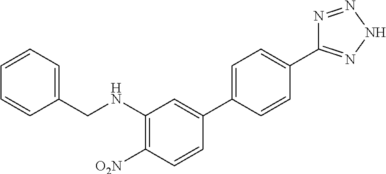 [SMILES: O=[N+]([O-])c1ccc(-c2ccc(-c3nn[nH]n3)cc2)cc1NCc1ccccc1] | 1.1 ± 0.33 | 41 |
| 1125 | 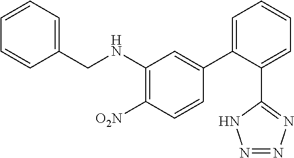 [SMILES: O=[N+]([O-])c1ccc(-c2ccccc2-c2nnn[nH]2)cc1NCc1ccccc1] | 12.5 ± 2.79 | |
| 1124 | 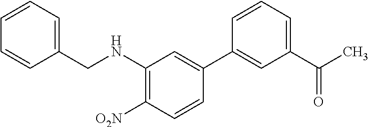 [SMILES: CC(=O)c1cccc(-c2ccc([N+](=O)[O-])c(NCc3ccccc3)c2)c1] | 6.49 ± 2.5 | |
| 1122 | 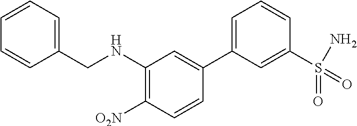 [SMILES: NS(=O)(=O)c1cccc(-c2ccc([N+](=O)[O-])c(NCc3ccccc3)c2)c1] | 14.14 ± 3.07 | 1.5 |
| 1121 | 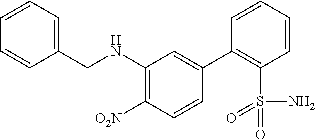 [SMILES: NS(=O)(=O)c1ccccc1-c1ccc([N+](=O)[O-])c(NCc2ccccc2)c1] | 24.57 ± 6.83 | <1.0 |
| 1120 | 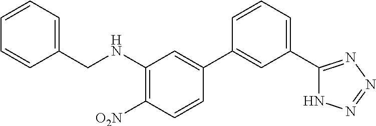 [SMILES: O=[N+]([O-])c1ccc(-c2cccc(-c3nnn[nH]3)c2)cc1NCc1ccccc1] | 1.34 ± 0.22 | 60 |
| 1118 | 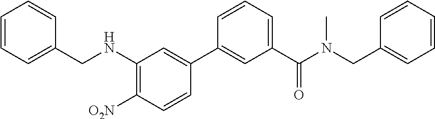 [SMILES: CN(Cc1ccccc1)C(=O)c1cccc(-c2ccc([N+](=O)[O-])c(NCc3ccccc3)c2)c1] | 6.47 ± 4.92 | |
| 1117 | 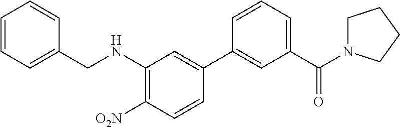 [SMILES: O=C(c1cccc(-c2ccc([N+](=O)[O-])c(NCc3ccccc3)c2)c1)N1CCCC1] | 2.94 ± 1.64 | <1.0 |
| 1116 | 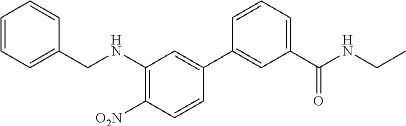 [SMILES: CCNC(=O)c1cccc(-c2ccc([N+](=O)[O-])c(NCc3ccccc3)c2)c1] | >50 | |
| 1115 | 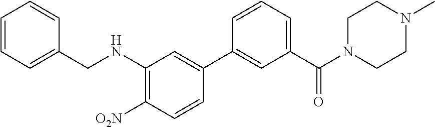 [SMILES: CN1CCN(C(=O)c2cccc(-c3ccc([N+](=O)[O-])c(NCc4ccccc4)c3)c2)CC1] | 2.67 ± 0.1 | <1.0 |
| 1114 | 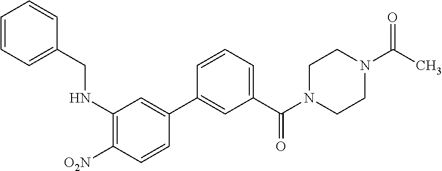 [SMILES: CC(=O)N1CCN(C(=O)c2cccc(-c3ccc([N+](=O)[O-])c(NCc4ccccc4)c3)c2)CC1] | >50 | |
| 1113 | 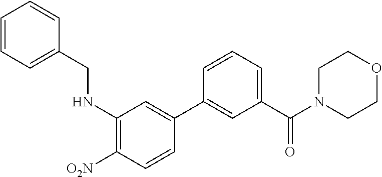 [SMILES: O=C(c1cccc(-c2ccc([N+](=O)[O-])c(NCc3ccccc3)c2)c1)N1CCOCC1] | >50 | |
| 1112 | 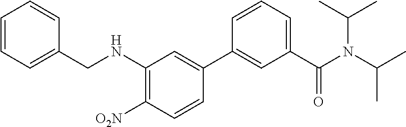 [SMILES: CC(C)N(C(=O)c1cccc(-c2ccc([N+](=O)[O-])c(NCc3ccccc3)c2)c1)C(C)C] | 1.41 ± 1.11 | <1.0 |
| 1111 | 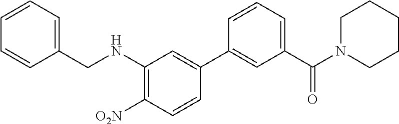 [SMILES: O=C(c1cccc(-c2ccc([N+](=O)[O-])c(NCc3ccccc3)c2)c1)N1CCCCC1] | 0.33 ± 0.44 | <1.0 |
| 1110 | 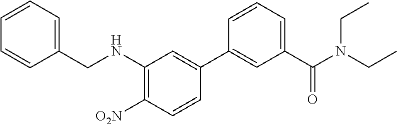 [SMILES: CCN(CC)C(=O)c1cccc(-c2ccc([N+](=O)[O-])c(NCc3ccccc3)c2)c1] | 0.70 ± 0.9 | <1.0 |
| 1103 | 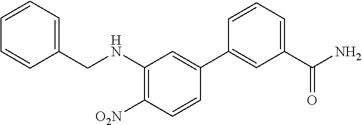 [SMILES: NC(=O)c1cccc(-c2ccc([N+](=O)[O-])c(NCc3ccccc3)c2)c1] | 10.45 ± 0.75 | |
| 1102 | 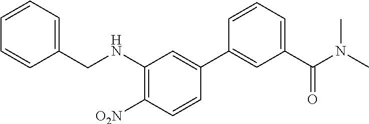 [SMILES: CN(C)C(=O)c1cccc(-c2ccc([N+](=O)[O-])c(NCc3ccccc3)c2)c1] | 4.11 ± 1.2 | <1.0 |
| 1101 | 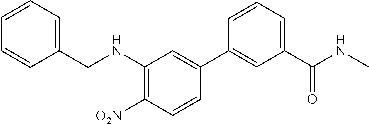 [SMILES: CNC(=O)c1cccc(-c2ccc([N+](=O)[O-])c(NCc3ccccc3)c2)c1] | >50 | |
| 1099 | 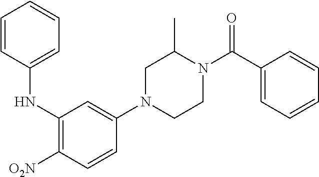 [SMILES: CC1CN(c2ccc([N+](=O)[O-])c(Nc3ccccc3)c2)CCN1C(=O)c1ccccc1] | 6.12 ± 2.78 | |
| 1098 | 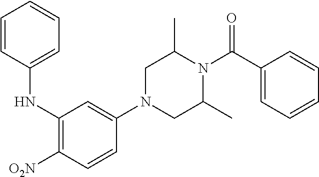 [SMILES: CC1CN(c2ccc([N+](=O)[O-])c(Nc3ccccc3)c2)CC(C)N1C(=O)c1ccccc1] | 3.99 ± 3.77 | |
| 1097 | 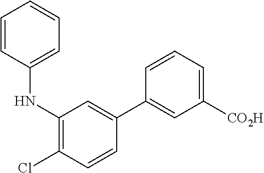 [SMILES: O=C(O)c1cccc(-c2ccc(Cl)c(Nc3ccccc3)c2)c1] | >50 | |
| 1096 | 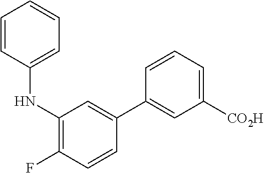 [SMILES: O=C(O)c1cccc(-c2ccc(F)c(Nc3ccccc3)c2)c1] | >50 | |
| 1095 | 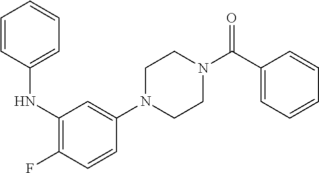 [SMILES: O=C(c1ccccc1)N1CCN(c2ccc(F)c(Nc3ccccc3)c2)CC1] | 15.95 ± 7.8 | <1.0 |
| 1094 | 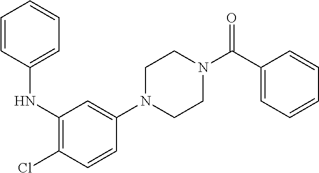 [SMILES: O=C(c1ccccc1)N1CCN(c2ccc(Cl)c(Nc3ccccc3)c2)CC1] | 31.6 ± 4.8 | |
| 1246 | 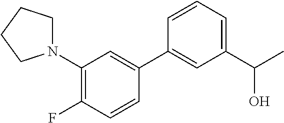 [SMILES: CC(O)c1cccc(-c2ccc(F)c(N3CCCC3)c2)c1] | 56.76% inhibition at 50 μM (IC₅₀ < 50 μM) | |
| 1248 | 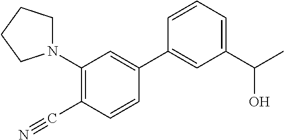 [SMILES: CC(O)c1cccc(-c2ccc(C#N)c(N3CCCC3)c2)c1] | 47.92% inhibition at 50 μM (IC₅₀~ 50 μM) | |

### Example 2

### Determination of Anti-Amyloid Potency for Tau Using the Thioflavin S (ThS) Aggregation Assay

Analogous to the method of Example 1, the ability of compounds to inhibit the aggregation of tau was performed by substituting AD with tau and Thioflavin T with Thioflavin S.

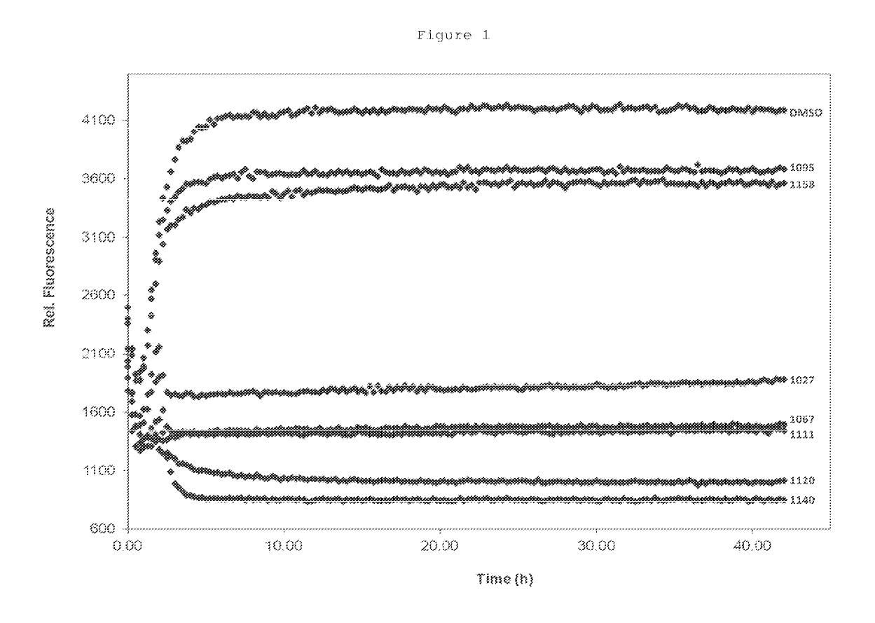

Results for this method are shown in FIG. 1 for selected compounds of the present invention as well as previously disclosed compounds TRV 1027 and TRV 1067. At 20 μM, TRV 1095 and TRV 1158 inhibit tau aggregation approximately 20%, which suggests their IC₅₀s are above 20 μM. The remainder of the compounds in FIG. 1 inhibit tau aggregation greater than 50%, which suggests their IC₅₀s are below 20 μM. Lower fluorescence is better; the extent to which each compound's IC₅₀ is below 20 μM is thus suggested by how low the fluorescence appears. Most potent is TRV 1140, followed by TRV 1120, followed by TRV 1111, followed by previously disclosed compound TRV 1067, followed by previously disclosed compound TRV 1027.

### Example 3

### Determination of Anti-Amyloid Potency for Alpha-Synuclein

Analogous to the method of Example 1, the ability of compounds to inhibit the aggregation of alpha-synuclein was performed by substituting Aβ with alpha-synuclein.

Results for this method indicate that compounds TRV 1120 and TRV 1140 each independently inhibit alpha-synuclein aggregation at 10 μM and may inhibit at less than 10 μM.

### Example 4

### Determination of Metabolic Stability in Mouse Liver Microsomes

This assay is used to determine the percent remaining and intrinsic clearance of a test compound incubated with pooled mouse liver microsomes in the presence of NADPH. Further details on this method may be found in Jeffrey P, et al., Utility of metabolic stability screening: comparison of in vitro and in vivo clearance. enobiotica. 2001 August-September; 31(8-9):591-8 and Lin J H, et al., Role of pharmacokinetics and metabolism in drug discovery and development. Pharmacol Rev. 1997; 49(4):403-49; each of which is hereby incorporated by reference. Test compound was formulated in 300 μL of DMSO at 10 mM and was diluted to 1 μM with final concentration of organic solvent <0.25% for the experiment. Pooled liver microsomes were prepared from male mice, with 1 mM NADPH. Test compound was incubated at 37 degrees Celsius in buffer containing 0.5 mg/mL microsomal protein; the reaction was initiated by adding cofactors and sampled at 0, 10, 20, 30, and 60 minutes. A positive control (5 μM testosterone) was incubated and initiated in parallel for quality control. Samples were analyzed using an LTQ-Orbitrap XL mass spectrometer; HRMS was used to determine the peak area response ratio (peak area corresponding to test compound or control divided by that of an analytical internal standard) without running a standard curve.

Results were reported for selected compounds in the table of Example 1, as % test compound remaining after 60 minutes. Compounds which have less than about 5% remaining are not considered to be metabolically stable in mouse liver microsomes, while compounds with more than about 5% remaining are considered to be metabolically stable in mouse liver microsomes.

### Example 5

### Prediction of Blood-Brain Barrier Penetration Potential Using MDR1-MDCK Cell Monolayers

This assay is used to determine the blood-brain barrier (BBB) penetration potential of a test compound using MDR1-MDCK cell monolayers. Further details on this method may be found in Taub M E, et al., Functional assessment of multiple Pglycoprotein (P-gp) probe substrates: Influence of cell line and modulator concentration on P-gp activity. Drug Metab Dispos. 2005 November; 33(11): 1679-87 and Wang Q, et al., Evaluation of the MDR-MDCK cell line as a permeability screen for the blood-brain barrier. Int J. Pharm. 2005 Jan. 20; 288(2):349-59; each of which is hereby incorporated by reference. Test compound was formulated in 300 μL of DMSO at 10 mM and diluted to 5 μM in HBSSg with maximum DMSO concentration not greater than 1%. Confluent monolayers of MDR1-MDCK cells, 7 to 11 days old, were used. A receiver well, having apical and basolateral sides at pH 7.4, was used with 1% BSA in modified Hanks buffer (HBSSg). Two monolayers were dosed with test compound in each direction (N=2). Apical side dosing was used for A-B assessment and basolateral side was used for B-A assessment; taken together, this information provides a bidirectional permeability potential. Apical and basolateral sides were sampled at 120 minutes and concentrations of test compound were determined using an LC-MS/MS method with a minimum 4 point calibration curve. Apparent permeability (Papp) in both directions (A-B and B-A) was calculated, as was the efflux ratio (Papp B-A)/(Papp A-B). Compounds were considered to have high BBB permeability if Papp (A-B) was greater than 3.0×10⁻⁶ cm/s and the efflux ratio was less than 3.0.

Results for selected test compounds and previously disclosed compound TRV 1067:

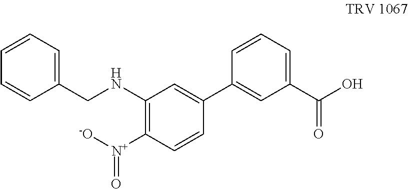

are presented in the table below:

| Test | | | Efflux |
|---|---|---|---|
| Compound ID | Pₐₚₚ A-B × 10⁻⁶ cm/s | Pₐₚₚ B-A × 10⁻⁶ cm/s | ratio |
| 1067 | 42.1 | 74.9 | 1.8 |
| 1120 | 38.3 | 47.4 | 1.2 |
| 1122 | 27.5 | 52.5 | 1.9 |
| 1126 | 34.1 | 47.8 | 1.4 |
| 1140 | 15.3 | 12.4 | 0.8 |

Thus, all compounds in the above table were predicted to have high BBB permeability.

### Example 6

### In Vivo Brain/Plasma Ratio Determination for Previously Disclosed Compound TRV 1067

Five groups of CD-1 mice (both sexes; individual weights 20 to 40 g) were dosed intraperitoneally with different doses of TRV 1067 (0, 3, 10, 30, and 100 mg/kg). DMSO (100%) was used as a vehicle. Animals had ad libitum access to rodent chow. Some animals were euthanized 2 hours after dosing on Day 1 and brains were harvested. Blood (0.15 mL) was collected 15 minutes after dosing and at sacrifice. The remaining animals were dosed once daily for 5 consecutive days. Blood samples were collected 15 minutes after dosing on Day 1. On Day 5, blood samples were collected 15 minutes and 2 hours after dosing and brains were harvested.

Standards were prepared in CD-1 mouse plasma containing sodium heparin as an anticoagulant and CD-1 mouse brain homogenate. An eight-point calibration curve was prepared at concentrations of 1000, 500, 100, 50, 10, 5, 1, and 0.5 ng/mL by serial dilution. Standard samples were treated identically to the study samples. Brain samples were homogenized with a Virsonic 100 ultrasonic homogenizer. Each brain sample was first weighed, and then an appropriate volume of 20:80 methanol:water was added to make a 4 mL/1 g sample. Samples were then homogenized on ice, and stored frozen until analysis. Plasma and brain homogenate samples were extracted via acetonitrile precipitation on a Tomtec Quadra 96-Model 320 liquid handling system in a 96-well plate format. A Perkin Elmer series 200 micropumps and LEAP Autosampler high-performance liquid chromatograph (HPLC) was used with a 0.2% formic acid/water/acetonitrile solvent system for separation purposes. The compound was quantitated using a PE Sciex API3000 mass spectrometer connected to the HPLC; negative polarity was used, with TRV 1067 analyzed using ion settings of 347.1 mass/charge ratio for the precursor and 212.1 for the product ions, respectively. Warfarin was used in negative polarity (307.1 precursor, 250.0 product) as an internal standard.

Results: Mean brain/plasma ratios for male CD-1 mice after 2 hours on Day 1 ranged from too low to determine at 3 mg/kg through 0.168 at 100 mg/kg. A similar measurement for female CD-1 mice ranged from too low to determine at 3 mg/kg through 0.051 at 100 mg/kg. On day 5, male CD-1 mice had a maximum brain/plasma ratio of 0.256 while female CD-1 mice had a maximum brain/plasma ratio of 0.067. A brain/plasma ratio of less than about 0.5 is considered low penetrance through the blood-brain barrier. Thus TRV 1067 did not appreciably cross the blood-brain barrier in this experiment, in contrast to the prediction of the MDR1-MDCK experiment above.

### Example 7

### In Vivo Brain/Plasma Ratio Determination for TRV 1140

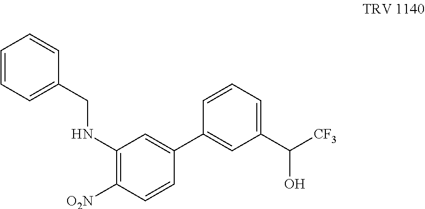 [SMILES: O=[N+]([O-])c1ccc(-c2cccc(C(O)C(F)(F)F)c2)cc1NCc1ccccc1]

TRV 1140 was formulated in 3% N-methyl-2-pyrrolidone, 0.5% Cremaphor, 50% olive oil in water. The compound was formulated at a concentration of 2 mg/mL and dosed in a volume of 10 mL/kg to produce a dose of 20 mg/kg. The formulation was an emulsion. Compound was formulated fresh daily. Mice were dosed once daily for three days. Male ICR mice (S.A. Ace Animals; Boyertown, Pa.) were used for this experiment. Animals were group housed and kept on a standard 12 hr light cycle. Food and water were available ad libitum. On the third day mice were dosed with compound. At the designated time after 20 mg/kg dosing (30 min, 2 hrs, 4 hrs), whole blood was collected via the retro-orbital sinus in heparanized tubes and mixed with deionized water at a 1:1 ratio for hemolyzation. Hemolyzed blood was analyzed for levels of test agent. Blood samples were extracted by a standard protocol known in the art. At designated time points, mice were sacrificed. Brains were removed, weighed, and processed. Brains were homogenized and test agent extracted with an acetonitrile homogenization method (1 mL acetonitrile: 1 g brain). Supernatant samples were stored at −80° C. for subsequent analysis of brain levels of test compound. Brain levels of test agent were analyzed by LC/MS/MS.

Results: 20 mg/kg dosing from 0-4 hr produced total drug exposure in blood of 450 ng×hr/ml and in brain of 694 ng×hr/ml (mean of two samples). Maximum concentration (Cₘₐₓ) was 87.5 ng/ml in blood and 238 ng/ml in brain, at 4 hr. The Brain/plasma ratio was calculated to be 1.54. A brain/plasma ratio of greater than about 0.5 is considered good penetrance through the blood-brain barrier, with a brain/plasma ratio of greater than about 1 considered very good.

TRV 1140 exhibited very good blood and brain exposure levels, with brain penetrance equal to or exceeding blood levels at all time points measured. Thus, TRV 1140 is a highly brain penetrable compound.

### Example 8

### Synthesis of TRV 1190

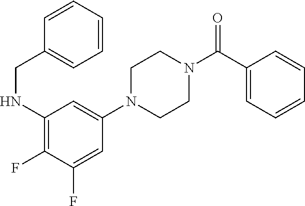 [SMILES: O=C(c1ccccc1)N1CCN(c2cc(F)c(F)c(NCc3ccccc3)c2)CC1]

TRV 1190—(4-(3-(benzylamino)-4,5-difluorophenyl)piperazin-1-yl)(phenyl)methanone Phenyl(4-(3,4,5-trifluorophenyl)piperazin-1-yl)methanone (0.2372 g, 0.74 mmol), benzylamine (10 mL, 91.6 mmol) and NMP (0.74 mL) were sealed in a tube and heated to 170° C. for 3 days. After cooling to room temperature, the mixture was diluted with water and extracted with EtOAc. The combined extracts were washed with 1N HCl(aq), saturated NaHCO₃(aq), H₂O and brine before drying with Na₂SO₄, filtering and concentrating to give the crude oil. This oil was purified via flash chromatography (40% EtOAc/hexane) to afford 0.1319 g (44% yield) of TRV 1190 (4-(3-(benzylamino)-4,5-difluorophenyl)piperazin-1-yl)(phenyl)methanone, as an oil.

¹H NMR (500 MHz, CDCl₃) δ=7.43-7.41 (m, 5H), 7.36-7.35 (m, 4H), 7.31-7.28 (m, 1H), 6.05-6.01 (m, 1H), 5.97 (d, J=6.0 Hz, 1H), 4.40 (s, 1H), 4.34 (d, J=5.5 Hz, 2H), 3.88 (br s, 2H), 3.53 (br s, 2H), 3.08 (br s, 2H), 2.94 (br s, 2H).

### Example 9

### Synthesis of TRV 1186

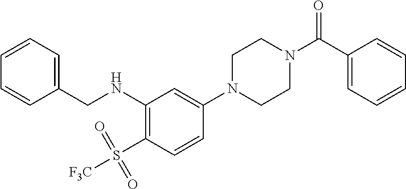 [SMILES: O=C(c1ccccc1)N1CCN(c2ccc(S(=O)(=O)C(F)(F)F)c(NCc3ccccc3)c2)CC1]

### TRV 1186—(4-(3-(benzylamino)-4-((trifluoromethyl)sulfonyl)phenyl)piperazin-1-yl)(phenyl)methanone

Scheme for TRV 1186

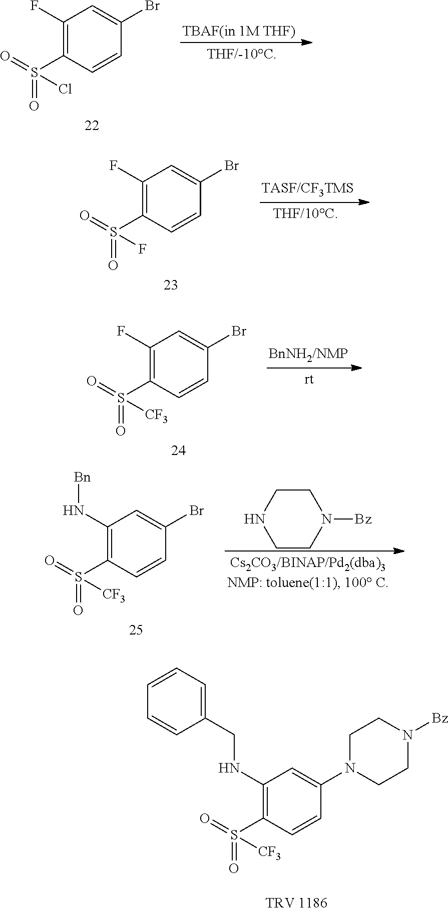

### Synthesis of 4-bromo-2-fluorobenzene-1-sulfonyl fluoride (23)

A 100 mL round bottom flask was charged with 4-bromo-2-fluorobenzene-1-sulfonyl chloride (1.0 g 3.65 mmol) and anhydrous THF (5 mL). The solution was cooled down to −5° C. with an ice bath. Tetrabutylammonium fluoride (1M in THF) (1.15 g, 4.4 mL, 4.38 mmol) was added to the reaction mixture. After the addition was completed, it was stirred for further 30 min at that temperature. It was then quenched with water (20 mL) slowly and extracted with diethyl ether (2×50 mL). Combined organic layer was dried over Na₂SO₄ and concentrated under vacuum to afford the title compound (1.18 g) which was used in next step without further purification.

### Synthesis of 4-bromo-2-fluoro-1-(trifluoromethylsulfonyl)benzene (24)

A 100 mL round bottom flask was charged with 4-bromo-2-fluorobenzene-1-sulfonyl fluoride (1.0 g, 3.89 mmol) and anhydrous THF (30 mL). It was cooled down to 12° C. (((CH₃)₂N)₃S)⁺ (F₂Si (CH₃)₃)⁻, also known as TASF (0.107 g, 0.389 mmol), was added in one portion. Ruppert's reagent (5.53 g, 38.9 mmol, CH₃SiCF₃) was charged slowly to the reaction mixture. The reaction was completed after addition. Water (20 mL) was added to quench the reaction; it was then extracted with diethyl ether (2×50 mL). Combined organic layer was dried over Na2SO4 and concentrated under vacuum to afford the title compound (0.9 g) which was used in next step without further purification. ¹H NMR (500 MHz, CDCl₃): δ 7.85-7.88 (m, 1H), 7.58-7.60 (m, 2H).

### Synthesis of N-benzyl-5-bromo-2-(trifluoromethylsulfonyl) aniline (25)

To a solution of 4-bromo-2-fluoro-1-(trifluoromethylsulfonyl)benzene (0.9 g 2.93 mmol) and diisopropyl ethylamine (0.566 g 4.39 mmol) in NMP (10 mL) was added benzylamine (0.37 g 3.5 mmol). The solution was stirred for 2 h to which was slowly added H₂O (10 mL). The resulting solution was then extracted with EtOAc. The combined organic layer was concentrated under vacuum to give crude product which was purified by column chromatography furnish N-benzyl-5-bromo-2-(trifluoromethylsulfonyl) aniline 25 (0.97 g 84%). ¹H NMR (500 MHz, CDCl₃): δ 4.48 (d, J=5.5 Hz, 2H), 6.41 (m, 1H), 6.94 (d, J=1.5 Hz, 1H), 7.35-7.44 (m, 6H), 7.69 (d, J=8.5 Hz, 1H)

### Synthesis of (4-(3-(benzylamino)-4-(trifluoromethylsulfonyl)phenyl)piperazin-1-yl) (phenyl)methanone (26)

A mixture of N-benzyl-5-bromo-2-(trifluoromethylsulfonyl) aniline (0.5 g, 1.27 mmol), 1-benzoylpiperazine HCl salt (0.430 g, 1.90 mmol), Pd₂(dba)₃ (0.024 g 0.025 mmol), Tol-BINAP (0.034 g, 0.50 mmol) and cesium carbonate (1.24 g, 3.81 mmol) in toluene (15 mL) and DMSO (5 mL) was stirred at 90° C. under N₂ for 12 h. The mixture was filtered through a pad of Celite and washed with EtOAc. The filtrate was then quenched with H₂O and organic layer was evaporated under reduced pressure and the crude product was purified by flash chromatography on silica gel which gave the title compound TRV1186 (4-(3-(benzylamino)-4-((trifluoromethyl) sulfonyl)phenyl)piperazin-1-yl) (phenyl)methanone as a white solid (0.428 g, 67%). ¹H NMR (500 MHz, DMSO-d₆): δ 3.37-3.41 (m, 6H), 3.59 (bs, 2H), 4.50 (d, J=5.5 Hz, 2H), 5.99 (d, J=2.0 Hz, 1H), 6.42-6.44 (dd, J=2.0 Hz, 1H), 6.95 (t, J=5.5, 6.0 Hz, 1H), 7.25 (m, 1H), 7.33-7.43 (m, 5H), 7.44-7.49 (m, 5H).

### Example 10

### Synthesis of TRV 1185

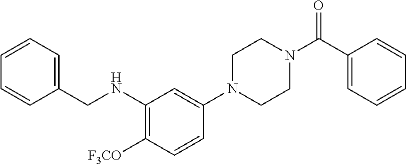 [SMILES: O=C(c1ccccc1)N1CCN(c2ccc(OC(F)(F)F)c(NCc3ccccc3)c2)CC1]

### TRV 1185—(4-(3-(benzylamino)-4-(trifluoromethoxy)phenyl)piperazin-1-yl)(phenyl)methanone

Scheme for TRV 1185

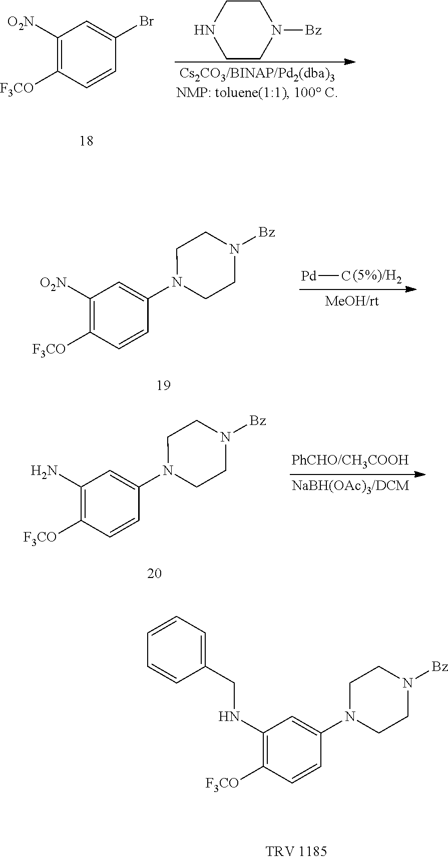

### Synthesis of (4-(3-nitro-4-(trifluoromethoxy)phenyl)piperazin-1-yl)(phenyl)methanone (19)

A mixture of 4-bromo-2-nitro-1-(trifluoromethoxy)benzene 18 (1.5 g, 5.22 mmol), 1-benzoylpiperazine HCl salt (1.77 g, 7.83 mmol), Pd₂(dba)₃ (0.095 g, 0.104 mmol), Tol-BINAP (0.141 g, 0.208 mmol) and cesium carbonate (5.08 g, 15.66 mmol) in toluene (15 mL) and DMSO (10 mL) was stirred at 90° C. under N₂ for 14 h. The mixture was filtered through a pad of Celite and washed with EtOAc. The filtrate was then quenched with H₂O and organic layer was evaporated under reduced pressure and the crude product was purified by flash chromatography on silica gel which gave the title compound 19 as a pale yellow solid (1.4 g, 73% yield). ¹H NMR (500 MHz, CDCl₃): δ 3.00 (bs, 2H), 3.18 (bs, 2H), 3.63 (bs, 2H), 3.98 (bs, 2H), 7.09 (d, J=9.0 Hz, 1H), 7.48 (m, 5H), 7.65 (dd, J=2.5, 8.5, 1 H), 7.98 (d, J=2.0 Hz, 1H)

### Synthesis of (4-(3-amino-4-(trifluoromethoxy)phenyl)piperazin-1-yl)(phenyl)methanone (20)

(4-(3-nitro-4-(trifluoromethoxy)phenyl)piperazin-1-yl)(phenyl)methanone 19 (1.2 g, 3.03 mmol), dissolved in 15 mL of methanol, was hydrogenated for 4 h in the presence of Pd/C 10% (0.150 g). The catalyst was removed by filtration and the solvent was evaporated in vacuo. The residue (0.8 g) was subjected to next step without further purification.

### Synthesis of (4-(3-(benzylamino)-4-(trifluoromethoxy)phenyl)piperazin-1-yl)(phenyl)methanone TRV 1185

To a stirred solution of benzaldehyde (0.127 g, 1.2 mmol) and (4-(3-amino-4-(trifluoromethoxy)phenyl)piperazin-1-yl)(phenyl)methanone 20 (0.365 g, 1.0 mmol) were mixed in DCM (5 mL) at rt under N₂. Sodium triacetoxyborohydride (0.361 g, 1.5 mmol) and glacial AcOH (0.09 g, 1.5 mmol) were added, and the mixture was stirred at rt for 12 h. The reaction mixture was quenched with aqueous saturated NaHCO₃ solution, and the product was extracted with Et₂O. The Et₂O extract was dried (MgSO₄) and concentrated under vacuum. The resultant residue was then purified by column chromatography to afford compound TRV 1185, (4-(3-(benzylamino)-4-(trifluoromethoxy)phenyl)piperazin-1-yl)(phenyl)methanone as white solid (0.36 g, 79%). ¹H NMR (500 MHz, CDCl₃): δ 3.05 (bs, 5H), 3.52 (bs, 3H), 4.40 (d, J=50.5 Hz, 2H), 5.12 (t, J=5.0, 5.5 Hz, 1H), 6.73 (dd, J=1.0, 8.0 Hz, 1H), 6.76 (dd, J=1.0, 1.5 Hz, 1H), 7.05 (d, J=7.5 Hz, 1H), 7.07 (d, J=1.0 Hz, 1H) 7.32 (m, 1H), 7.36-7.45 (m, 8H).

### Example 11

### Synthesis of TRV 1184

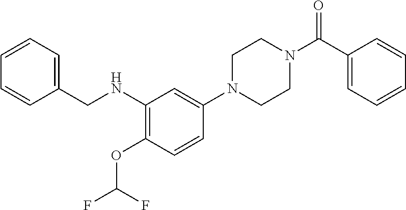 [SMILES: O=C(c1ccccc1)N1CCN(c2ccc(OC(F)F)c(NCc3ccccc3)c2)CC1]

### TRV 1184—(4-(3-(benzyl amino)-4-(difluoromethoxy)phenyl)piperazin-1-yl)(phenyl)methanone

### Scheme for synthesises of TRV 1184

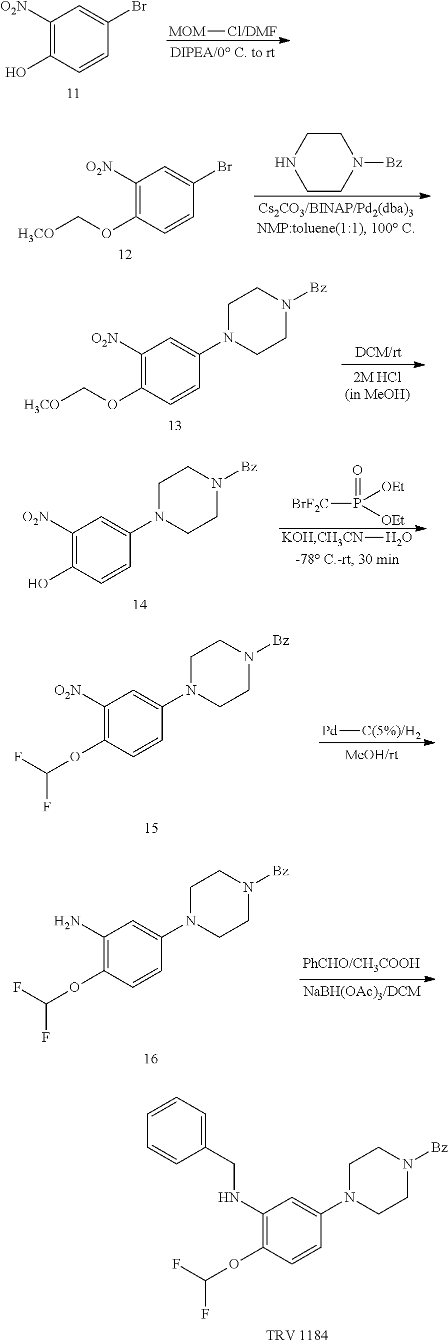

### Synthesis of 4-bromo-1-(methoxymethoxy)-2-nitrobenzene (12)

To a stirred solution of 4-bromo-2-nitrophenol 11 (1.0 g, 4.58 mmol) and diisopropylethylamine (2.3 mL, 13.7 mmol) in anhydrous DMF (15 mL) at 0° C. (ice bath), a solution chloromethyl (methyl)ether (0.44 g, 5.5 mmol) in anhydrous DMF (5 mL) was added dropwise at 0° C., and the mixture was stirred for further 30 min. The reaction was quenched with water (30 mL) and extracted with diethyl ether (3×50 mL). The organic phases were washed with saturated sodium bicarbonate (3×50 mL) and sodium chloride (3×50 mL), and dried with sodium sulfate. The residual oil was purified by column chromatography on silica gel by eluting with a 4:1 mixture of hexane and ethyl acetate to furnish the pure product 12 pale yellow syrup (1.08 g, 90%). ¹H NMR (500 MHz, CDCl₃): δ 3.55 (s, 3H), 5.31 (s, 2H), 7.27 (d, J=9.0 Hz, 1H), 7.65 (dd, J=2.5, 9.0 Hz, 1H), 7.98 (d, J=2.5 Hz, 1H).

### Synthesis of (4-(4-(methoxymethoxy)-3-nitrophenyl)piperazin-1-yl)(Phenyl)methanone (13)

A mixture of 4-bromo-1-(methoxymethoxy)-2-nitrobenzene (1.0 g 3.83 mmol), 1-benzoylpiperazine HCl salt (1.3 g, 5.74 mmol), Pd₂(dba)₃ (0.069 g, 0.076 mmol), Tol-BINAP (0.103 g, 0.153 mmol) and cesium carbonate (3.73 g, 11.49 mmol) in toluene (15 mL) and DMSO (5 mL) was stirred at 90° C. under N₂ for 14 h. The mixture was filtered through a pad of Celite and washed with EtOAc. The filtrate was then quenched with H₂O and organic layer was evaporated under reduced pressure and the crude product was purified by flash chromatography on silica gel which gave the title compound 13 as a pale yellow solid (1.2 g, 84% yield). ¹H NMR (500 MHz, CDCl₃): δ 3.13 (bs, 2H), 3.25 (bs, 2H), 3.55 (s, 3H), 3.65 (bs, 2H), 3.95 (bs, 2H), 5.25 (s, 2H), 7.14 (dd, J=3.0, 9.5 Hz, 1H), 7.29 (d, J=9.5 Hz, 1H), 7.38 (d, J=2.5 Hz, 1H), 7.46 (m, 5H).

### Synthesis of (4-(4-hydroxy-3-nitrophenyl)piperazin-1-yl)(phenyl)methanone (14)

A 2M HCl in methanol solution (11.0 mL) was added to a solution of (4-(4-(methoxymethoxy)-3-nitrophenyl)piperazin-1-yl)(Phenyl)methanone 13 (1.1 g, 2.96 mmol) in dichloromethane (11.0 mL), and the resulting mixture was stirred at rt for 3 h. The reaction was quenched with H₂O and extract twice with dichloromethane, combined organic layer was dried to afford the pure product 14 (0.89 g, 92%). ¹H NMR (500 MHz, CDCl₃): δ 3.14 (bs, 2H), 3.25 (bs, 2H), 3.65 (bs, 2H), 3.95 (bs, 2H), 7.15 (d, J=9.0 Hz, 1H), 7.38 (dd, J=3.0, 9.0 Hz, 1H), 7.47 (m, 5H) 7.55 (d, J=3.0 Hz, 1H), 10.3 (s, 1H).

### Synthesis of (4-(4-(difluoromethoxy)-3-nitrophenyl)piperazin-1-yl)(phenyl)methanone (15)

Diethyl bromodifluoromethylphosphonate (0.816 g, 3.05 mmol) was added in one portion to a cooled (−78° C.) solution of the (4-(4-hydroxy-3-nitrophenyl)piperazin-1-yl)(phenyl)methanone 14 (1 mmol) and KOH (20 mmol) in CH₃CN—H₂O (10 mL, 1:1), placed in a round bottom flask with a magnetic stirrer. The reaction mixture was allowed to warm to rt. After 30 min, the reaction mixture was diluted with ether (10 mL) and the organic phase was separated. The water phase was then washed with a further amount of ether (10 mL), and the combined organic fractions were dried over anhydrous Na₂SO₄. Evaporation of the solvent gave a crude product that was purified by column chromatography to afford title compound 15 (0.51 g, 84%). ¹H NMR (500 MHz, CDCl₃): δ 3.28 (bs, 2H), 3.34 (bs, 2H), 3.66 (bs, 2H), 3.98 (bs, 2H), 6.72 (t, J=73.5 Hz, 1H), 7.14 (dd, J=3.0, 4.5 Hz, 1H), 7.38 (dd, J=3.0, 9.0 Hz, 1H), 7.55 (d, J=4.5 Hz, 1H), 7.49 (m, 5H).

### Synthesis of (4-(3-amino-4-(difluoromethoxy)phenyl)piperazin-1-yl)(phenyl)methanone (16)

(4-(4-(difluoromethoxy)-3-nitrophenyl)piperazin-1-yl)(phenyl)methanone 15 (0.35 g, 1 mmol), dissolved in 10 mL of methanol, was hydrogenated for 4 h in the presence of Pd/C 10% (0.07 g). The catalyst was removed by filtration and the solvent was evaporated in vacuo. The residue (0.28 g) was subjected to next step without further purification.

### Synthesis of (4-(3-(benzyl amino)-4-(difluoromethoxy)phenyl)piperazin-1-yl)(phenyl)methanone (TRV 1184)

To a stirred solution of benzaldehyde (0.036 mL, 0.345 mmol) and (4-(3-amino-4-(difluoromethoxy)phenyl)piperazin-1-yl)(phenyl)methanone 16 (0.1 g, 0.228 mmol) were mixed in DCM (5 mL) at rt under N₂. Sodium triacetoxyborohydride (0.091 g, 0.432 mmol) and glacial AcOH (0.026 g, 0.432 mmol) were added, and the mixture was stirred at rt for 12 h. The reaction mixture was quenched with aqueous saturated NaHCO₃ solution, and the product was extracted with Et₂O. The Et₂O extract was dried (MgSO₄) and concentrated under vacuum. The resultant residue was then purified by column chromatography to afford compound Synthesis of (4-(3-(benzyl amino)-4-(difluoromethoxy)phenyl)piperazin-1-yl)(phenyl)methanone, TRV 1184 as a white solid (0.11 g, 88%). ¹H NMR (500 MHz, DMSO): δ 2.94 (bs, 2H), 3.02 (bs, 2H), 3.42 (bs, 2H), 3.68 (bs, 2H), 4.35 (d, J=6.5 Hz, 2H), 5.86 (t, J=6.0, 6.5 Hz, 1H), 6.10-6.12 (dd, J=2.5 Hz, 1H), 6.14 (d, J=2.5 Hz, 1H), 6.78-7.08 (t, J=75 Hz, 1H), 6.88 (d, J=8.5 Hz, 1H), 7.23 (t, J=7.0, 7.5 Hz, 1H), 7.29-7.44 (m, 10H).

### Example 12

### Synthesis of TRV 1179

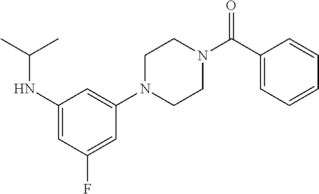 [SMILES: CC(C)Nc1cc(F)cc(N2CCN(C(=O)c3ccccc3)CC2)c1]

### TRV 1179—(4-(3-fluoro-5-(isopropylamino)phenyl)piperazin-1-yl)(phenyl)methanone

Scheme for synthesis of TRV 1179

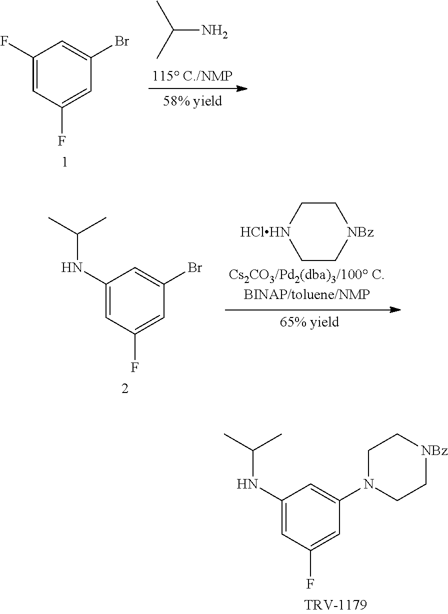

3,5-difluoro-1-bromobenzene (2.00 g, 10.36 mmol), isopropyl amine (3.6 mL, 41.4 mmol) and NMP (10 mL) were added to a tube. The tube was sealed and heated overnight at 115° C. at which point NMR analysis suggested only 40% conversion. Two additional equivalents of isopropylamine were added and the reaction was stirred for another 24 hours at 115° C. After cooling, the reaction mixture was diluted with water and EtOAc. The aqueous layer was back-extracted with EtOAc (3×). The combined organic layers were then washed with H₂O, 1N HCl (2×), saturated NaHCO₃, H₂O and brine before drying with Na₂SO₄. The material was filtered and concentrated under reduced pressure to give 1.4 g of the crude oil 7. Purification of this material was not required. This aniline (0.5298 g, 2.3 mmol), benzoylpiperazine hydrochloride (0.6215 g, 2.7 mmol) and Cs₂CO₃ (2.25 g, 6.9 mmol) were added to a tube. The tube was evacuated and flushed with argon for three cycles. Toluene (7.0 mL) and NMP (4.2 mL) were then added and the mixture was degassed with argon for 30 minutes. Pd₂(dba)₃ (0.0421 g, 0.046 mmol) and BINAP (0.0573 g, 0.092 mmol) were then added, the tube was sealed and heated overnight at 100° C. After cooling, the mixture was diluted with water and EtOAc. The aqueous layer was back-extracted with EtOAc (3×). The combined organic layers were then washed with H₂O, 1N HCl (2×), saturated NaHCO₃, H₂O and brine before drying with Na₂SO₄. The material was filtered and concentrated under reduced pressure to give a crude oil. Purification of this material via flash chromatography (30% EtOAc/hexane) gave 0.5106 g (65% yield) of TRV 1179, (4-(3-fluoro-5-(isopropylamino)phenyl)piperazin-1-yl)(phenyl)methanone

¹H NMR (500 MHz, CDCl₃) δ=7.43 (s, 5H), 5.97-5.94 (m, 1H), 5.88-5.85 (m, 2H), 3.90 (br s, 2H), 3.56 (br s, 4H), 3.22 (br s, 2H), 3.07 (br s, 2H), 1.20 (d, J=5.5 Hz, 6H).

### Example 13

### Synthesis of TRV 1178

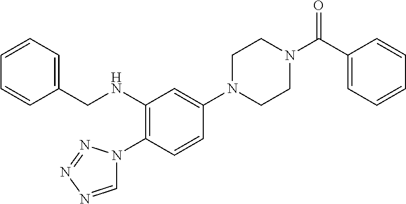 [SMILES: O=C(c1ccccc1)N1CCN(c2ccc(-n3cnnn3)c(NCc3ccccc3)c2)CC1]

### TRV 1178—(4-(3-(benzylamino)-4-(1H-tetrazol-1-yl)phenyl)piperazin-1-yl)(phenyl)methanone

To a solution of 4-bromo-2-fluoro-1-nitrobenzene (0.5 g, 2.29 mmol) and diisopropyl ethylamine (1.38 mL, 3.44 mmol) in NMP (5 mL) was added benzylamine (0.3 mL, 2.7 mmol). The solution was stirred for 14 h to which was slowly added H₂O (5 mL). The resulting yellow precipitate was filtered, washed with 2 mL of water, dried (high vacuum, 14 h) to furnish N-benzyl-5-bromo-2-nitrobenzenamine (670 mg, 2.18 mmol), 95% yield. ¹H NMR (500 MHz, CDCl₃): δ 4.55 (d, J=5.5 Hz, 2H), 6.83 (dd, J=2 Hz, 1H), 7.06 (d, J=1.9 Hz, 1H), 7.38 (m, 5H), 8.10 (d, J=9.1 Hz, 1H), 8.42 (bs, 1H)

### Synthesis of N,N-dibenzyl-5-bromo-2-nitroaniline (4)

To a stirred solution of N-benzyl-5-bromo-2-nitroaniline (2.0 g, 6.5 mmol) and benzyl bromide (0.95 mL, 7.8 mmol) in Toluene (20 mL) was added KOH (0.728 g, 13.0 mmol) at rt. To this mixture tBu₄NI (cat. ˜50 mg) was added and reaction was stirred 4 hrs. It was then quenched with H₂O and extracted with ethyl acetate. Combined organic layer was then evaporated under vacuum and finally residue was purified by column chromatography to afford the title compound (1.25 g, 50%). ¹H NMR (500 MHz, CDCl₃): δ 4.32 (s, 4H), 6.80 (dd, J=2 Hz, 1H), 7.05 (d, J=2.0 Hz, 1H), 7.34 (m, 5H), 8.03 (d, J=9.0 Hz, 1H),

### Synthesis of N,N-dibenzyl-2-nitro-5-(piperazin-1-yl) aniline (5)

A mixture of N,N-dibenzyl-5-bromo-2-nitroaniline (1.0 g, 2.52 mmol), piperazine (0.325 g, 1.5 mmol), K₃PO₄ (1.06 g, 5.04 mmol), CuI (0.05 g, 0.25 mmol), and proline (0.06 g 0.504 mmol) in 15 mL of DMSO was heated at 90° C. The cooled mixture was partitioned between water and ethyl acetate. The organic layer was separated, and the aqueous layer was extracted with ethyl acetate. The combined organic layers were washed with brine, dried over Na₂SO₄, and concentrated in vacuum. The residual oil (2.5 g) was used in next step without further purification.

### Synthesis of (4-(3-(dibenzylamino)-4-nitrophenyl)piperazin-1-yl)(phenyl)methanone (6)

A solution of N,N-dibenzyl-2-nitro-5-(piperazin-1-yl) aniline 5 (2.5 g, 6.21 mmol) and triethylamine (1.7 mL, 12.4 mmol) was stirred in CH₂Cl₂ (20 mL) at room temperature for 10 min. A solution of benzoyl chloride (0.86 mL, 7.46 mmol) was added slowly into the above solution maintained between 0-5° C. for 15 min. The mixture was stirred at room temperature for 1 h. The reaction mixture was extracted with CH₂Cl₂. The combined organic layers were dried by anhydrous sodium sulphate. The solvent was removed under vacuum to give title compound 6 which was purified by column chromatography (2.5 g, 80%). ¹H NMR (500 MHz, CDCl₃): δ 3.18 (bs, 2H), 3.31 (bs, 2H), 3.52 (bs, 2H), 3.84 (bs, 2H), 4.23 (s, 4H), 6.21 (d, J=1.5 Hz, 1H), 6.39 (dd, J=1.5 Hz, 1H), 7.22-7.46 (m, 5H), 7.97 (d, J=7.0 Hz, 1H).

### Synthesis of (4-(4-amino-3-(dibenzylamino)phenyl)piperazin-1-yl)(phenyl)methanone (7)

To a stirred solution of (4-(3-(dibenzylamino)-4-nitrophenyl)piperazin-1-yl)(phenyl)methanone (2.0 g, 3.95 mmol) in EtOAc (20 mL) was added SnCl₂.H₂O (2.24 g, 1.85 mmol) at room temperature. The resulting solution was stirred overnight then quenched with water; organic layer was then separated, dried over Na₂SO₄ and concentrated under vacuum. The residue was purified by column Chromatography to afford title compound 7 (1.2 g, 65%). ¹H NMR (500 MHz, CDCl₃): δ 2.83 (bs, 2H), 2.99 (bs, 2H), 3.55 (bs, 2H), 3.92 (bs, 2H), 3.96 (bs, 2H), 4.05 (s, 4H), 6.49 (s, 1H), 6.60 (d, J=5.0 Hz, 1H), 6.71 (d, J=6.0 Hz, 1H), 7.22-7.31 (m, 10H), 7.45 (m, 5H).

### Synthesis of (4-(3-(dibenzylamino)-4-(1H-tetrazol-1-yl)phenyl)piperazin-1-yl)(phenyl)methanone (8)

A 250 mL round-bottom flask was charged with a solution of (4-(4-amino-3-(dibenzylamino) phenyl)piperazin-1-yl)(phenyl)methanone 7 (1 g, 2.1 mmole) in AcOH (10 mL). To this was added NaN₃ (0.204 g, 3.15 mmole). To the mixture was added trimethoxymethane (0.34 mL, 3.15 mmole) and the resulting solution was stirred for 4 hr at 90° C. The resulting mixture was concentrated under vacuum and diluted with 15 mL water and extracted with ethyl acetate (2×50 mL). Final purification was done by column chromatography to afford the title compound 8 (1.0 g, 95%). ¹H NMR (500 MHz, CDCl₃): δ 3.19 (bs, 2H), 3.25 (bs, 2H), 3.64 (bs, 2H), 3.96 (bs, 2H), 3.88 (s, 4H), 6.67 (d, J=2.5 Hz, 1H), 6.76 (dd, J=2.5 Hz, 1H), 7.05 (m, 4H), 7.26-7.33 (m, 8H), 7.38 (dJ=8.5 Hz, 1H), 7.50 (m, 5H), 8.81 (s, 1H).

### Synthesis of (4-(3-amino-4-(1H-tetrazol-1-yl)phenyl)piperazin-1-yl)(phenyl)methanone (9)

(4-(3-(dibenzylamino)-4-(1H-tetrazol-1-yl)phenyl)piperazin-1-yl)(phenyl)methanone 8 (0.520 g, 1.0 mmol) was dissolved in 2M HCl in methanol (12 mL) under argon. To this was added 5% Pd—C (0.1 g), the resultant mixture was then stirred under H₂ atmosphere overnight. After completion of reaction by TLC and usual work up. Finally the residue was purified by column chromatography to afford title compound 9 as white solid (0.3 g, 85%). ¹H NMR (500 MHz, CDCl₃): δ 3.27-3.39 (m, 5H), 3.73 (m, 3H), 4.03 (bs, 2H), 6.52 (d, J=2.0 Hz, 1H), 6.57 (m, 1H), 7.16 (d, J=8.0 Hz, 1H), 7.50 (m, 5H), 8.84 (s, 1H).

### Synthesis of (4-(3-(benzyl amino)-4-(1H-tetrazol-1-yl)phenyl)piperazin-1-yl)(phenyl)methanone TRV 1178

To a stirred solution of benzaldehyde (0.12 mL, 0.127 g, 1.2 mmol) and (4-(3-amino-4-(1H-tetrazol-1-yl)phenyl)piperazin-1-yl)(phenyl)methanone 9 (0.350 g, 1.0 mmol) were mixed in DCM (5 mL) at rt under N₂. Sodium triacetoxyborohydride (0.316 g, 1.5 mmol) and glacial AcOH (0.090 g, 1.5 mmol) were added, and the mixture was stirred at rt for 12 h. The reaction mixture was quenched with aqueous saturated NaHCO, solution, and the product was extracted with ethyl acetate. The ethyl acetate extract was dried over (MgSO₄) and concentrated under vacuum. The resultant residue was then purified by column chromatography to afford compound (4-(3-(benzyl amino)-4-(1H-tetrazol-1-yl)phenyl)piperazin-1-yl)(phenyl)methanone TRV 1178 as a white solid (0.290 g 65%). ¹H NMR (500 MHz, CDCl₃): δ 3.08 (bs, 2H), 3.18 (bs, 2H), 3.39 (bs, 2H), 3.68 (bs, 2H), 4.30 (d, J=6.0 Hz, 2H), 5.96 (t, J=5.5, 6.0 Hz, 1H), 6.17 (d, J=2.0 Hz, 1H), 6.29 (dd, J=2.0, 2.5 Hz, 1H), 7.05 (d, J=9.0 Hz, 1H), 7.20-7.48 (m, 10H), 9.66 (s, 1H).

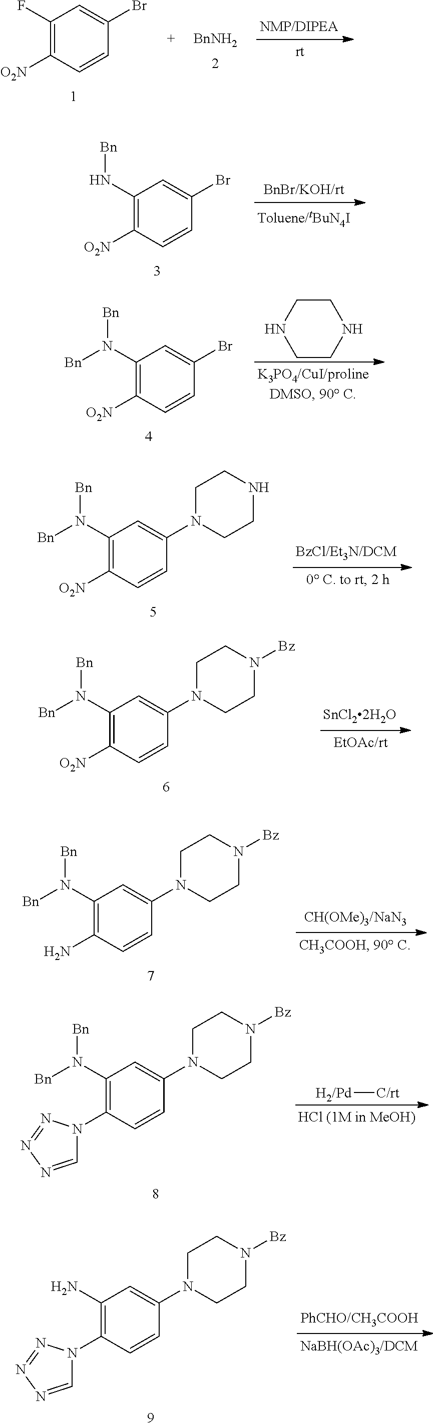

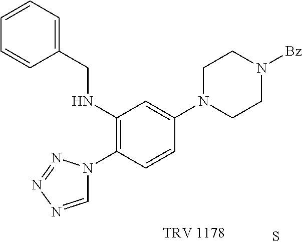

Scheme for TRV 1178

### Example 14

### Synthesis of TRV 1176

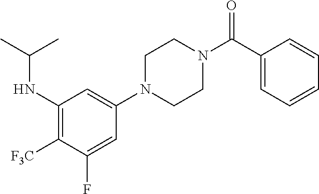 [SMILES: CC(C)Nc1cc(N2CCN(C(=O)c3ccccc3)CC2)cc(F)c1C(F)(F)F]

### TRV 1176—(4-(3-fluoro-5-(isopropylamino)-4-(trifluoromethyl)phenyl)piperazin-1-yl)(phenyl)methanone

Scheme for TRV 1176

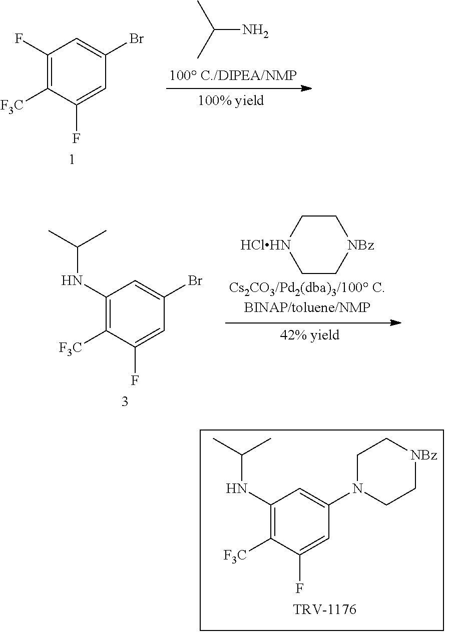

3,5-Difluoro-4-(trifluoromethyl)bromobenzene (0.9376 g, 3.59 mmol), isopropyl amine (0.34 mL, 3.95 mmol), DIPEA (0.94 mL, 5.4 mmol) and NMP (4.8 mL) were added to a tube. The tube was sealed and heated overnight at 115° C. After cooling, the reaction mixture was diluted with water and EtOAc. The aqueous layer was back-extracted with EtOAc (3×). The combined organic layers were then washed with H₂O, 1N HCl (2×), saturated NaHCO₃, H₂O and brine before drying with Na₂SO₄. The material was filtered and concentrated under reduced pressure to give the crude oil of 3. Purification of this material was not required. This aniline (0.6248 g, 2.80 mmol), benzoylpiperazine hydrochloride (0.5668 g, 2.5 mmol) and Cs₂CO₃ (2.02 g, 6.2 mmol) were added to a tube. The tube was evacuated and flushed with argon for three cycles. Toluene (6.3 mL) and NMP (3.8 mL) were then added and the mixture was degassed with argon for 30 minutes. Pd₂(dba)₃ (0.0385 g, 0.042 mmol) and BINAP (0.0523 g, 0.084 mmol) were then added, the tube was sealed and heated overnight at 100° C. After cooling, the mixture was diluted with water and EtOAc. The aqueous layer was back-extracted with EtOAc (3×). The combined organic layers were then washed with H₂O, 1N HCl (2×), saturated NaHCO₃, H₂O and brine before drying with Na₂SO₄. The material was filtered and concentrated under reduced pressure to give a crude oil. Purification of this material via flash chromatography (30% EtOAc/hexane) have 0.354 g (42% yield) of TRV 1176, (4-(3-fluoro-5-(isopropylamino)-4-(trifluoromethyl)phenyl)piperazin-1-yl)(phenyl)methanone

¹H NMR (500 MHz, CDCl₃) δ=7.46-7.42 (m, 5H), 5.93 (dd, J=15, 1.5 Hz, 1H), 5.85 (s, 1H), 4.47 (d, J=3 Hz, 1H), 3.91 (br s, 2H), 3.64-3.59 (m, 1H), 3.58 (br s, 2H), 3.32 (br s, 2H), 3.18 (br s, 2H), 1.23 (d, J=6.5 Hz, 6H).

### Example 15

### Synthesis of TRV 1175

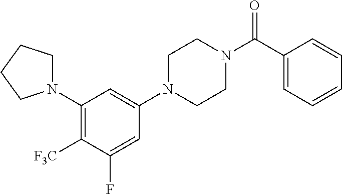 [SMILES: O=C(c1ccccc1)N1CCN(c2cc(F)c(C(F)(F)F)c(N3CCCC3)c2)CC1]

### TRV 1175—(4-(3-fluoro-5-(pyrrolidin-1-yl)-4-(trifluoromethyl)phenyl)piperazin-1-yl)(phenyl)methanone

Scheme for TRV 1175

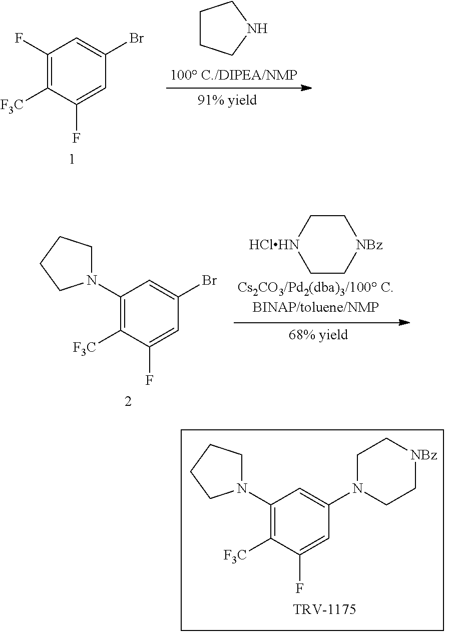

3,5-Difluoro-4-(trifluoromethyl)bromobenzene (0.9203 g, 3.53 mmol), pyrrolidine (0.32 mL, 3.88 mmol), DIPEA (0.92 mL, 3.88 mmol) and NMP (4.7 mL) were added to a tube. The tube was sealed and heated overnight at 100° C. After cooling, the reaction mixture was diluted with water and EtOAc. The aqueous layer was back-extracted with EtOAc (3×). The combined organic layers were then washed with H₂O, 1N HCl (2×), saturated NaHCO₃, H₂O and brine before drying with Na₂SO₄. The material was filtered and concentrated under reduced pressure to give a crude oil. This oil was purified via flash column chromatography (3% EtOAc/hexane) to afford 1.00 g (91% yield) of the aniline 2. This aniline (0.6261 g, 2.0 mmol), benzoylpiperazine hydrochloride (0.544 g, 2.4 mmol) and Cs₂CO₃ (1.95 g, 6.0 mmol) were added to a tube. The tube was evacuated and flushed with argon for three cycles. Toluene (6.0 mL) and NMP (3.6 mL) were then added and the mixture was degassed with argon for 30 minutes. Pd₂(dba)₃ (0.0366 g, 0.04 mmol) and BINAP (0.0498 g, 0.08 mmol) were then added, the tube was sealed and heated overnight at 100° C. After cooling, the mixture was diluted with water and EtOAc. The aqueous layer was back-extracted with EtOAc (3×). The combined organic layers were then washed with H₂O, 1N HCl (2×), saturated NaHCO₃, H₂O and brine before drying with Na₂SO₄. The material was filtered and concentrated under reduced pressure to give a crude oil. Purification of this material via flash chromatography (30% EtOAc/hexane) gave 0.5729 g (68% yield) of TRV 1175, (4-(3-fluoro-5-(pyrrolidin-1-yl)-4-(trifluoromethyl)phenyl)piperazin-1-yl)(phenyl)methanone ¹H NMR (500 MHz, CDCl₃) δ=7.47-7.42 (m, 5H), 6.10 (d, J=13 Hz, 1H), 6.09 (s, 1H), 3.91 (br s, 2H), 3.58 (br s, 2H), 3.34 (br s, 2H), 3.29-3.25 (m, 4H), 3.19 (br s, 2H), 1.94-1.90 (m, 4H).

### Example 16

### Synthesis of TRV 1170

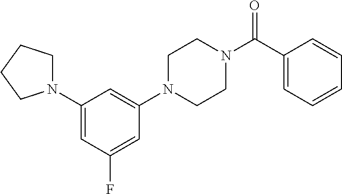 [SMILES: O=C(c1ccccc1)N1CCN(c2cc(F)cc(N3CCCC3)c2)CC1]

### TRV 1170 (4-(3-fluoro-5-(pyrrolidin-1-yl)phenyl)piperazin-1-yl)(phenyl)methanone

Scheme for TRV 1170

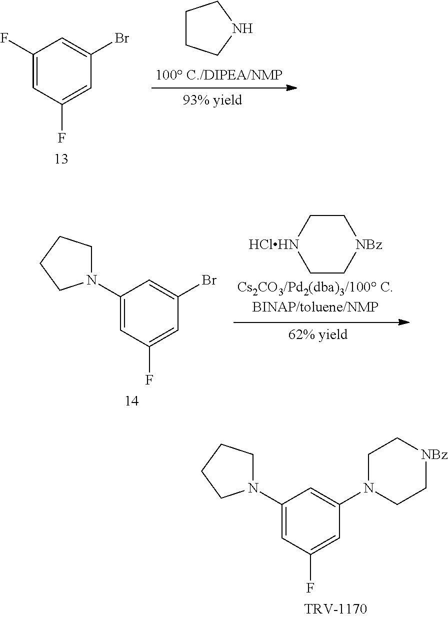

1-bromo-3,5-difluorobenzene (1.00 g, 5.18 mmol), pyrrolidine (0.47 mL, 5.7 mmol), DIPEA (1.4 mL, 7.77 mmol) and NMP (6.9 mL) were added to a tube. The tube was sealed and stirred at 100° C. overnight. The reaction mixture was diluted with water and EtOAc. The aqueous layer was back-extracted with EtOAc (3×). The combined organic layers were then washed with H₂O, 1N HCl (2×), saturated NaHCO₃, H₂O and brine before drying with Na₂SO₄. The material was filtered and concentrated under reduced pressure to give 1.178 g of a crude yellow solid. Purification of this material was not required. This aniline (0.4902 g, 2.0 mmol), benzoylpiperazine hydrochloride (0.5441 g, 2.4 mmol) and Cs₂CO₃ (1.95 g, 6.0 mmol) were added to a tube. The tube was evacuated and flushed with argon for three cycles. Toluene (6 mL) and NMP (3.6 mL) were then added and the mixture was degassed with argon for 30 minutes. Pd₂(dba)₃ (0.0366 g, 0.04 mmol) and BINAP (0.0498 g, 0.08 mmol) were then added, the tube was sealed and heated overnight at 100° C. After cooling, the mixture was diluted with water and EtOAc. The aqueous layer was back-extracted with EtOAc (3×). The combined organic layers were then washed with H₂O, 1N HCl (2×), saturated NaHCO₃, H₂O and brine before drying with Na₂SO₄. The material was filtered and concentrated under reduced pressure to give a crude oil. Purification of this material via recrystallization via flash chromatography (30% EtOAc/hexane) gave 0.4392 g (62% yield) of TRV 1170, (4-(3-fluoro-5-(pyrrolidin-1-yl)phenyl)piperazin-1-yl)(phenyl)methanone. ¹H NMR (500 MHz, CDCl₃) δ=7.43 (s, 5H), 5.96 (d, J=11.5 Hz, 1H), 5.85 (d, J=12.0 Hz, 1H), 5.81 (s, 1H), 3.92 (br s, 2H), 3.83 (br s, 2H), 3.26-3.23 (m, 6H), 3.10 (br s, 2H), 2.00-1.98 (m, 4H).

### Example 17

### Synthesis of TRV 1169

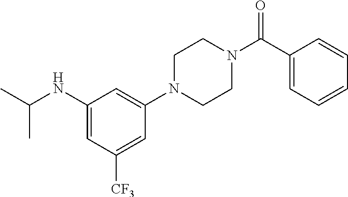 [SMILES: CC(C)Nc1cc(N2CCN(C(=O)c3ccccc3)CC2)cc(C(F)(F)F)c1]

### TRV 1169—(4-(3-(isopropylamino)-5-(trifluoromethyl)phenyl)piperazin-1-yl)(phenyl)methanone

Scheme for TRV 1169

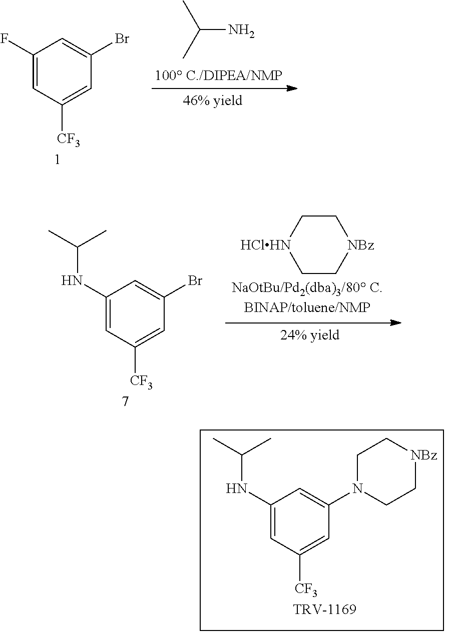

3-bromo-5-fluorobenzotrifluoride (0.739 g, 3.0 mmol), isopropyl amine (0.40 mL, 4.56 mmol), DIPEA (0.79 mL, 4.56 mmol) and NMP (4 mL) were added to a tube. The tube was sealed and heated overnight at 115° C. After cooling, the reaction mixture was diluted with water and EtOAc. The aqueous layer was back-extracted with EtOAc (3×). The combined organic layers were then washed with H₂O, 1N HCl (2×), saturated NaHCO₃, H₂O and brine before drying with Na₂SO₄. The material was filtered and concentrated under reduced pressure to give 0.3379 g of a crude oil. Purification of this material was not required. This aniline (0.3379 g, 1.20 mmol), benzoylpiperazine hydrochloride (0.3264 g, 1.44 mmol) and Cs₂CO₃ (1.17 g, 3.6 mmol) were added to a tube. The tube was evacuated and flushed with argon for three cycles. Toluene (3.6 mL) and NMP (2.2 mL) were then added and the mixture was degassed with argon for 30 minutes. Pd₂(dba)₃ (0.0219 g, 0.024 mmol) and BINAP (0.0299 g, 0.048 mmol) were then added, the tube was sealed and heated overnight at 100° C. After cooling, the mixture was diluted with water and EtOAc. The aqueous layer was back-extracted with EtOAc (3×). The combined organic layers were then washed with H₂O, 1N HCl (2×), saturated NaHCO₃, H₂O and brine before drying with Na₂SO₄. The material was filtered and concentrated under reduced pressure to give a crude oil. Purification of this material via flash chromatography (30% EtOAc/hexane) have 0.1142 g (24% yield) of TRV 1169, (4-(3-(isopropylamino)-5-(trifluoromethyl)phenyl)piperazin-1-yl)(phenyl)methanone.

¹H NMR (700 MHz, CDCl₃) δ=7.46-7.41 (m, 5H), 6.45 (s, 1H), 6.35 (s, 1H), 6.21 (s, 1H), 3.93 (br s, 2H), 3.65-3.60 (m, 2H), 3.58 (br s, 2H), 3.26 (br s, 2H), 3.12 (br s, 2H), 1.70 (d, 6.3 Hz, 6H).

### Example 18

### Synthesis of 1168

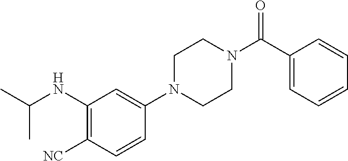 [SMILES: CC(C)Nc1cc(N2CCN(C(=O)c3ccccc3)CC2)ccc1C#N]

### TRV 1168—4-(4-benzoylpiperazin-1-yl)-2-(isopropylamino)benzonitrile

Scheme for TRV 1168

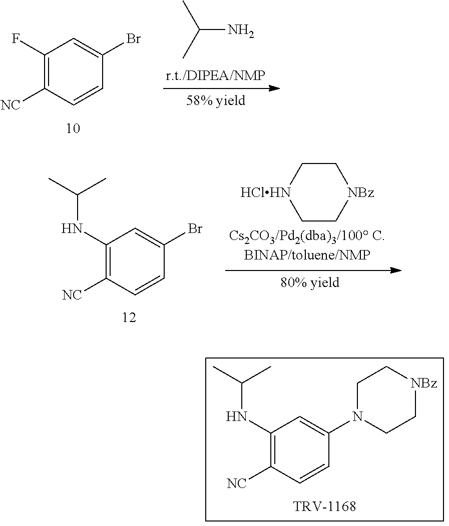

4-bromo-2-fluorobenzonitrile (5.00 g, 25 mmol), isopropyl amine (2.6 mL, 30 mmol), DIPEA (6.5 mL, 37.5 mmol) and NMP (20 mL) were added to a tube. The tube was sealed and stirred at room temperature overnight. The reaction mixture was diluted with water and EtOAc. The aqueous layer was back-extracted with EtOAc (3×). The combined organic layers were then washed with H₂O, 1N HCl (2×), saturated NaHCO₃, H₂O and brine before drying with Na₂SO₄. The material was filtered and concentrated under reduced pressure to give a crude yellow solid. This material was recrystallized from EtOAc (solvent) and hexane (anti-solvent) to afford 3.44 g (58% yield) of yellow needles. This aniline (1.00 g, 4.18 mmol), benzoylpiperazine hydrochloride (1.14 g, 5.02 mmol) and Cs₂CO₃ (4.09 g, 12.54 mmol) were added to a tube. The tube was evacuated and flushed with argon for three cycles. Toluene (12.9 mL) and NMP (7.8 mL) were then added and the mixture was degassed with argon for 30 minutes. Pd₂(dba)₃ (0.0765 g, 0.0836 mmol) and BINAP (0.1040 g, 0.167 mmol) were then added, the tube was sealed and heated overnight at 100° C. After cooling, the mixture was diluted with water and EtOAc. The aqueous layer was back-extracted with EtOAc (3×). The combined organic layers were then washed with H₂O, 1N HCl (2×), saturated NaHCO₃, H₂O and brine before drying with Na₂SO₄. The material was filtered and concentrated under reduced pressure to give a crude solid. Purification of this material via recrystallization from EtOAc (solvent) and hexane (anti-solvent) gave 1.1614 g (80% yield) of TRV 1168, 4-(4-benzoylpiperazin-1-yl)-2-(isopropylamino)benzonitrile. ¹H NMR (700 MHz, CDCl₃) δ=7.46-7.41 (m, 5H), 7.24 (d, J=8.4 Hz, 1H), 6.18 (dd, J=8.4, 2.1 Hz, 1H), 5.99 (d, J=2.1 Hz, 1H), 4.30 (d, J=7.7 Hz, 1H), 3.92 (br s, 2H), 3.70-3.65 (m, 1H), 3.59 (m, 2H), 3.38 (br s, 2H), 3.23 (br s, 2H), 1.25 (d, J=6.3 Hz, 6H).

### Example 19

### Synthesis of TRV 1167

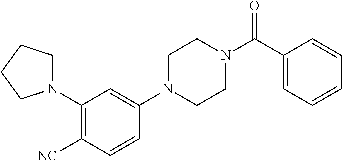 [SMILES: N#Cc1ccc(N2CCN(C(=O)c3ccccc3)CC2)cc1N1CCCC1]

### TRV 1167 4-(4-benzoylpiperazin-1-yl)-2-(pyrrolidin-1-yl)benzonitrile

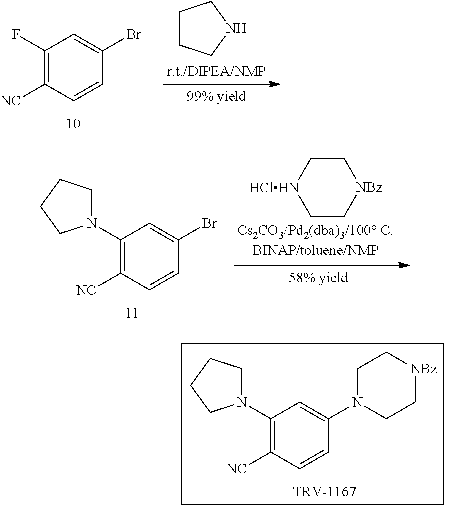

4-bromo-2-fluorobenzonitrile (5.00 g, 25 mmol), pyrrolidine (2.5 mL, 30 mmol), DIPEA (6.5 mL, 37.5 mmol) and NMP (20 mL) were added to a tube. The tube was sealed and stirred at room temperature overnight. The reaction mixture was diluted with water and EtOAc. The aqueous layer was back-extracted with EtOAc (3×). The combined organic layers were then washed with H₂O, 1N HCl (2×), saturated NaHCO₃, H₂O and brine before drying with Na₂SO₄. The material was filtered and concentrated under reduced pressure to give 6.2 g of a crude yellow solid. Purification of this material was not required. This aniline (1.00 g, 3.98 mmol), benzoylpiperazine hydrochloride (1.08 g, 4.78 mmol) and Cs₂CO₃ (3.98 g, 11.94 mmol) were added to a tube. The tube was evacuated and flushed with argon for three cycles. Toluene (12.3 mL) and NMP (7.4 mL) were then added and the mixture was degassed with argon for 30 minutes. Pd₂(dba)₃ (0.0729 g, 0.0796 mmol) and BINAP (0.0990 g, 0.159 mmol) were then added, the tube was sealed and heated overnight at 100° C. After cooling, the mixture was diluted with water and EtOAc. The aqueous layer was back-extracted with EtOAc (3×). The combined organic layers were then washed with H₂O, 1N HCl (2×), saturated NaHCO₃, H₂O and brine before drying with Na₂SO₄. The material was filtered and concentrated under reduced pressure to give a crude solid. Purification of this material via recrystallization from EtOAc (solvent) and hexane (anti-solvent) gave 0.8301 g (58% yield) of TRV 1167, 4-(4-benzoylpiperazin-1-yl)-2-(pyrrolidin-1-yl)benzonitrile.

¹H NMR (700 MHz, CDCl₃)=7.46-7.41 (m, 5H), 7.31 (d, J=9.1 Hz, 1H), 6.23 (dd, J=9.1, 2.1 Hz, 1H), 5.93 (d, J=2.1 Hz, 1H), 3.91 (br s, 2H), 3.58-3.57 (m, 6H), 3.36 (br s, 2H), 3.32 (br s, 2H), 2.00-1.96 (m, 4H).

### Example 20

### Synthesis of TRV 1166

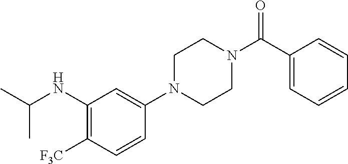 [SMILES: CC(C)Nc1cc(N2CCN(C(=O)c3ccccc3)CC2)ccc1C(F)(F)F]

### TRV 1166 (4-(3-(isopropylamino)-4-(trifluoromethyl)phenyl)piperazin-1-yl)(phenyl)methanone

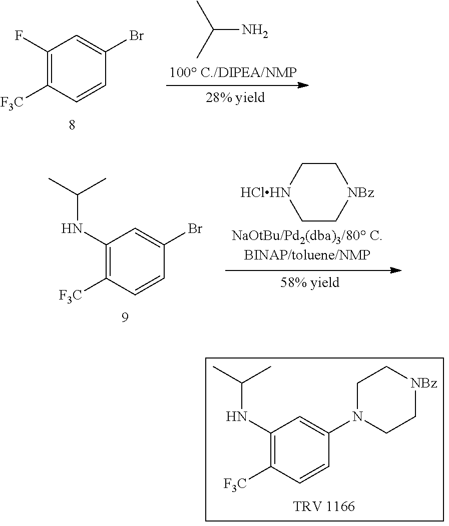

Scheme for TRV 1166

4-bromo-2-fluorobenzotrifluoride (0.5181 g, 2.13 mmol), isopropyl amine (0.22 mL, 2.56 mmol), DIPEA (0.56 mL, 3.2 mmol) and NMP (3 mL) were added to a tube. The tube was sealed and heated overnight at 100° C. After cooling, the reaction mixture was diluted with water and EtOAc. The aqueous layer was back-extracted with EtOAc (3×). The combined organic layers were then washed with H₂O, 1N HCl (2×), saturated NaHCO₃, H₂O and brine before drying with Na₂SO₄. The material was filtered and concentrated under reduced pressure to give 0.1521 g of crude oil which was a 4:6 mixture of starting material to product, respectively. This material was dissolved in NMP (3 mL), treated with DIPEA (0.07 mL, 0.38 mmol) and isopropyl amine (0.2 mL) and heated in a sealed tube at 115° C. overnight. After cooling, the reaction mixture was diluted with water and EtOAc. The aqueous layer was back-extracted with EtOAc (3×). The combined organic layers were then washed with H₂O, 1N HCl (2×), saturated NaHCO₃, H₂O and brine before drying with Na₂SO₄. The material was filtered and concentrated under reduced pressure to give 0.1656 g of crude oil Purification of this material was not required. This aniline (0.1544 g, 0.55 mmol), benzoylpiperazine hydrochloride (0.1496 g, 0.66 mmol) and NaOtBu (0.1586 g, 1.65 mmol) were added to a tube. The tube was evacuated and flushed with argon for three cycles. Toluene (1.7 mL) and NMP (1.0 mL) were then added and the mixture was degassed with argon for 30 minutes. Pd₂(dba)₃ (0.0101 g, 0.011 mmol) and BINAP (0.0137 g, 0.022 mmol) were then added, the tube was sealed and heated overnight at 80° C. After cooling, the mixture was diluted with water and EtOAc. The aqueous layer was back-extracted with EtOAc (3×). The combined organic layers were then washed with H₂O, 1N HCl (2×), saturated NaHCO₃, H₂O and brine before drying with Na₂SO₄. The material was filtered and concentrated under reduced pressure to give a crude oil. Purification of this material via flash chromatography (20% EtOAc/hexane) gave 0.125 g (58% yield) of TRV 1166 (4-(3-(isopropylamino)-4-(trifluoromethyl)phenyl)piperazin-1-yl)(phenyl)methanone

¹H NMR (700 MHz, CDCl₃) δ=7.46-7.41 (m, 5H), 7.30 (d, J=8.4 Hz, 1H), 6.21 (dd, J=8.4, 2.1 Hz, 1H), 6.16 (s, 1H), 4.12-4.11 (m, 1H), 3.93 (br s, 2H), 3.68-3.66 (m, 1H), 3.59 (br s, 2H), 3.33 (br s, 2H), 3.18 (br s, 2H), 1.24 (d, J=6.2 Hz, 6H).

### Example 21

### Synthesis of TRV 1164

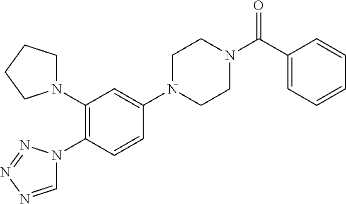 [SMILES: O=C(c1ccccc1)N1CCN(c2ccc(-n3cnnn3)c(N3CCCC3)c2)CC1]

### TRV 1164—phenyl(4-(3-(pyrrolidin-1-yl)-4-(1H-tetrazol-1-yl)phenyl)piperazin-1-yl)methanone

Scheme for TRV 1164

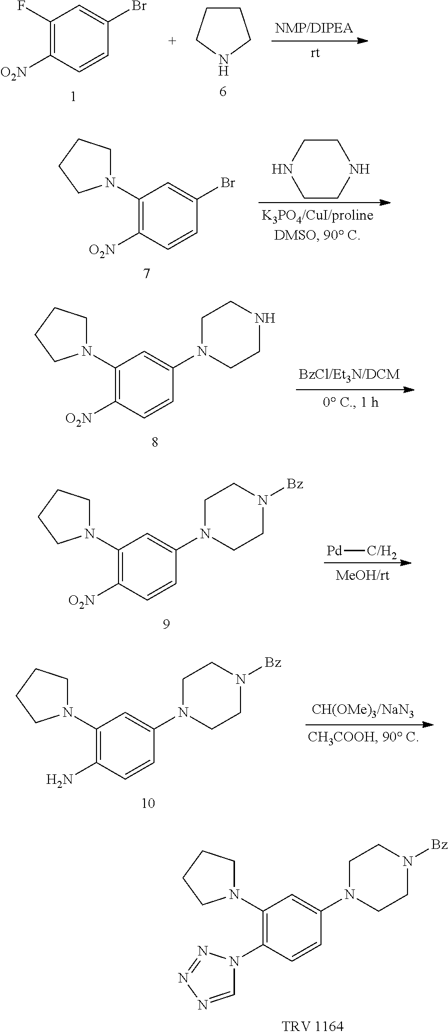

### (4-(3-(pyrrolidin-1-yl)-4-(1H-tetrazol-1-yl)phenyl)piperazin-1-yl)methanone (TRV 1164)

A 250 mL round-bottom flask was charged with a solution of (4-(4-amino-3-(pyrrolidin-1-yl)phenyl)piperazin-1-yl)(Phenyl)methanone 10 (1 g, 2.85 mmole) in AcOH (10 mL). To this was added NaN₃ (0.277 g, 4.27 mmole). To the mixture was added trimethoxymethane (0.452 g, 4.27 mmole) and the resulting solution was stirred for 4 hr at 90° C. The resulting mixture was concentrated under vacuum and diluted with 15 mL water and extracted with ethyl acetate (2×50 mL). Final purification was done by column chromatography to afford the title compound (4-(3-(pyrrolidin-1-yl)-4-(1H-tetrazol-1-yl)phenyl)piperazin-1-yl)methanone (TRV 1164)

(0.8 g, 72%).

### Example 22

### Synthesis of TRV 1163

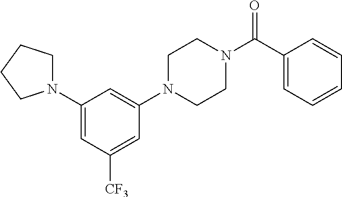 [SMILES: O=C(c1ccccc1)N1CCN(c2cc(N3CCCC3)cc(C(F)(F)F)c2)CC1]

### TRV 1163 phenyl(4-(3-(pyrrolidin-1-yl)-5-(trifluoromethyl)phenyl)piperazin-1-yl)methanone

Scheme for TRV 1163

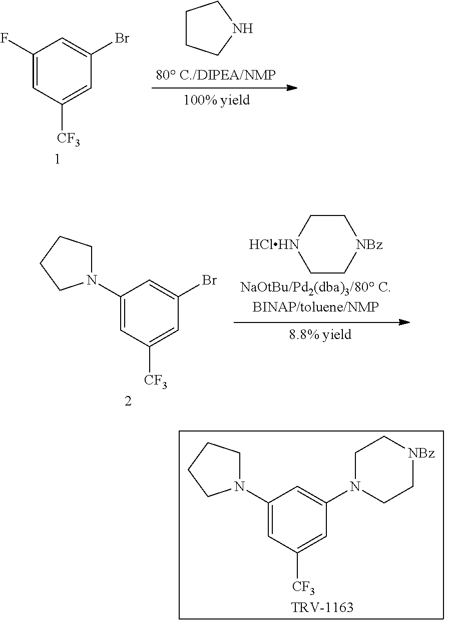

3-bromo-5-fluorobenzotrifluoride (0.7304 g, 3.0 mmol), pyrrolidine (0.30 mL, 3.6 mmol), DIPEA (0.78 mL, 4.5 mmol) and NMP (4 mL) were added to a tube. The tube was sealed and heated overnight at 80° C. After cooling, the reaction mixture was diluted with water and EtOAc. The aqueous layer was back-extracted with EtOAc (3×). The combined organic layers were then washed with H₂O, 1N HCl (2×), saturated NaHCO₃, H₂O and brine before drying with Na₂SO₄. The material was filtered and concentrated under reduced pressure to give 0.9 g of a crude oil. Purification of this material was not required. This aniline (0.7322 g, 2.49 mmol), benzoylpiperazine hydrochloride (0.6778 g, 2.99 mmol) and NaOtBu (0.7179 g, 7.47 mmol) were added to a tube. The tube was evacuated and flushed with argon for three cycles. Toluene (7.5 mL) and NMP (4.5 mL) were then added and the mixture was degassed with argon for 30 minutes. Pd₂(dba)₃ (0.0456 g, 0.0498 mmol) and BINAP (0.0620 g, 0.0996 mmol) were then added, the tube was sealed and heated overnight at 80° C. After cooling, the mixture was diluted with water and EtOAc. The aqueous layer was back-extracted with EtOAc (3×). The combined organic layers were then washed with H₂O, 1N HCl (2×), saturated NaHCO₃, H₂O and brine before drying with Na₂SO₄. The material was filtered and concentrated under reduced pressure to give a crude oil. Purification of this material via flash chromatography (35% EtOAc/hexane) gave 0.0881 g (8.8% yield) of TRV 1163, phenyl(4-(3-(pyrrolidin-1-yl)-5-(trifluoromethyl)phenyl)piperazin-1-yl)methanone. ¹H NMR (700 MHZ, CDCl₃) δ=7.43 (br s, 5H), 6.45 (s, 1H), 6.34 (s, 1H), 6.16 (s, 1H), 3.94 (br s, 2H), 3.59 (br s, 2H), 3.30-3.28 (m, 6H), 3.14 (br s, 2H), 2.02-2.01 (m, 4H).

### Example 23

### Synthesis of TRV 1162

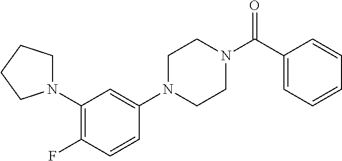 [SMILES: O=C(c1ccccc1)N1CCN(c2ccc(F)c(N3CCCC3)c2)CC1]

### TRV 1162—(4-(4-fluoro-3-(pyrrolidin-1-yl)phenyl)piperazin-1-yl)(phenyl)methanone

Scheme for TRV 1162

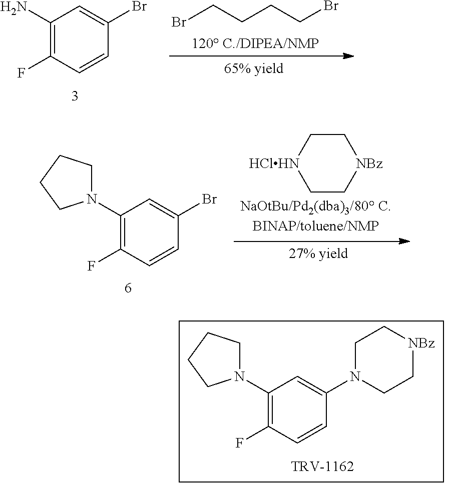

5-bromo-2-fluoroaniline (0.500 g, 2.63 mmol) and 1,4-dibromobutane (0.31 mL, 2.63 mmol) were combined with DIPEA (0.92 mL, 5.26 mmol) in NMP (3.0 mL) in a tube. The tube was sealed and heated to 120° C. After 2 hours the reaction had reached 95% conversion. It was diluted with water and EtOAc. The aqueous layer was back-extracted with EtOAc (3×). The combined organic layers were then washed with H₂O, 1N HCl (2×), saturated NaHCO₃, H₂O and brine before drying with Na₂SO₄. The material was filtered and concentrated under reduced pressure to give 1.0 g of crude red oil. This material was purified via flash chromatography (2% EtOAc/hexane) to give 0.4152 g (65% yield) of the pyrrolidinyl aniline. The pyrrolidinyl aniline (0.3351 g, 1.37 mmol), benzoylpiperazine hydrochloride (0.3741 g, 1.65 mmol) and NaOtBu (0.3950 g, 4.11 mmol) were added to a tube. The tube was evacuated and flushed with argon for three cycles. Toluene (4.2 mL) and NMP (2.5 mL) were then added and the mixture was degassed with argon for 30 minutes. Pd₂(dba)₃ (0.0251 g, 0.027 mmol) and BINAP (0.0341 g, 0.055 mmol) were then added, the tube was sealed and heated overnight at 80° C. After cooling, the mixture was diluted with water and EtOAc. The aqueous layer was back-extracted with EtOAc (3×). The combined organic layers were then washed with H₂O, 1N HCl (2×), saturated NaHCO₃, H₂O and brine before drying with Na₂SO₄. The material was filtered and concentrated under reduced pressure to give crude oil. This material was purified via flash chromatography (30% EtOAc/hexane) to give 0.1286 g (27% yield) of TRV 1162, (4-(4-fluoro-3-(pyrrolidin-1-yl)phenyl)piperazin-1-yl)(phenyl)methanone. ¹H NMR (700 MHz, CDCl₃) δ=7.44-7.41 (m, 5H), 6.88 (dd, J=13.3, 8.4 Hz, 1H), 6.24 (dd, J=7.7, 2.8 Hz, 1H), 6.20 (dt, J=8.4, 2.8 Hz, 1H), 3.94 (br s, 2H), 3.58 (br s, 2H), 3.38-3.36 (m, 4H), 3.17 (br s, 2H), 3.01 (br s, 2H), 1.96-1.82 (m, 4H).

### Example 24

### Synthesis of TRV 1160

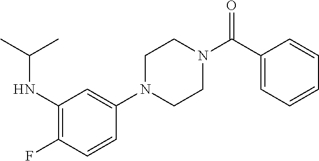 [SMILES: CC(C)Nc1cc(N2CCN(C(=O)c3ccccc3)CC2)ccc1F]

### TRV 1160—(4-(4-fluoro-3-(isopropylamino)phenyl)piperazin-1-yl)(phenyl)methanone

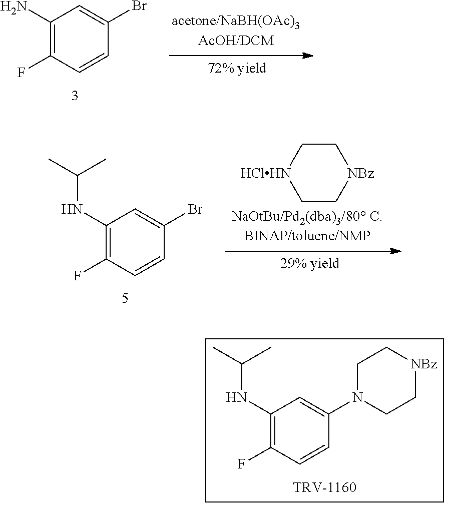

5-bromo-2-fluoroaniline (0.500 g, 2.63 mmol) and acetone (0.19 mL, 2.63 mmol) were dissolved in DCM (8.8 mL) and then treated with NaBH(OAc)₃ (0.837 g, 3.95 mmol) and AcOH (0.23 mL, 3.95 mmol). This mixture was stirred at room temperature under argon until complete by TLC. The reaction was quenched by the dropwise addition of. 1N NaOH (20 mL). This mixture was then extracted with EtOAc (3×20 mL). The combined organic layers were washed with H₂O, brine, dried (Na₂SO₄), filtered and concentrated to give 0.8 g of an oil. This crude oil was purified via flash chromatography (3% EtOAc/hexane to give 0.4422 g (72% yield) of the desired isopropyl aniline. The isopropyl aniline (0.3985 g, 1.72 mmol), benzoylpiperazine hydrochloride (0.467 g, 2.06 mmol) and NaOtBu (0.4959 g, 5.16 mmol) were added to a tube. The tube was evacuated and flushed with argon for three cycles. Toluene (5.2 mL) and NMP (3.1 mL) were then added and the mixture was degassed with argon for 30 minutes. Pd₂(dba)₃ (0.0321 g, 0.035 mmol) and BINAP (0.0436 g, 0.070 mmol) were then added, the tube was sealed and heated overnight at 100° C. After cooling, the mixture was diluted with water and EtOAc. The aqueous layer was back-extracted with EtOAc (3×). The combined organic layers were then washed with H₂O, 1N HCl (2×), saturated NaHCO₃, H₂O and brine before drying with Na₂SO₄. The material was filtered and concentrated under reduced pressure to give 1.6 g of crude oil. Initial purification via flash chromatography (45% EtOAc/hexane) failed to separate the product from the minor by-product. A second chromatographic process (25% EtOAc/hexane) was used to afford 0.0688 g (29% yield) of TRV 1160, (4-(4-fluoro-3-(isopropylamino)phenyl)piperazin-1-yl)phenyl)methanone. ¹H NMR (500 MHz, CDCl3) δ=7.49 (br s, 5H), 6.91 (dd, J=11.5, 9.0 Hz, 1H), 6.34 (dd, J=7.5, 2.5 Hz, 1H), 6.18 (dt, J=9.0, 2.5 Hz, 1H), 3.40 (br s, 2H), 3.76 (br s, 1H), 3.69-3.64 (m, 3H), 3.21 (br s, 2H), 3.06 (br s, 2H), 1.30 (d, J=4.0 Hz, 6H).

### Example 25

### Synthesis of TRV 1159

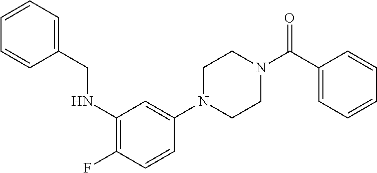 [SMILES: O=C(c1ccccc1)N1CCN(c2ccc(F)c(NCc3ccccc3)c2)CC1]

### TRV 1159 (4-(3-(benzylamino)-4-fluorophenyl)piperazin-1-yl)(phenyl)methanone

Scheme for TRV 1159

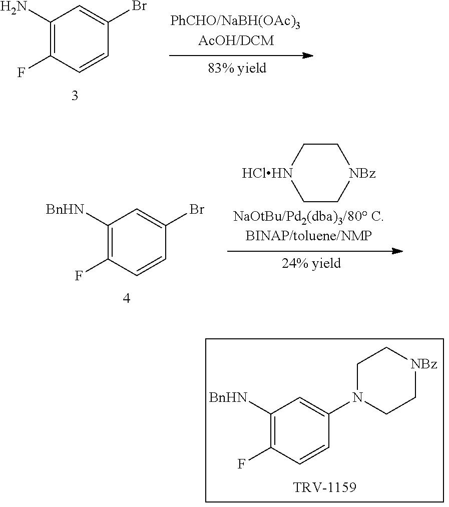

5-bromo-2-fluoroanile (0.500 g, 2.63 mmol) and benzaldehyde (0.27 mL, 2.63 mmol) were dissolved in DCM (8.8 mL) and then treated with NaBH(OAc)₃ (0.837 g, 3.95 mmol) and AcOH (0.23 mL, 3.95 mmol). This mixture was stirred at room temperature under argon until complete by TLC. The reaction was quenched by the dropwise addition of 1N NaOH (20 mL). This mixture was then extracted with EtOAc (3×20 mL). The combined organic layers were washed with H₂O, brine, dried (Na₂SO₄), filtered and concentrated to give 1.0 g of an oil. This crude oil was purified via flash chromatography (5% EtOAc/hexane to give 0.6127 g (83% yield) of the desired benzylamine. The benzyl amine (0.5032 g, 1.8 mmol), benzoylpiperazine hydrochloride (0.4897 g, 2.16 mmol) and NaOtBu (0.517 g, 5.38 mmol) were added to a tube. The tube was evacuated and flushed with argon for three cycles. Toluene (5.4 mL) and NMP (3.2 mL) were then added and the mixture was degassed with argon for 30 minutes. Pd₂(dba)₃ (0.033 g, 0.036 mmol) and BINAP (0.0448 g, 0.072 mmol) were then added, the tube was sealed and heated overnight at 100° C. After cooling, the mixture was diluted with water and EtOAc. The aqueous layer was back-extracted with EtOAc (3×). The combined organic layers were then washed with H₂O, 1N HCl (2×), saturated NaHCO₃, H₂O and brine before drying with Na₂SO₄. The material was filtered and concentrated under reduced pressure to give 1.3 g of crude brown oil. Initial purification via flash chromatography (45% EtOAc/hexane) failed to separate the product from the minor by-product. A second chromatographic process (20% EtOAc/hexane) was used to afford 0.1998 g (24% yield) of TRV 1159. ¹H NMR (500 MHz, CDCl3) δ=7.47-7.41 (m, 5H), 7.38-7.34 (m, 4H), 7.30-7.27 (m, 1H), 6.88 (dd, J=11.5, 8.5 Hz, 1H), 6.26 (dd, J=7.5, 3.0 Hz, 1H), 6.16 (dt, J=8.5, 3.0 Hz, 1H), 4.35 (s, 2H), 4.32 (br s, 1H), 3.89 (br s, 2H), 3.54 (br s, 2H), 3.09 (br s, 2H), 2.95 (br s, 2H).

### Example 26

### Synthesis of TRV 1158

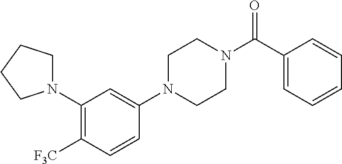 [SMILES: O=C(c1ccccc1)N1CCN(c2ccc(C(F)(F)F)c(N3CCCC3)c2)CC1]

### TRV 1158—phenyl(4-(3-(pyrrolidin-1-yl)-4-(trifluoromethyl)phenyl)piperazin-1-yl)methanone

Scheme for TRV 1158

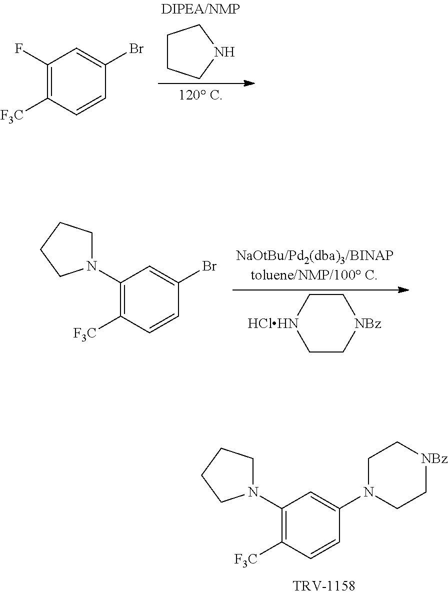

A mixture of 4-bromo-2-fluorobenzotrifluoride (1.511 g, 6.2 mmol), pyrrolidine (0.62 mL, 7.46 mmol), DIPEA (1.6 mL, 9.33 mmol) and NMP (8 mL) were sealed in a tube and heated to 120° C. overnight. The solution was cooled to room temperature and diluted with water and EtOAc. The layers were separated and the aqueous layer was back-extracted with EtOAc (3×). The combined organic layers were then washed successively with H₂O, 1N HCl(aq), saturated NaHCO₃(aq), H₂O, and brine before drying with Na₂SO₄. The mixture was filtered and concentrated to give a crude oil, which was purified via flash chromatography (5% EtOAc/hexane) to give 0.9667 g (53% yield) of desired product. This bromo-intermediate (0.5312 g, 1.8 mmol), benzoylpiperazine hydrochloride (0.4987 g, 2.2 mmol) and NaOtBu (0.5189 g, 5.4 mmol) were charged to a flask which was subsequently purged and evacuated with argon (3 cycles). Toluene (5.4 mL) and NMP (3.2 mL) were then added and the solution was degassed for 30 minutes before added Pd₂(dba)₃ (0.033 g, 0.036 mmol) and BINAP (0.045 g, 0.072 mmol) all at once. The flask was then heated to 100° C. overnight under argon. The mixture was cooled to room temperature and diluted with EtOAC before filtering through Celite. The organic layer was then washed successively with H₂O, 1N HCl(aq), saturated NaHCO₃(aq), H₂O, and brine before drying with Na₂SO₄. The mixture was filtered and concentrated to give 0.5081 g of crude oil. The oil was purified via flash chromatography (45% EtOAc/hexane) to give another crude oil that was slightly impure. This oil was crystallized from EtOAc (solvent) and hexane (anti-solvent) to give 0.101 g of white crystals of TRV 1158, phenyl(4-(3-(pyrrolidin-1-yl)-4-(trifluoromethyl)phenyl)piperazin-1-yl)methanone. ¹H NMR (500 MHz, DMSO) S=7.47-7.41 (m, 5H), 7.35 (d, J=8.5 Hz, 1H), 6.49 (dd, J=8.5, 1.5 Hz, 1H), 6.46 (m, 1H), 3.72 (br s, 2H), 3.45 (br s, 2H), 3.30 (br s, 4H), 3.23-3.20 (m, 4H), 1.88-1.83 (m, 4H).

### Example 27

### Synthesis of TRV 1156

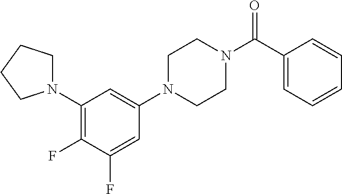 [SMILES: O=C(c1ccccc1)N1CCN(c2cc(F)c(F)c(N3CCCC3)c2)CC1]

### TRV 1156—(4-(3,4-difluoro-5-(pyrrolidin-1-yl)phenyl)piperazin-1-yl)(phenyl)methanone

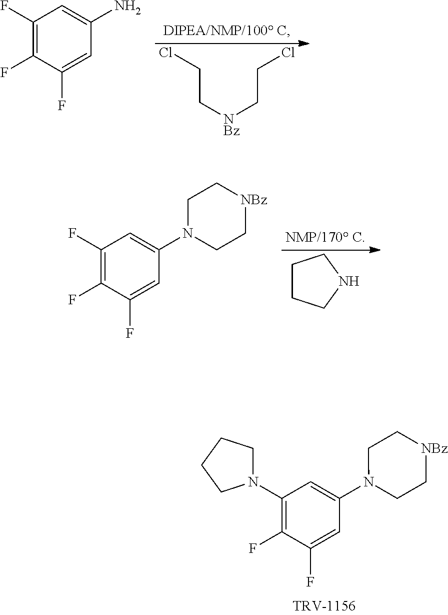

A mixture of 3,4,5-trifluoroaniline (0.500 g, 3.4 mmol), Bis(2-chloroethyl)benzoyl amine (0.911 g, 3.7 mmol), DIPEA (1.5 mL, 8.5 mmol) in NMP (6.8 mL) were scaled in a tube and heated to 115° C. overnight. The red solution was cooled to room temperature and diluted with water and EtOAc. The layers were separated and the aqueous layer was back-extracted with EtOAc (3×). The combined organic layers were then washed successively with H₂O, 1N HCl(aq), saturated NaHCO₃(aq), H₂O, and brine before drying with Na₂SO₄. The mixture was filtered and concentrated to give a crude oil, which was purified via flash chromatography (0,5,10,15 . . . 40% EtOAc/Hexane gradient) to give 0.466 g (43% yield) of the piperazine. This material (0.201 g, 0.63 mmol) was added to a sealed tube along with pyrrolidine (0.18 mL, 2.2 mmol) and NMP (1.5 mL) and heated to 170° C. overnight. The reaction was then cooled to room temperature and diluted with water and EtOAc. The layers were separated and the aqueous layer was back-extracted with EtOAc (3×). The combined organic layers were then washed successively with H₂O, 1N HCl(aq), saturated NaHCO₃(aq), H₂O, and brine before drying with Na₂SO₄. The mixture was filtered and concentrated to give a crude oil, which was purified via recrystallization from EtOAc (solvent) and hexane (anti-solvent) to afford 0.1755 g (75% yield) of yellow needles which were confirmed to be (4-(3,4-difluoro-5-(pyrrolidin-1-yl)phenyl)piperazin-1-yl)phenyl)methanone. ¹H NMR (700 mHZ, DMSO) δ=7.49-7.42 (m, 5H), 6.27-6.24 (m, 1H), 5.99 (d, J=6.3 Hz, 1H), 3.73 (br s, 2H), 3.46 (br s, 2H), 3.34-3.33 (m, 4H), 3.17 (br s, 2H), 3.06 (br s, 2H), 1.90-1.87 (m, 4H).

### Example 28

### Synthesis of 1155

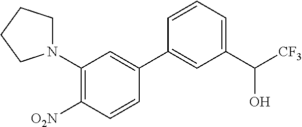 [SMILES: O=[N+]([O-])c1ccc(-c2cccc(C(O)C(F)(F)F)c2)cc1N1CCCC1]

### TRV 1155—2,2,2-trifluoro-1-(4′-nitro-3′-(pyrrolidin-1-yl)-[1,1′-biphenyl]-3-yl)ethanol

Scheme for TRV 1155

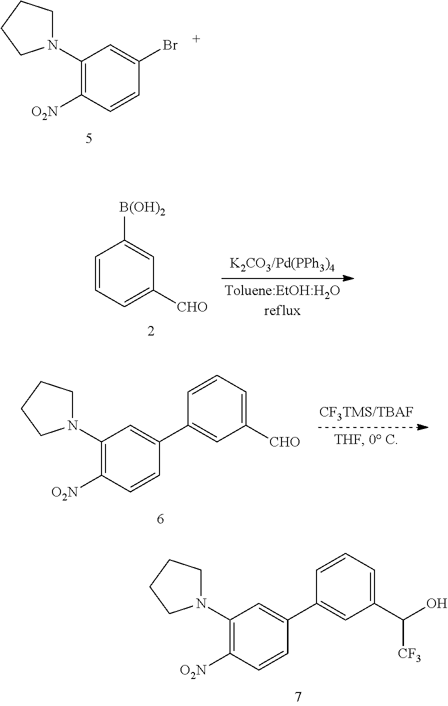 [SMILES: O=[N+]([O-])c1ccc(Br)cc1N1CCCC1 ; O=Cc1cccc(B(O)O)c1 ; O=Cc1cccc(-c2ccc([N+](=O)[O-])c(N3CCCC3)c2)c1 ; O=[N+]([O-])c1ccc(-c2cccc(C(O)C(F)(F)F)c2)cc1N1CCCC1]

### Example 29

### Synthesis of TRV 1140 and TRV 1153

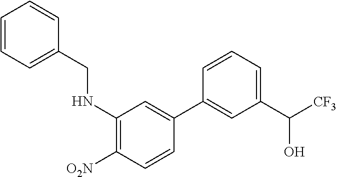 [SMILES: O=[N+]([O-])c1ccc(-c2cccc(C(O)C(F)(F)F)c2)cc1NCc1ccccc1]

### TRV 1140—1-(3′-(benzylamino)-4′-nitro-[1,1′-biphenyl]-3-yl)-2,2,2-trifluoroethanol

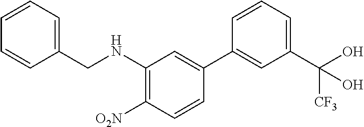 [SMILES: O=[N+]([O-])c1ccc(-c2cccc(C(O)(O)C(F)(F)F)c2)cc1NCc1ccccc1]

### TRV 1153—1-(3′-(benzylamino)-4′-nitro-[1,1′-biphenyl]-3-yl)-2,2,2-trifluoroethane-1,1-diol

Scheme for synthesis of TRV 1140 and TRV 1153

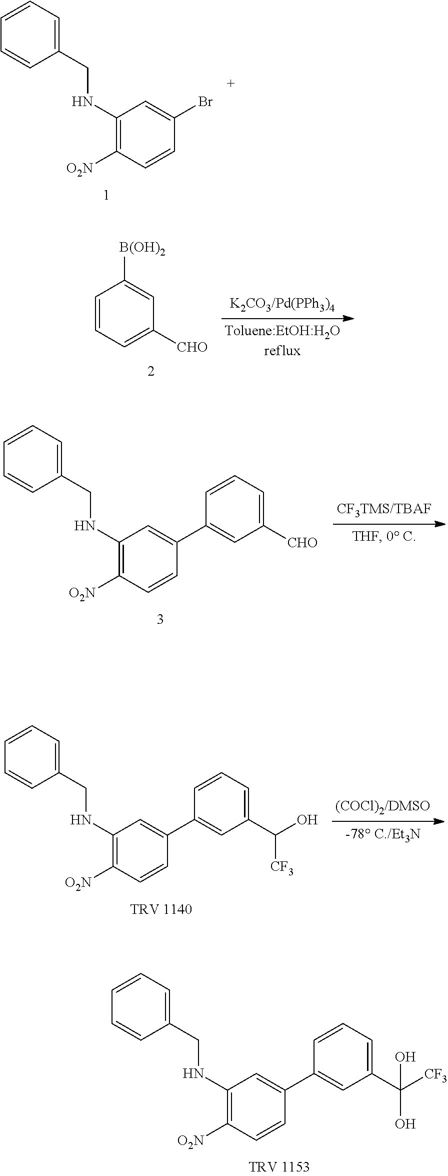 [SMILES: O=[N+]([O-])c1ccc(Br)cc1NCc1ccccc1 ; O=Cc1cccc(B(O)O)c1 ; O=Cc1cccc(-c2ccc([N+](=O)[O-])c(NCc3ccccc3)c2)c1 ; O=[N+]([O-])c1ccc(-c2cccc(C(O)C(F)(F)F)c2)cc1NCc1ccccc1 ; O=[N+]([O-])c1ccc(-c2cccc(C(O)(O)C(F)(F)F)c2)cc1NCc1ccccc1]

Synthesis of 3

To a solution of N-benzyl-5-bromo-2-nitrobenzenamine (Ig, 3.2 mmol) and 3-formylphenylboronic acid (0.58 g, 3.92 mmol) in Toluene: EtOH: H₂O (8:8:1) was added K₂CO₃ (1.4 g, 9.78 mmol). After stirring reaction for 15 min under argon tetrakis(triphicnylhosphine) palladium (0.184 mg, 0.016 mol) was added, resulting mixture was then heated at 100° C. for 12 h. The reaction mixture was cooled to room temperature and diluted with ethyl acetate and extracted three times with water. The aqueous layer was then acidified with 2M HCl to get 3′-(benzyl amino)-4′-nitrophenyl-3-carbaldehyde (1.1 g), 90% yield.

¹H NMR (CDCl₃, 500 MHz): δ 4.67 (d, J=5 Hz, 2H), 6.93 (dd, J=5, 10 Hz, 1H), 7.02 (d, J=5 Hz, 1H), 7.39 (m, 1H), 7.45 (m, 4H), 7.68 (t, J=5, 10 Hz, 1H), 7.82-7.84 (m, 1H), 8.11-8.13 (m, 1H), 8.17 (m, 1H), 8.35 (d, J=100 Hz, 1H), 8.61 (bs, 1H)

Synthesis of TRV 1140

A mixture of aldehyde 3 (1.2 g, 3.6 mmol) and TMSCF₃ (0.615 g, 4.3 mmol) in 10 mL of THF cooled to 0° C. was treated with a catalytic amount (ca. 20 mg) of TBAF. Instantaneously, a yellow color developed with the initial evolution of fluorotrimethylsilane, and the reaction mixture was brought to ambient temperature and stirred. The mixture was periodically analyzed by TLC for the completion of the reaction. The resulting siloxy compounds were then hydrolyzed with aqueous HCl. After the reaction, the mixture was extracted with ether (75 mL), and the ether extracts were washed with water (50 mL) and brine (50 mL), dried over Na₂SO₄, and concentrated. The residue was purified by flash column chromatography to give TRV 1140, 1-(3′-(benzylamino)-4′-nitro-[1,1′-biphenyl]-3-yl)-2,2,2-trifluoroethanol, yield (0.7 g, 60%)

¹H NMR (CDCl₃, 500 MHz): δ 4.75 (d, J=5 Hz, 2H), 5.20-5.26 (m, 1H), 6.94 (d, J=5 Hz, 1H), 6.98 (dd, J=5, 10 Hz, 1H), 7.12 (d, J=2 Hz, 1H), 7.28 (t, J=5, 10 Hz, 1H), 7.37 (t, J=5, 10 Hz, 2H), 7.45 (d, J=5 Hz, 2H), 7.49-7.62 (m, 3H), 7.70 (bs, 1H), 8.19 (d, J=8.9 Hz, 1H), 8.80 (t, J=5.9 Hz, 1H)

Synthesis of TRV 1153

To a stirring solution of oxalyl chloride (3.0 mmol, 0.23 mL) in 2.0 mL of CH₂Cl₂, cooled to −78° C., was added drop wise 0.4 mL (5.5 mmol) of DMSO. After 2 min, a solution of 4 (0.1 g, 2.5 mmol) in 2.0 mL of CH₂Cl₂ was added dropwise over 5 min. After an additional 15 min, (7.5 mmol, 0.7 mL) of triethylamine was added, and the mixture was allowed to stir at −78° C. for 5 min and then warm to room temperature. The mixture was passed through a plug of 230-400-mesh silica gel, eluting with 25% ethyl acetate in hexanes, and the eluent was concentrated to give yellow oil. The oil was purified by flash chromatography on, eluting with 15% ethyl acetate in hexanes, to give 80 mg (83%) of TRV 1153, 1-(3′-(benzylamino)-4′-nitro-[1,1′-biphenyl]-3-yl)-2,2,2-trifluoroethane-1,1-diol, as an orange solid. ¹H NMR (CDCl₃, 500 MHz): δ 4.67 (d, J=5 Hz, 2H), 6.95 (dd, J=1.65, 8.8 Hz, 1H), 7.05 (d, J=1.8 Hz, 1H), 7.35-7.45 (m, 4H), 7.65 (t, J=7.65 Hz, 1H), 7.75 (m, 1H), 7.95 (m, 1H), 7.99 (m, 1H), 8.33 (d, J=8.85, 1H), 8.58 (m, 1H), 10.01 (bs, 1H)

### Example 30

### Synthesis of amino substituted analogs

General Scheme for Amino Substituted Analogs

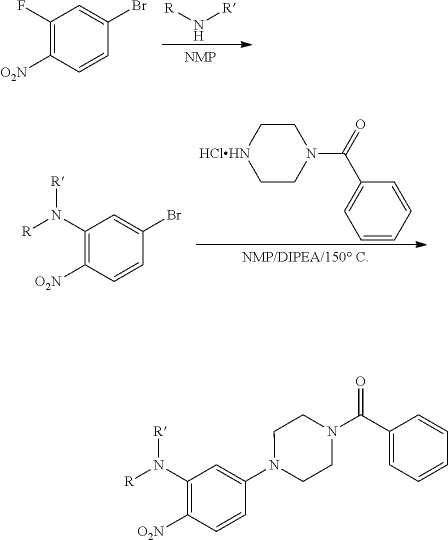

### Example 31

### Synthesis of TRV 1144 and TRV 1152

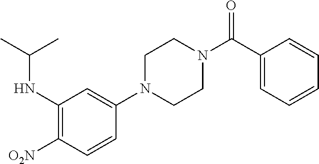 [SMILES: CC(C)Nc1cc(N2CCN(C(=O)c3ccccc3)CC2)ccc1[N+](=O)[O-]]

### TRV 1144—4-(3-(isopropylamino)-4-nitrophenyl)piperazin-1-yl)(phenyl)methanone

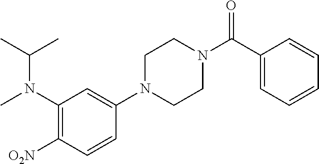 [SMILES: CC(C)N(C)c1cc(N2CCN(C(=O)c3ccccc3)CC2)ccc1[N+](=O)[O-]]

### TRV 1152—(4-(3-(isopropyl(methyl)amino)-4-nitrophenyl)piperazin-1-yl)(phenyl)methanone

4-Bromo-2-fluoro-1-nitrobenzene (2.00 g, 9.09 mmol) was massed into a sealed tube. The solid was dissolved in NMP (10 mL), isopropyl amine (1.17 mL, 13.6 mmol) was added and the tube was flushed with argon before sealing and stirring overnight at room temperature. The reaction was quenched by the addition of water. The mixture was extracted with MTBE (3×). The combined organic layers were washed with H₂O, 1N HCl(aq), H₂O (3×), brine, dried with Na₂SO₄, filtered and concentrated under reduced pressure to afford 2.30 g (98% yield) of orange oil. This material (1.09 g, 4.2 mmol) was dissolved in NMP (8 mL) and DIPEA (2.9 mL, 16.8 mmol) and benzoylpiperazine hydrochloride (1.90 g, 8.4 mmol) were added. The tube was flushed with argon before sealing and the reaction was heated to 150° C. overnight. The reaction was diluted with EtOAc and water. The layers were separated and the aqueous layer was back-extracted with EtOAc (3×). The combined organic layers were then washed with H₂O, 1N HCl(aq), H₂O (3×), brine, dried with Na₂SO₄, filtered and concentrated to afford 1.69 g of crude solid. This material was recrystallized from ethyl acetate (solvent) and hexane (anti-solvent) to provide 1.39 g of orange solid, TRV 1144, (4-(3-(isopropylamino)-4-nitrophenyl)piperazin-1-yl)(phenyl)methanone in 90% yield. ¹H NMR (500 MHz, CDCl3) δ=8.26 (d, J=7.7 Hz, 1H), 7.93 (d, J=9.8 Hz, 1H), 7.50-7.45 (m, 5H), 6.41 (dd, J=9.8, 2.4 Hz, 1H), 6.04 (d, J=2.4 Hz, 1H), 3.98-3.91 (m, 1H), 3.74-3.49 (m, 8H), 1.25 (d, J=6.3, 6H).

Sodium hydride (80.0 mg, 2.0 mmol) was suspended in DMF (5 mL) and cooled to 0° C. TRV 1144, (4-(3-(isopropylamino)-4-nitrophenyl)piperazin-1-yl)(phenyl)methanone (0.50 g, 1.36 mmol) dissolved in DMF (5 mL) was added to the above suspension at 0° C. The reaction was removed from the ice bath and warmed to room temperature. Methyl iodide (0.42 mL, 6.8 mmol) was then added and the reaction was heated to 65° C. and stirred overnight. The reaction was quenched with the dropwise addition of water and then diluted with EtOAc. The layers were separated and the aqueous layer was back-extracted. The combined organic layers were washed with water (3×), brine, dried (MgSO₄), filtered and concentrated to afford an orange solid. This solid was recrystallized from EtOAc (solvent) and hexane (anti-solvent) to afford orange crystals, which were filtered off. The mother liquor was then concentrated at ambient pressure and room temperature over several days, which afforded a second crop of crystals containing all desired compound. Obtained 43 mg (8.3% yield) of compound TRV 1152—(4-(3-(isopropyl(methyl)amino)-4-nitrophenyl)piperazin-1-yl)(phenyl)methanone

¹H NMR (500 MHz, DMSO) δ=7.81 (d, J=9.5 Hz, 1H), 7.50-7.45 (m, 5H), 6.50 (dd, J=9.5, 2.5 Hz, 1H), 6.36 (d, J=2.5 Hz, 1H), 3.75 (br s, 2H), 3.56-3.42 (m, 7H), 2.62 (s, 3H), 1.12 (d, J=6.5 Hz, 6H).

### Example 32

### Synthesis of TRV 1149 and TRV 1154

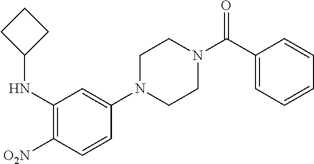 [SMILES: O=C(c1ccccc1)N1CCN(c2ccc([N+](=O)[O-])c(NC3CCC3)c2)CC1]

### TRV 1149—(4-(3-(cyclobutylamino)-4-nitrophenyl)piperazin-1-yl)(phenyl)methanone

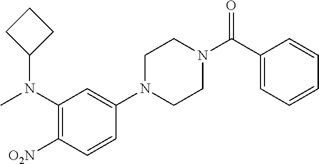 [SMILES: CN(c1cc(N2CCN(C(=O)c3ccccc3)CC2)ccc1[N+](=O)[O-])C1CCC1]

### TRV 1154—(4-(3-(cyclobutyl(methyl)amino)-4-nitrophenyl)piperazin-1-yl)(phenyl)methanone

In a manner similar to the synthesis of TRV 1144, (4-(3-(isopropylamino)-4-nitrophenyl)piperazin-1-yl)(phenyl)methanone 4-Bromo-2-fluoro-1-nitrobenzene (1.50 g, 6.82 mmol) was reacted with the cyclobutylamine (0.87 mL, 10.2 mmol) in NMP (8 mL) to provide 1.83 g of an orange solid (99% yield). This material (0.81 g, 2.98 mmol) was then dissolved in NMP (6 mL) and DIPEA (2.1 mL, 11.9 mmol) and reacted in a similar manner as described in the synthesis of TRV 1144, (4-(3-(isopropylamino)-4-nitrophenyl)piperazn-1-yl)(phenyl)methanone with benzolypiperazine hydrochloride (1.36 g, 5.98 mmol) to afford the desired material in crude form. Recrystallization from ethyl acetate (solvent) and hexane (antisolvent) afforded 0.78 g (69% yield) of yellow crystalline solid, TRV 1149, (4-(3-(cyclobutylamino)-4-nitrophenyl)piperazn-1-yl)phenyl)methanone ¹H NMR (500 MHz, DMSO) δ=8.34 (d, J=5.9 Hz, 1H), 7.92 (d, J=9.8 Hz, 1H), 7.50-7.45 (m, 5H), 6.43 (dd, J=9.8, 2.6 Hz, 1H), 5.87 (d, J=2.6 Hz, 1H), 4.17-4.13 (m, 1H), 3.74-3.48 (m, 8H), 2.50-2.43 (m, 2H), 2.00-1.91 (m, 2H), 1.81-1.74 (m, 2H). The yellow solid (0.517 g, 1.36 mmol) in DMF (5 mL) was added dropwise to a suspension, of sodium hydride (80 mg, 2.0 mmol) in DMF (5 mL) producing a dark red solution. This mixture was stirred at 0° C. for 5 minutes then warmed to r.t. MeI (0.42 mL, 6.8 mmol) was added and the mixture was heated to 65° C. overnight. The reaction was then cooled to r.t. and carefully quenched with water before diluting with ethyl acetate. The layers were separated and the organic layer was washed with water, brine, dried with Na₂SO₄, filtered and concentrated to give a 4:1 mixture of product to starting material, respectively. This mixture was separated with a semi-preparatory HPLC column to afford 0.0257 g (4.8% yield) of TRV 1154—(4-(3-(cyclobutyl(methyl)amino)-4-nitrophenyl)piperazin-1-yl)(phenyl)methanone

¹H NMR (500 MHz, CDCl3) δ=8.00 (d, J=9.5 Hz, 1H), 7.52-7.49 (m, 5H), 6.39 (dd, J=9.5, 2.5 Hz, 1H), 6.18 (d, J=2.5 Hz, 1H), 3.97-3.91 (m, 3H), 3.67 (br s, 2H), 3.49 (br s, 2H), 3.35 (br s, 2H), 2.81 (s, 3H), 2.27-2.22 (m, 2H), 2.19-2.10 (m, 2H), 1.77-1.71 (m, 2H).

### Example 33

### Synthesis of TRV 1146

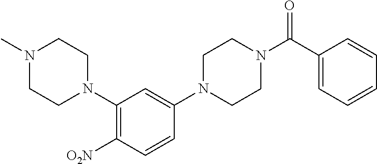 [SMILES: CN1CCN(c2cc(N3CCN(C(=O)c4ccccc4)CC3)ccc2[N+](=O)[O-])CC1]

### TRV 1146—(4-(3-(4-methylpiperazin-1-yl)-4-nitrophenyl)piperazin-1-yl)(phenyl)methanone

In a manner similar to the synthesis of TRV 1144, (4-(3-(isopropylamino)-4-nitrophenyl)piperazin-1-yl)(phenyl)methanone 4-Bromo-2-fluoro-1-nitrobenzene (1.00 g, 4.54 mmol) was first reacted with the 1-methylpiperazine (0.76 mL, 6.81 mmol) to provide 1.26 g of orange oil (92% yield). This material (0.51 g, 1.7 mmol) was dissolved in NMP (5 mL) and DIPEA (1.2 mL, 6.8 mmol) and reacted in a similar manner as described in the synthesis of TRV 1144, (4-(3-(isopropylamino)-4-nitrophenyl)piperazin-1-yl)(phenyl)methanone with benzolpiperazine hydrochloride (0.771 g, 3.4 mmol) to afford the desired material in crude form. Purification with flash chromatography (5% MeOH/DCM) afforded 0.39 g (56% yield) of fluffy yellow solid TRV 1146—(4-(3-(4-methylpiperazin-1-yl)-4-nitrophenyl)piperazin-1-yl)(phenyl)methanone.

¹H NMR (500 MHz, DMSO) δ=7.91 (d, J=9.4 Hz, 1H), 7.50-7.44 (m, 5H), 6.59 (dd, J=9.4, 2.6 Hz, 1H), 6.41 (d, J=2.6 Hz, 1H), 3.74-3.47 (m, 8H), 3.0 (t, J=4.6 Hz, 4H), 2.46-2.44 (m, 4H), 2.23 (s, 3H).

### Example 34

### Synthesis of TRV 1145

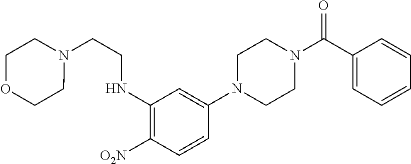 [SMILES: O=C(c1ccccc1)N1CCN(c2ccc([N+](=O)[O-])c(NCCN3CCOCC3)c2)CC1]

### TRV 1145—(4-(3-((2-morpholinoethyl)amino)-4-nitrophenyl)piperazin-1-ylX)(phenyl)methanone

In a manner similar to the synthesis of TRV 1144, (4-(3-(isopropylamino)-4-nitrophenyl)piperazin-1-yl)(phenyl)methanone 4-Bromo-2-fluoro-1-nitrobenzene (2.00 g, 9.09 mmol) was first reacted with 4-(2-aminoethyl)morpholine (1.8 mL, 13.6 mmol) to provide 2.93 g of orange solid (98% yield). This material (1.00 g, 3.03 mmol) was dissolved in NMP (8 mL) and DIPEA (2.1 mL (12.1 mmol) and reacted in a similar manner as described in the synthesis of TRV 1144, (4-(3-(isopropylamino)-4-nitrophenyl)piperazin-1-yl)(phenyl)methanone with benzolpiperazine hydrochloride (1.37 g, 6.06 mmol) to afford the desired material in crude form. Purification with flash chromatography (2% MeOH/DCM) afforded 0.58 g (44% yield) of TRV 1145—(4-(3-((2-morpholinoethyl)amino)-4-nitrophenyl)piperazin-1-yl)(phenyl)methanone.

¹H NMR (500 MHz, DMSO) δ=8.64 (t, J=4.5 Hz, 1H), 7.93 (d, J=9.8 Hz, 1H), 7.50-7.45 (m, 5H), 6.41 (dd, J=9.8, 2.5 Hz, 1H), 6.03 (d, J=2.5 Hz, 1H), 3.74-3.49 (m, 8H), 3.61-3.59 (m, 4H), 3.40-3.37 (m, 2H), 2.63 (t, J=6.1 Hz, 2H), 2.44 (m, 4H).

### Example 35

### Synthesis of TRV 1142 and TRV 1143

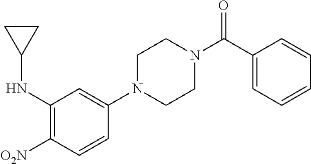 [SMILES: O=C(c1ccccc1)N1CCN(c2ccc([N+](=O)[O-])c(NC3CC3)c2)CC1]

### TRV 1142—(4-(3-(cyclopropylamino)-4-nitrophenyl)piperazin-1-yl)(phenyl)methanone

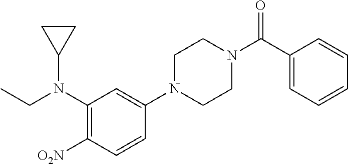 [SMILES: CCN(c1cc(N2CCN(C(=O)c3ccccc3)CC2)ccc1[N+](=O)[O-])C1CC1]

### TRV 1143—(4-(3-(cyclopropyl(methyl)amino)-4-nitrophenyl)piperazin-1-yl)(phenyl)methanone

In a manner similar to the synthesis of TRV 1144, (4-(3-(isopropylamino)-4-nitrophenyl)piperazin-1-yl)(phenyl)methanone 4-Bromo-2-fluoro-1-nitrobenzene (2.00 g, 9.09 mmol) was dissolved in NMP (10 mL) and reacted with cyclopropylamine (0.96 mL, 13.6 mmol) to afford 2.29 of bright yellow solid (98% yield). This material (1.00 g, 3.89 mmol) was dissolved in NMP (8 mL) and DIPEA (2.7 mL, 15.6 mmol) and reacted in a similar manner as described in the synthesis of TRV 1144, (4-(3-(isopropylamino)-4-nitrophenyl)piperazin-1-yl)(phenyl)methanone with benzoylpiperazine hydrochloride (1.76 g, 7.78 mmol) to afford the desired material in crude form. Approximately 0.5 g of this material was subjected to recrystallization using EtOAc (solvent) and hexane (anti-solvent) and obtained 0.34 g of yellow needles of TRV 1142, (4-(3-(cyclopropylamino)-4-nitrophenyl)piperazin-1-yl)(phenyl)methanone. ¹H NMR (500 MHz, DMSO) δ=8.26 (s, 1H), 7.91 (d, J=9.5 Hz, 1H), 7.48-7.43 (m, 5H), 6.45 (dd, J=9.5, 2.6 Hz, 1H), 6.41 (d, J=2.6 Hz, 1H), 3.74-3.49 (m, 8H), 2.61-2.58 (m, 1H), 0.88-0.84 (m, 2H), 0.59-0.56 (m, 2H). Sodium hydride (0.072 g, 1.8 mmol) was suspended in DMF (10 mL) and cooled to 0° C. TRV 1142—(4-(3-(cyclopropylamino)-4-nitrophenyl)piperazin-1-yl)(phenyl)methanone (0.500 g, 1.36 mmol) was dissolved in DMF (5 mL) and was added to the sodium hydride suspension via an addition funnel. The resultant deep red solution was stirred for 60 minutes at 0° C. Ethyl iodide (0.16 mL, 2.0 mmol) was then added at 0° C., the reaction was removed from the ice bath, and stirred overnight at room temperature. The reaction was quenched with water and diluted with MTBE. The layers were separated and then the aqueous layer was back-extracted with MTBE. The combined organic layers were washed with H₂O (3×), brine, dried with Na₂SO₄, filtered and concentrated under reduced pressure to afford 0.600 g of yellow oil. This material was purified with flash chromatography (5% gradient from 0 to 35% ethyl acetate/hexane) to produce 0.22 g (41% yield) of a yellow solid, TRV 1143, (4-(3-(cyclopropyl(methyl)amino)-4-nitrophenyl)piperazin-1-yl)(phenyl)methanone. ¹H NMR (500 MHz, DMSO) δ=7.75 (d, J=9.3 Hz, 1H), 7.50-7.44 (m, 5H), 6.55 (d, J=2.6 Hz, 1H), 6.52 (dd, J=9.3, 2.6 Hz, 1H), 3.75-3.34 (m, 8H), 3.23 (q, J=7.1 Hz, 2H), 2.71-2.67 (m, 1H), 1.13 (t, J=7.1 Hz, 3H), 0.73-0.69 (m, 2H), 0.37-0.34 (m, 2H).

### Example 36

### Synthesis of TRV 1141

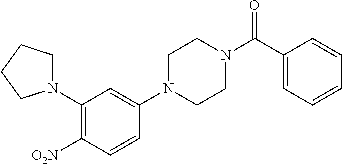 [SMILES: O=C(c1ccccc1)N1CCN(c2ccc([N+](=O)[O-])c(N3CCCC3)c2)CC1]

### TRV 1141—(4-(4-nitro-3-(pyrrolidin-1-yl-yl)(phenyl)methanone

In a manner similar to the synthesis of TRV 1144, (4-(3-(isopropylamino)-4-nitrophenyl)piperazin-1-yl)(phenyl)methanone 4-Bromo-2-fluoro-1-nitrobenzene (1.00 g, 4.54 mmol) was first reacted with pyrrolidine (0.56 mL, 6.81 mmol) to provide 1.20 g of an orange solid (97% yield). This material (1.18 g, 4.35 mmol) was dissolved in NMP (10 mL) and DIPEA (1.52 mL, 8.7 mmol) and reacted in a similar manner as described in the synthesis of TRV 1144, (4-(3-(isopropylamino)-4-nitrophenyl)piperazin-1-yl)(phenyl)methanone with benzolpiperazine hydrochloride (1.97 g, 8.7 mmol) to afford the desired material in crude form. Purification with flash chromatography (50% EtOAc/hexane) afforded 0.19 g (11% yield) of TRV 1141—(4-(4-nitro-3-(pyrrolidin-1-yl)phenyl)piperazin-1-yl)(phenyl)methanone. Further purification from EtOAc/Hexane resulted in 0.145 g of TRV 1141—(4-(4-nitro-3-(pyrrolidin-1-yl)phenyl)piperazin-1-yl)(phenyl)methanone as orange needles.

¹H NMR (500 MHz, DMSO) δ=7.73 (d, J=9.4 Hz, 1H), 7.50-7.44 (m, 5H), 6.43 (dd, J=9.4, 2.4 Hz, 1H), 6.20 (d, J=2.4 Hz, 1H), 3.74-3.36 (m, 8H), 3.15-3.13 (M, 4H), 1.91-1.89 (M, 4H).

### Example 37

### Synthesis of TRV 1139

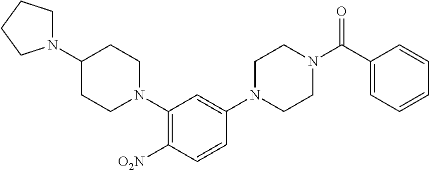 [SMILES: O=C(c1ccccc1)N1CCN(c2ccc([N+](=O)[O-])c(N3CCC(N4CCCC4)CC3)c2)CC1]

### TRV 1139—(4-(4-nitro-3-(4-(pyrrolidin-1-yl)piperidin-1-yl)phenyl)piperazin-1-yl)(phenyl)methanone

In a manner similar to the synthesis of TRV 1144, (4-(3-(isopropylamino)-4-nitrophenyl)piperazin-1-yl)(phenyl)methanone 4-Bromo-2-fluoro-1-nitrobenzene (0.594 g, 2.70 mmol) was first reacted with 4-(1-pyrrolidinyl)piperidine (0.50 g, 3.24 mmol) to provide 0.41 g of an orange oil (43% yield). This material (0.41 g, 1.16 mmol) was dissolved in NMP (5 mL) and reacted in a similar manner as described in the synthesis of TRV 1144, (4-(3-(isopropylamino)-4-nitrophenyl)piperazin-1-yl)(phenyl)methanone with benzolpiperazine (0.439 g, 2.31 mmol) to afford the desired material in crude form. Purification with flash chromatography (5% MeOH/CH₂Cl₂) afforded 0.2028 g (38% yield) of TRV 1139, (4-(4-nitro-3-(4-(pyrrolidin-1-yl)piperidin-1-yl)phenyl)piperazin-1-yl)(phenyl)methanone. Further purification by recrystallizing from EtOAc/Hexane resulted in TRV 1139, (4-(4-nitro-3-(4-(pyrrolidin-1-yl)piperidin-1-yl)phenyl)piperazin-1-yl)(phenyl)methanone as orange crystals.

¹H NMR (500 MHz, DMSO) δ=7.91 (d, J=9.4 Hz, 1H), 7.50-7.44 (m, 5H), 6.57 (dd, J=9.4, 2.5 Hz, 1H), 6.41 (d, J=2.5 Hz, 1H), 3.74-3.47 (m, 8H), 3.23-3.21 (m, 2H), 2.81-2.77 (m, 2H), 2.54 (m, 4H), 2.16 (br s, 1H), 1.92-1.89 (m, 2H), 1.70 (m, 4H), 1.61-1.57 (m, 2H).

### Example 38

### Synthesis of TRV 1138

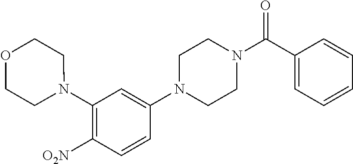 [SMILES: O=C(c1ccccc1)N1CCN(c2ccc([N+](=O)[O-])c(N3CCOCC3)c2)CC1]

### TRV 1138—(4-(3-morpholino-4-nitrophenyl)piperazin-1-yl)(phenyl)methanone

In a manner similar to the synthesis of TRV 1144, (4-(3-(isopropylamino)-4-nitrophenyl)piperazin-1-yl)(phenyl)methanone 4-Bromo-2-fluoro-1-nitrobenzene (1.00 g, 4.54 mmol) was first reacted with morpholine (0.59 mL, 6.81 mmol) to provide 1.27 g of an orange solid (97% yield). This material (0.88 g, 3.06 mmol) was dissolved in NMP (9 mL) and DIPEA (1.1 mL, 6.13 mmol) and reacted in a similar manner as described in the synthesis of TRV 1144, (4-(3-(isopropylamino)-4-nitrophenyl)piperazin-1-yl)(phenyl)methanone with benzolpiperazine hydrochloride (1.39 g, 6.13 mmol) to afford the desired material in crude form. Purification with flash chromatography (50% EtOAc/hexane) afforded 1.05 g (86% yield) of TRV 1138—(4-(3-morpholino-4-nitrophenyl)piperazin-1-yl)(phenyl)methanone. Further purification by recrystallization from EtOAc/Hexane resulted in 0.272 g of TRV 1138—(4-(3-morpholino-4-nitrophenyl)piperazin-1-yl)(phenyl)methanone as orange crystals. ¹H NMR (500 MHz, DMSO) 8=7.94 (d, J=9.5 Hz, 1H), 7.50-7.45 (m, 5H), 6.62 (dd, J=9.5, 2.5 Hz, 1H), 6.44 (d, J=2.5 Hz, 1H), 3.73 (t, J=4.5 Hz, 4H), 3.73-3.48 (m, 8H), 3.01 (t, J=4.5 Hz, 4H).

### Example 39

### Synthesis of TRV 1137

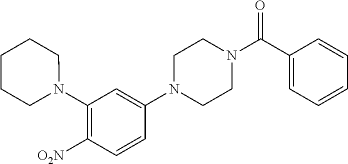 [SMILES: O=C(c1ccccc1)N1CCN(c2ccc([N+](=O)[O-])c(N3CCCCC3)c2)CC1]

### TRV 1137—(4-(4-nitro-3-(piperidin-1-yl)phenyl)piperazin-1-yl)(phenyl)methanone

In a manner similar to the synthesis of TRV 1144, (4-(3-(isopropylamino)-4-nitrophenyl)piperazin-1-yl)(phenyl)methanone 4-Bromo-2-fluoro-1-nitrobenzene (1.00 g, 4.54 mmol) was first reacted with piperidine (0.67 mL, 6.81 mmol) to provide 1.28 g of an orange solid (97% yield). This material (0.79 g, 2.77 mmol) was dissolved in NMP (8 mL) and DIPEA (1 mL, 5.54 mmol) and reacted in a similar manner as described in the synthesis of TRV 1144, (4-(3-(Isopropylamino)-4-nitrophenyl)piperazin-1-yl)(phenyl)methanone with benzolpiperazine hydrochloride (1.26 g, 5.54 mmol) to afford the desired material in crude form. Purification with flash chromatography (50% EtOAc/hexane) afforded 0.32 g (29% yield) of TRV 1137, (4-(4-nitro-3-(piperidin-1-yl)phenyl)piperazin-1-yl)(phenyl)methanone. Further purification by recrystallization from EtOAc/Hexane resulted in 0.190 g of TRV 1137, (4-(4-nitro-3-(piperidin-1-yl)phenyl)piperazin-1-yl)(phenyl)methanone as yellow needles.

¹H NMR (500 MHz, DMSO) δ=7.90 (d, J=9.4 Hz, 1H), 7.50-7.45 (m, 5H), 6.57 (dd, J=9.4, 2.5 Hz, 1H), 6.41 (d, J=2.5 Hz, 1H), 3.75-3.47 (m, 8H), 2.97-2.95 (m, 4H), 1.67-1.62 (m, 4H), 1.57-1.55 (m, 2H).

### Example 40

### Synthesis of TRV 1135

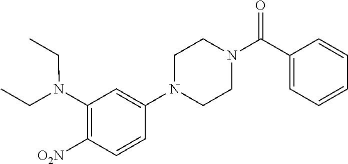 [SMILES: CCN(CC)c1cc(N2CCN(C(=O)c3ccccc3)CC2)ccc1[N+](=O)[O-]]

### TRV 1135—(4-(3-(diethylamino)-4-nitrophenyl)piperazin-1-yl)(phenyl)methanone

In a manner similar to the synthesis of TRV 1144, (4-(3-(isopropylamino)-4-nitrophenyl)piperazin-1-yl)(phenyl)methanone 4-Bromo-2-fluoro-1-nitrobenzene (1.00 g, 4.54 mmol) was reacted with diethylamine (0.71 mL, 6.81 mmol) to afford 1.20 g (97% yield) of orange oil. This material (0.94 g, 3.44 mmol) was dissolved in NMP (9.4 mL) and DIPEA (1.20 mL, 6.88 mmol) and reacted in a similar manner as described in the synthesis of TRV 1144, (4-(3-(Isopropylamino)-4-nitrophenyl)piperazin-1-yl)(phenyl)methanone with benzoylpiperazine hydrochloride (1.56 g, 6.88 mmol) to afford the desired material in crude form. Purification via flash column chromatography (45% EtOAc/Hex) provided 0.33 g of orange solid (25% yield) TRV 1135—(4-(3-(diethylamino)-4-nitrophenyl)piperazin-1-yl)(phenyl)methanone. ¹H NMR (500 MHz, CDCl3) δ=7.85 (d, J=9.1 Hz, 1H), 7.45-7.41 (m, 5H), 6.37 (d, 2.6 Hz, 1H), 6.35-6.34 (m, 1H), 3.91-3.24 (m, 8H), 3.17 (q, J=7.1 Hz, 4H), 1.11 (t, J=7.1 Hz, 6H).

### Example 41

### Synthesis of TRV 1098 and TRV 1099

Scheme for TRV 1098 and TRV 1099

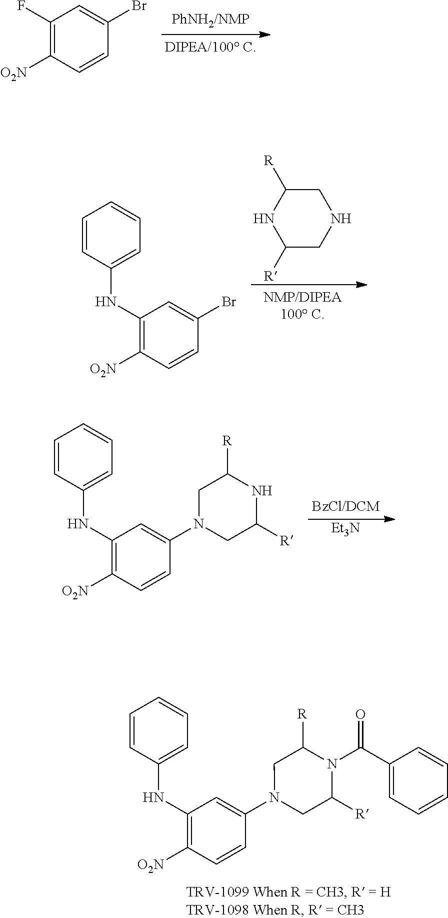

### Synthesis of (2,6-dimethyl-4-(4-nitro-3-(phenyl amino)phenyl)piperazin-1-yl)(phenyl)methanone (TRV 1098)

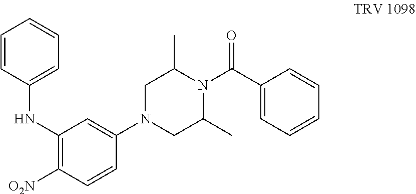 [SMILES: CC1CN(c2ccc([N+](=O)[O-])c(Nc3ccccc3)c2)CC(C)N1C(=O)c1ccccc1]

A solution of 5-bromo-2-nitro-N-phenylaniline (1.5 g, 5.0 mmol) and 2,6-dimethylpiperazine (0.885 g 7.5 mmol) in NMP (15 mL) was heated to 110° C. for 16 h. The solution whole cooled to rt to which was slowly added H₂O (5 mL). The resulting yellow precipitate was filtered, washed with 10 mL of water, dried (high vacuum, 14 h) to furnish 5-(3,5-dimethylpiperazin-1-yl)-2-nitro-N-phenylaniline (1.0 g), 65% yield. It was used in next step without further purification

Crude solution of 5-(3,5-dimethylpiperazin-1-yl)-2-nitro-N-phenylaniline (1.0 g, 3.06 mmol), benzoyl chloride (0.4 ml, 3.6 mmol) and triethylamine (1.27 ml, 9.18 mmol) in 20 ml DCM was stirred at 0° C. for 2 hours. The mixture was washed with water (2×10 ml) and dried over anhydrous sodium sulfate before removing the solvents. (2,6-dimethyl-4-(4-nitro-3-(phenylamino) phenyl)piperazin-1-yl)(Phenyl)methanone TRV 1098 (0.8 g, 68%) was obtained by flash column chromatograph (40% ethyl acetate in hexanes). ¹H NMR (500 MHz, CDCl₃): δ 1.28 (d, J=10.0 Hz, 3H), 1.36 (d, J=6.0 Hz, 3H), 3.22 (m, 2H), 3.58 (m, 2H), 4.55 (bs, 2H), 6.30 (dd, J=2.5, 9.5 Hz, 1H), 6.37 (d, J=2.5, Hz, 1H), 7.23 (m, 1H), 7.32 (m, 3H), 7.39 (m, 2H), 7.45 (m, 5H), 8.18 (d, J=10.0 Hz, 1H), 9.88 (s, 1H). HRMS (+ESI) calculated for C₂H₂N₄NaO₃: 453.1897. Found: 453.1893

### Synthesis of (2-methyl-4-(4-nitro-3-(phenylamino)phenyl)piperazin-1-yl)(phenyl)methanone (TRV 1099)

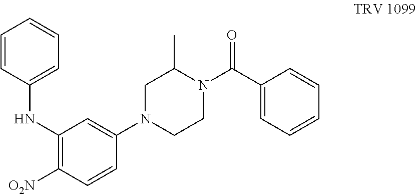 [SMILES: CC1CN(c2ccc([N+](=O)[O-])c(Nc3ccccc3)c2)CCN1C(=O)c1ccccc1]

TRV 1099 was synthesized following similar procedure as described for TRV 1098. ¹H NMR (500 MHz, CDCl₃): δ1.32 (d, J=6.5 Hz, 3H), 2.99 (m, 1H), 3.02 (m, 2H), 3.58 (m, 4H), 4.55 (bs, 2H), 6.30 (dd, J=2.5, 7.5 Hz, 1H), 6.37 (d, J=2.5, Hz, 1H), 7.25-7.47 (m, 10H), 8.17 (d, J=9.5 Hz, 1H), 9.87 (s, 1H). HRMS (+ESI) calculated for C₂H₂₄N₄NaO₃: 439.1741. Found: 439.1741

### Example 42

### Synthesis of 3-substituted amide analogs

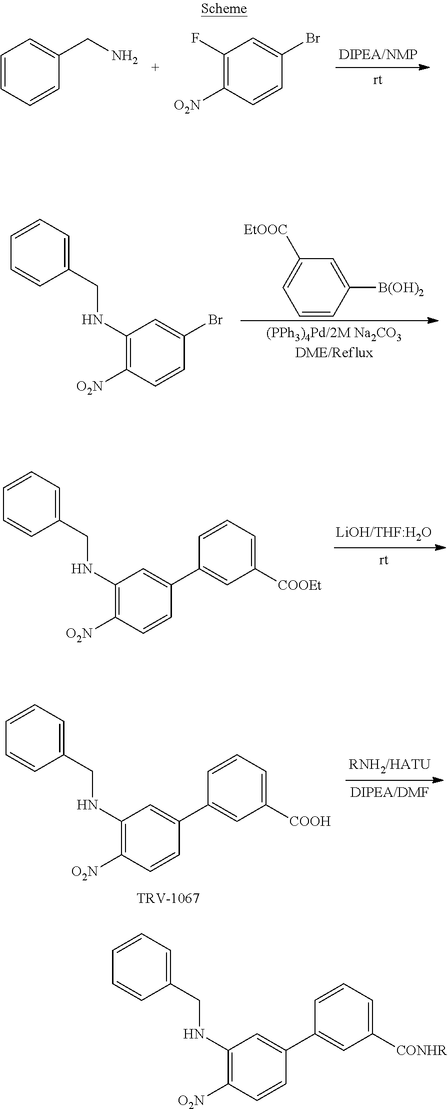

General Procedure for Amide Coupling Using HATU

To a solution of 3′-(benzylamino)-4′-nitrophenyl-3-carboxylic acid (TRV 1067) (1.0 mmol) and appropriate amine (11.0 mmol) in DMF (5 mL) was added diisopropylethylamine (1.5 mmol) under argon. The mixture cooled to 0° C., HATU (11.0 mmol) was added at that temperature and then stirred at room temperature for 4 h. After completion of reaction (by TLC), H₂O (5 mL) was added drop by drop. The resulting yellow precipitate was filtered, washed with 2 mL of water, dried (high vacuum, 14 h) to furnish corresponding carboxamide product. The following compounds of the Example were synthesized using this method.

### Synthesis of 3′-(benzyl amino)-N-methyl-4′-nitrobiphenyl-3-carboxamide (TRV 1101)

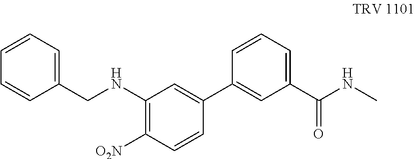 [SMILES: CNC(=O)c1cccc(-c2ccc([N+](=O)[O-])c(NCc3ccccc3)c2)c1]

¹H NMR (500 MHz, DMSO): δ 2.82 (d, J=4.5 Hz, 3H), 4.77 (d, J=6.0 Hz, 2H), 7.05 (dd, J=2.0, 9.0 Hz, 1H), 7.19 (d, J=9.0 Hz, 1H), 7.27 (t, J=7.0, 7.5 Hz, 1H), 7.35 (m, 2H), 7.45 (m, 2H), 7.57 (t, J=9.0, 7.5 Hz, 1H), 7.72 (dd, J=1.0, 1.5 Hz, 1H), 7.74 (dd, J=8.5, 1.6 Hz, 1H), 7.86 (s, 1H), 7.88 (dd, J=1.5, 5.0 Hz, 1H), 8.65 (m, 1H), 8.80 (m, 1H). HRMS (+ESI) calculated for C₂₁H₁₉N₃NaO₃: 384.1319. Found: 384.1314

### Synthesis of 3′-(benzyl amino)-N,N-dimethyl-4′-nitrobiphenyl-3-carboxamide (TRV 1102)

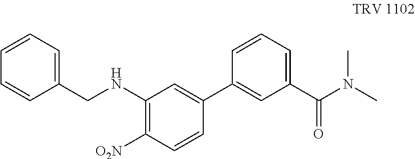 [SMILES: CN(C)C(=O)c1cccc(-c2ccc([N+](=O)[O-])c(NCc3ccccc3)c2)c1]

¹H NMR (500 MHz, DMSO): δ 2.89 (s, 3H), 3.02 (s, 3H), 4.78 (d, J=5.5 Hz, 2H), 7.02 (dd, J=2.0, 9.0 Hz, 1H), 7.13 (d, J=1.5 Hz, 1H), 7.27 (m, 1H), 7.35 (m, 2H), 7.45 (m, 3H), 7.57 (m, 2H), 7.68 (m, 1H), 8.16 (d, J=9.0 Hz, 1H), 8.78 (m, 1H). HRMS (+ESI) calculated for C₂₂H₂₁N₃NaO₃: 398.1475. Found: 398.1470.

### Synthesis of 3′-(benzyl amino)-4′-nitrobiphenyl-3-carboxamide (TRV 1103)

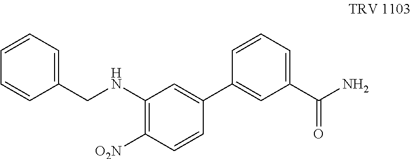 [SMILES: NC(=O)c1cccc(-c2ccc([N+](=O)[O-])c(NCc3ccccc3)c2)c1]

¹H NMR (500 MHz, DMSO): δ 4.77 (d, J=6.0 Hz, 2H), 7.05 (dd, J=2.0, 9.0 Hz, 1H), 7.20 (d, J=9.0 Hz, 1H), 7.27 (t, J=7.0, 7.5 Hz, 1H), 7.35 (m, 2H), 7.45 (m, 2H), 7.57 (t, J=9.0, 7.5 Hz, 1H), 7.72 (dd, J=1.0, 1.5 Hz, 1H), 7.74 (dd, J=8.5, 1.6 Hz, 1H), 7.86 (s, 1H), 7.88 (dd, J=1.5, 5.0 Hz, 1H), 8.09 (m, 2H), 8.11 (d, J=10.5 Hz, 1H), 8.75 (m, 1H). HRMS (+ESI) calculated for C₂₀H₁₇N₃NaO₃: 370.1162. Found: 370.1129.

### Synthesis of 3′-(benzyl amino)-N,N-diethyl-4′-nitrobiphenyl-3-carboxamide (TRV 1110)

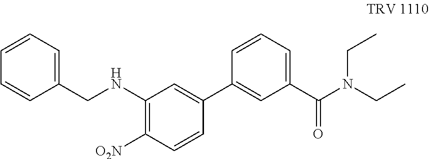 [SMILES: CCN(CC)C(=O)c1cccc(-c2ccc([N+](=O)[O-])c(NCc3ccccc3)c2)c1]

¹H NMR (500 MHz, DMSO): δ 1.08 (m, 3H), 1.18 (m, 3H), 3.16 (m, 2H), 3.46 (m, 2H), 4.77 (d, J=5.5 Hz, 2H), 7.01 (dd, J=1.5, 9.0 Hz, 1H), 7.11 (s, 1H), 7.24 (m, 1H), 7.37 (m, 6H), 7.54 (m, 1H), 7.65 (m, 1H), 8.16 (d, J=9.0 Hz, 1H), 8.80 (t, J=5.5, 6.0 Hz, 1H). HRMS (+ESI) calculated for C₂H₂₅N₃NaO₃: 426.1788. Found: 426.1771.

### Synthesis of (3′-(benzyl amino)-4′-nitrobiphenyl-3-yl)(piperidin-1-yl)methanone (TRV 1111)

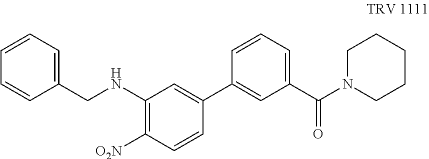 [SMILES: O=C(c1cccc(-c2ccc([N+](=O)[O-])c(NCc3ccccc3)c2)c1)N1CCCCC1]

¹H NMR (500 MHz, CDCl₃): δ 1.52 (bs, 2H), 1.72 (bs, 4H), 3.36 (bs, 2H), 3.77 (bs, 2H), 4.65 ((d, J=5.5 Hz, 2H), 6.91 (dd, J=1.5, 9.0 Hz, 1H), 7.01 (s, 1H), 7.35-7.55 (m, 9H), 8.30 (d, J=9.0 Hz, 1H), 8.60 (m, 1H). HRMS (+ESI) calculated for C₂₅H₂₅N₃NaO₃: 438.1788. Found: 438.1757.

### Synthesis of 3′-(benzyl amino)-N,N-diisopropyl-4′-nitrobiphenyl-3-carboxamide (TRV 1112)

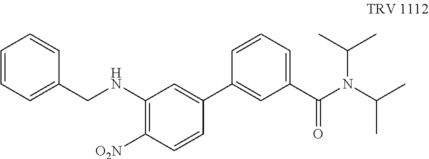 [SMILES: CC(C)N(C(=O)c1cccc(-c2ccc([N+](=O)[O-])c(NCc3ccccc3)c2)c1)C(C)C]

¹H NMR (500 MHz, CDCl₃): δ 1.21 (bs, 6H), 1.63 (bs, 6H), 3.49 (bs, 1H), 3.53 (bs, 1H), 4.66 ((d, J=5.5 Hz, 2H), 6.91 (dd, J=1.0, 9.0 Hz, 1H), 7.01 (s, 1H), 7.35-7.52 (m, 9H), 8.31 (d, J=9.0 Hz, 1H), 8.57 (m, 1H). HRMS (+ESI) calculated for C₂₆H₂₉N₃NaO₃: 454.2101. Found: 454.2078.

### Synthesis of (3′-(benzyl amino)-4′-nitrobiphenyl-3-yl)(morpholino)methanone (TRV 1113)

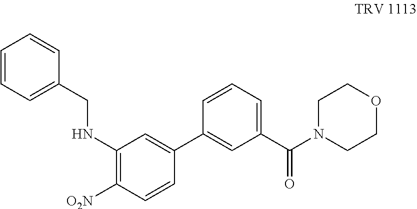 [SMILES: O=C(c1cccc(-c2ccc([N+](=O)[O-])c(NCc3ccccc3)c2)c1)N1CCOCC1]

¹H NMR (500 MHz, CDCl₃): δ 3.31 (bs, 2H), 3.52 (bs, 2H), 3.66 (bs, 4H), 4.77 (d, J=6.0 Hz, 2H), 7.05 (dd, J=2.0, 9.0 Hz, 1H), 7.13 (d, J=2.0 Hz, 1H), 7.27 (m, 1H), 7.35-7.55 (m, 8H), 8.18 (d, J=9.0 Hz, 1H), 8.78 (t, J=5.5, 6.0 Hz, 1H). HRMS (+ESI) calculated for C₂₄H₂₃N₃NaO₃: 440.1581. Found: 440.1550.

### Synthesis of 1-(4-(3′-(benzyl amino)-4′-nitrobiphenylcarbonyl)piperazin-1-yl)ethanone (TRV 1114)

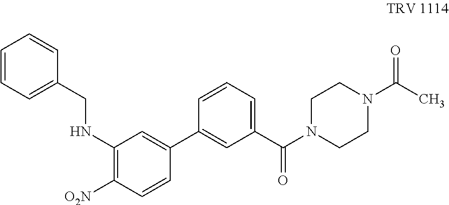 [SMILES: CC(=O)N1CCN(C(=O)c2cccc(-c3ccc([N+](=O)[O-])c(NCc4ccccc4)c3)c2)CC1]

¹H NMR (500 MHz, DMSO): δ2.06 (s, 3H), 3.28-3.42 (m, 4H), 3.55-3.68 (m, 4H), 4.77 (d, J=6.0 Hz, 2H), 7.03 (dd, J=2.0, 9.0 Hz, 1H), 7.13 (s, 1H), 7.27 (m, 1H), 7.34-7.71 (m, 8H), 8.19 (d, J=8.5 Hz, 1H), 8.79 (t, J=6.0 Hz, 1H). HRMS (+ESI) calculated for C₂₆H₂₆N₄NaO₄: 48.1.1846. Found: 481.1800.

### Synthesis of (3′-(benzyl amino)-4′-nitrobiphenyl-3-yl)(4-methylpiperazin-1-yl)methanone (TRV 1115)

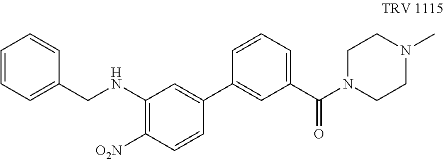 [SMILES: CN1CCN(C(=O)c2cccc(-c3ccc([N+](=O)[O-])c(NCc4ccccc4)c3)c2)CC1]

¹H NMR (500 MHz, DMSO): δ 2.20 (s, 3H), 2.23 (bs, 2H), 2.38 (bs, 2H), 3.29 (bs, 2H), 3.65 (m, 2H), 4.78 (d, J=6.0 Hz, 2H), 7.01 (dd, J=2.0, 9.0 Hz, 1H), 7.13 (d, J=2.0 Hz, 1H), 7.26 (m, 1H), 7.34-7.69 (m, 8H), 8.18 (d, J=8.5 Hz, 1H), 8.77 (m, 1H). HRMS (+ESI) calculated for C₂₅H₂₇N₄O₃: 431.2078. Found: 431.2059.

### Synthesis of 3′-(benzyl amino)-N-ethyl-4′-nitrobiphenyl-3-carboxamide (TRV 1116)

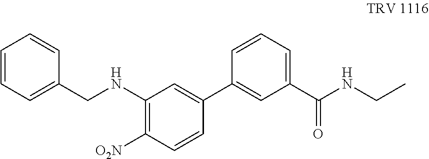 [SMILES: CCNC(=O)c1cccc(-c2ccc([N+](=O)[O-])c(NCc3ccccc3)c2)c1]

¹H NMR (500 MHz, DMSO): δ 1.15 (t, J=7.0, 7.5 Hz, 3H), 3.31 (q, J=1.5, 7.0 Hz, 2H), 4.77 (d, J=6.0 Hz, 2H), 7.05 (dd, J=1.5, 9.0 Hz, 1H), 7.19 (d, J=1.5 Hz, 1H), 7.26 (m, 1H), 7.38-7.57 (m, 5H), 7.73 (d, J=8.0 Hz, 1H), 7.88 (d, J=7.5 Hz, 1H), 8.04 (s, 1H), 8.21 (d, J=8.5 Hz, 1H), 8.58 (m, 1H) 8.82 (m, 1H). HRMS (+ESI) calculated for C₂₂H₂₁N₃NaO₃: 398.1475. Found: 398.1452.

### Synthesis of (3′-(benzylamino)-4′-nitrobiphenyl-3-yl)(pyrrolidin-1-yl)methanone (TRV 1117)

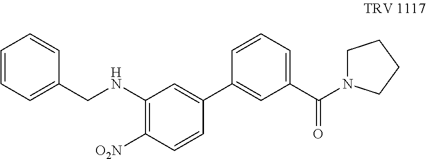 [SMILES: O=C(c1cccc(-c2ccc([N+](=O)[O-])c(NCc3ccccc3)c2)c1)N1CCCC1]

¹H NMR (500 MHz, DMSO): δ 1.83 (m, 2H), 1.88 (m, 2H), 3.32 (m, 2H), 3.49 (m, 2H), 4.78 (d, J=6.0 Hz, 2H), 7.00 (dd, J=2.0, 7.0 Hz, 1H), 7.03 (s, 1H), 7.12-7.71 (m, 9H), 8.18 (d, J=9.0 Hz, 1H), 8.80 (t, J=6.0 Hz, 1H). HRMS (+ESI) calculated for C₂₄H₂₃N₃NaO₃: 424.1632. Found: 424.1616.

### Synthesis of N-benzyl-3′-(benzylamino)-N-methyl-4′-nitrobiphenyl-3-carboxamide (TRV 1118)

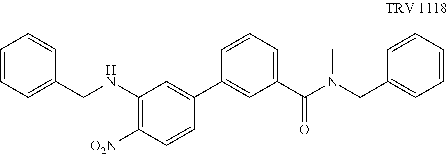 [SMILES: CN(Cc1ccccc1)C(=O)c1cccc(-c2ccc([N+](=O)[O-])c(NCc3ccccc3)c2)c1]

¹H NMR (500 MHz, DMSO): δ 2.82 (s, 3H), 4.48 (s, 1H), 4.76 (m, 3H), 6.86-7.70 (m, 16H), 8.16 (dd, J=8.5, 9.0 Hz, 1H), 8.80 (m, 1H). HRMS (+ESI) calculated for C₂₈H₂₅N₃NaO₃: 474.1788. Found: 474.1771.

### Synthesis of 3′-(benzyl amino)-4′-nitro-N,N-dipropylbiphenyl-3-carboxamide (TRV 1130)

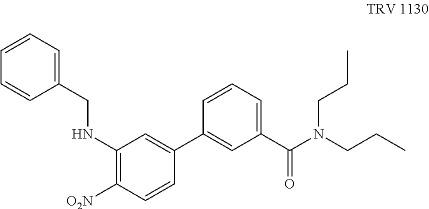 [SMILES: CCCN(CCC)C(=O)c1cccc(-c2ccc([N+](=O)[O-])c(NCc3ccccc3)c2)c1]

¹H NMR (500 MHz, DMSO): δ 0.77 (m, 3H), 1.04 (m, 3H), 1.29 (m, 2H), 1.62 (m, 2H) 3.18 (m, 2H), 3.52 (m, 2H), 4.65 (d, J=5.5 Hz, 2H), 6.92 (dd, J=1.5, 9.0 Hz, 1H), 7.01 (d, J=1.5 Hz1H), 7.34-7.53 (m, 9H), 8.31 (d, J=9.0 Hz, 1H), 8.56 (t, J=5.0, 5.5, Hz, 1H).

### Synthesis of (1-(3-(benzylamino)-4-nitrophenyl)piperidin-3-yl)(pyrrolidin-1-yl)methanone (TRV 1131)

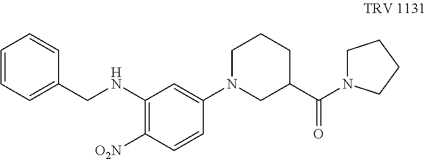 [SMILES: O=C(C1CCCN(c2ccc([N+](=O)[O-])c(NCc3ccccc3)c2)C1)N1CCCC1]

¹H NMR (500 MHz, DMSO): δ1.38 (m, 1H), 1.61 (m, 2H), 1.74-1.90 (m, 5H), 2.45 (m, 1H), 2.88-2.97 (m, 2H), 3.23-3.40 (m, 4H), 3.92 (m, 2H), 4.58 (d, J=5.5 Hz, 2H), 5.93 (d, J=2.5 Hz, 1H), 6.43 (dd, J=2.5, 10.0 Hz, 1H), 7.26 (m, 1H), 7.33-7.39 (m, 4H), 7.90 (d, J=10.0 Hz, 1H), 8.84 (t, J=5.5, 6.0 Hz, 1H).

### Synthesis of 1-(3-(benzyl amino)-4-nitrophenyl)-N,N-dimethylpiperidine-3-carboxamide (TRV 1132)

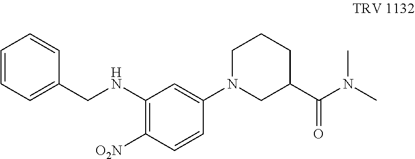 [SMILES: CN(C)C(=O)C1CCCN(c2ccc([N+](=O)[O-])c(NCc3ccccc3)c2)C1]

¹H NMR (500 MHz, DMSO): δ1.42 (m, 2H), 10.60 (m, 2H), 2.82 (s, 3H), 2.86-3.00 (m, 3H), 3.05 (s, 3H), 3.87 (m, 2H), 4.58 (d, J=6.0 Hz, 2H), 5.91 (d, J=2.0 Hz, 1H), 6.4 (dd, J=2.5, 10.0 Hz, 1H), 7.27 (m, 1H), 7.32-7.39 (m, 4H), 7.90 (d, J=9.5 Hz, 1H), 8.84 (t, J=4.0, 5.5 Hz, 1H).

### Synthesis of (1-(3-(benzyl amino)-4-nitrophenyl)piperidin-4-yl)(pyrrolidin-1-yl)methanone (TRV 1133)

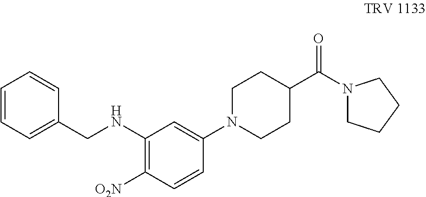 [SMILES: O=C(C1CCN(c2ccc([N+](=O)[O-])c(NCc3ccccc3)c2)CC1)N1CCCC1]

¹H NMR (500 MHz, DMSO): δ1.44 (m, 2H), 1.67 (m, 2H), 1.78 (m, 2H), 1.88 (m, 2H), 2.73 (m, 1H), 2.97 (m, 2H), 3.25 (m, 2H), 3.48 (m, 2H), 3.91 (m, 2H), 4.58 (d, J=5.5 Hz, 2H), 5.94 (d, J=2.0 Hz, 1H), 6.41 (dd, J=2.5, 9.5 Hz, 1H), 7.27 (m, 1H), 7.34-7.41 (m, 4H), 7.90 (d, J=9.5 Hz, 11H), 8.84 (t, J=5.5, 6.0 Hz, 1H).

### Synthesis of 1-(3-(benzyl amino)-4-nitrophenyl)-N,N-dimethylpiperidine-4-carboxamide (TRV 1134)

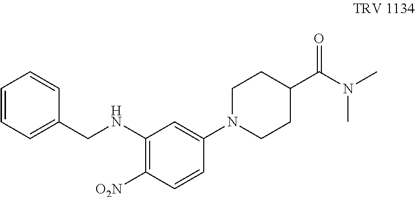 [SMILES: CN(C)C(=O)C1CCN(c2ccc([N+](=O)[O-])c(NCc3ccccc3)c2)CC1]

¹H NMR (500 MHz, DMSO): δ 1.45 (m, 2H), 1.64 (m, 2H), 2.80 (s, 3H), 2.89-3.00 (m, 3H), 3.03 (s, 3H), 3.91 (m, 2H), 4.59 (d, J=6.0 Hz, 2H), 5.93 (d, J=2.0 Hz, 1H), 6.39 (dd, J=2.5, 10.0 Hz, 1H), 7.27 (m, 1H), 7.34-7.41 (m, 4H), 7.91 (d, J=9.5 Hz, 1H), 8.83 (t, J=4.0, 5.5 Hz, 1H).

### Example 43

### Synthesis of TRV 1128

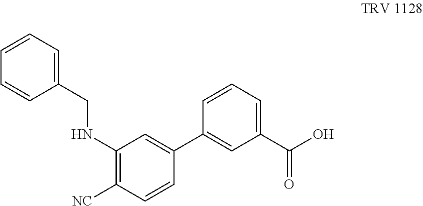 [SMILES: N#Cc1ccc(-c2cccc(C(=O)O)c2)cc1NCc1ccccc1]

Scheme for TRV 1128

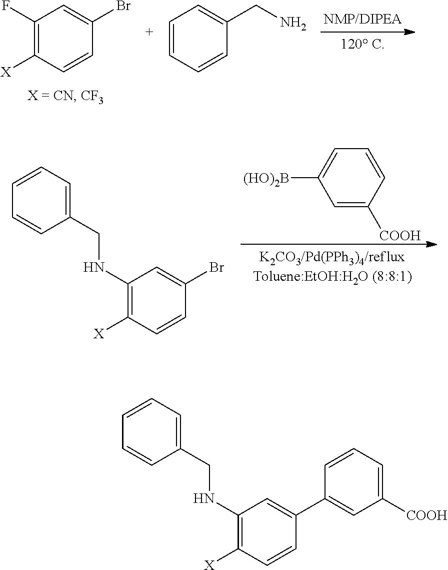

### Synthesis of 3′-(benzyl amino)-4′-cyanobiphenyl-3-carboxylic acid (TRV 1128)

To a solution of 2-(benzyl amino)-4-bromobenzonitrile (0.572 g 2.0 mmol) and 3-carboxyphenylboronic acid (0.396 g, 2.4 mmol) in Toluene: EtOH: H₂O (8:8:1) (17 mL) was added K₂CO₃ (0.828 g 6 mmol). After stirring reaction for 15 min under argon tetrakis(triphenylphosphine) palladium (120 mg, 0.1 mol) was added, resulting mixture was then heated at 100° C. for 12 h. The reaction mixture was cooled to room temperature and diluted with ethyl acetate and extracted three times with water. The aqueous layer was then acidified with 2M HCl to get 3′-(benzyl amino)-4′-cyanobiphenyl-3-carboxylic acid TRV 1128 (0.49 g 75%).

¹H NMR (500 MHz, DMSO): δ4.55 (d, J=6.0 Hz, 2H), 6.88 (d, J=1.5 Hz, 1H), 6.93 (dd, J=1.5, 8.0, 1 H), 7.02 (t, J=6.0, 6.5 Hz, 1H), 7.22 (m, 1H), 7.35 (m, 2H), 7.42 (m, 2H), 7.58 (m, 2H), 7.75 (m, 1H), 7.87 (s, 1H), 8.04 (s, 1H). 13.15 (bs, 11H)

### Example 44

### Synthesis of TRV 1124 and TRV 1129

Scheme for TRV 1124 and TRV 1129

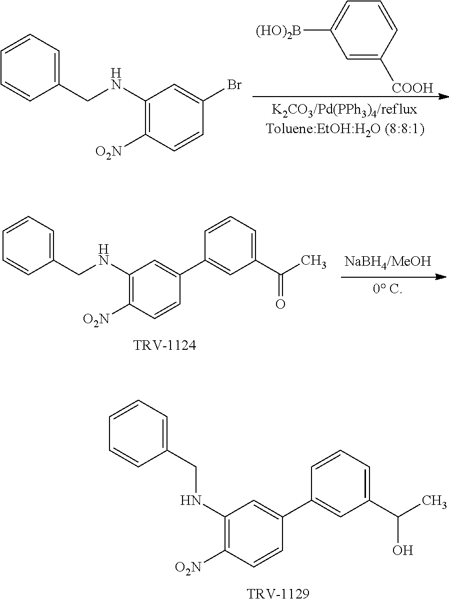 [SMILES: O=[N+]([O-])c1ccc(Br)cc1NCc1ccccc1 ; O=C(O)c1cccc(B(O)O)c1 ; CC(=O)c1cccc(-c2ccc([N+](=O)[O-])c(NCc3ccccc3)c2)c1 ; CC(O)c1cccc(-c2ccc([N+](=O)[O-])c(NCc3ccccc3)c2)c1]

### Synthesis of 1-(3′-(benzyl amino)-4′-nitrobiphenyl-3-yl)ethanone (TRV 1124)

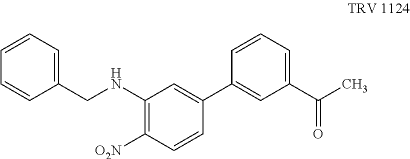 [SMILES: CC(=O)c1cccc(-c2ccc([N+](=O)[O-])c(NCc3ccccc3)c2)c1]

To a solution of N-benzyl-5-bromo-2-nitrobenzenamine (0.306 g, 1.0 mmol) and 3-acetylphenylboronic acid (0.195 g, 1.2 mmol) in Toluene: EtOH: H₂O (8:8:1) (17 mL) was added K₂CO₃ (0.414 g, 3 mmol). After stirring reaction for 15 min under argon tetrakis(triphenylphosphine) palladium (60 mg, 0.05 mol) was added, resulting mixture was then heated at 100° C. for 12 h. The reaction mixture was cooled to room temperature and diluted with ethyl acetate and extracted three times with water. The aqueous layer was then acidified with 2M HCl to get 1-(3′-(benzyl amino)-4′-nitrobiphenyl-3-yl)ethanone TRV 1124 (0.250 g, 72%).

¹H NMR (500 MHz, DMSO): δ 2.63 (s, 3H), 4.77 (d, J=6.0 Hz, 2H), 7.05 (dd, J=1.5, 2.0, 8.5, 9.0 Hz, 1H), 7.18 (d, J=1.5 Hz, 1H), 7.27 (m, 1H), 7.37 (m, 2H), 7.47 (m, 2H), 7.63 (t, J=7.5, 8.0 Hz, 1H), 7.86 (dd, J=0.5, 1.5, 5.5 Hz, 1H), 7.99 (dd, J=2.0, 8.0 Hz, 1H), 8.05 (dd, J=1.5, 8.0 Hz, 1H), 8.20 (d, J=8.5 Hz, 1H), 8.81 (t, J=6.0 Hz, 1H).

### Synthesis of 1-(3′-(benzyl amino)-4′-nitrobiphenyl-3-yl)ethanol (TRV 1129)

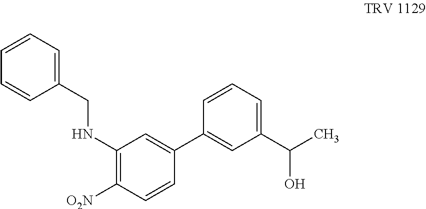 [SMILES: CC(O)c1cccc(-c2ccc([N+](=O)[O-])c(NCc3ccccc3)c2)c1]

To a stirred solution of 1-(3′-(benzyl amino)-4′-nitrobiphenyl-3-yl)ethanone TRV 1124 (0.250 g, 0.72 mmol) in MeOH (5 mL) was added NaBH₄ (0.035 g, 0.864 mmol) at 0° C. After completion of reaction by TLC, it was quenched with H₂O and extracted twice with EtOAc. The combined organic layer was evaporated under vacuum to get the crude product. Column chromatography on silica gel gave 1-(3′-(benzyl amino)-4′-nitrobiphenyl-3-yl)ethanol TRV 1129 (0.188 g, 75%).

¹H NMR (500 MHz, DMSO): δ1.56 (d, J=6.5 Hz, 3H), 1.90 (m, 1H) 4.66 (d, J=5.5 Hz, 2H), 4.98 (m, 1H), 6.93 (dd, J=1.5, 2.0, 9.0 Hz, 1H), 7.02 (d, J=1.5 Hz, 1H), 7.35 (m, 1H), 7.42 (m, 7H), 7.49 (s, 1H), 8.28 (d, J=8.5 Hz, 1H), 8.58 (m, 1H).

### Example 45

### Synthesis of TRV 1155

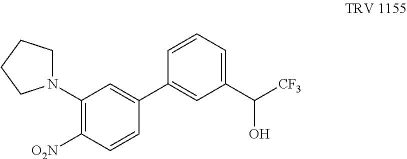 [SMILES: O=[N+]([O-])c1ccc(-c2cccc(C(O)C(F)(F)F)c2)cc1N1CCCC1]

Scheme for TRV 1155

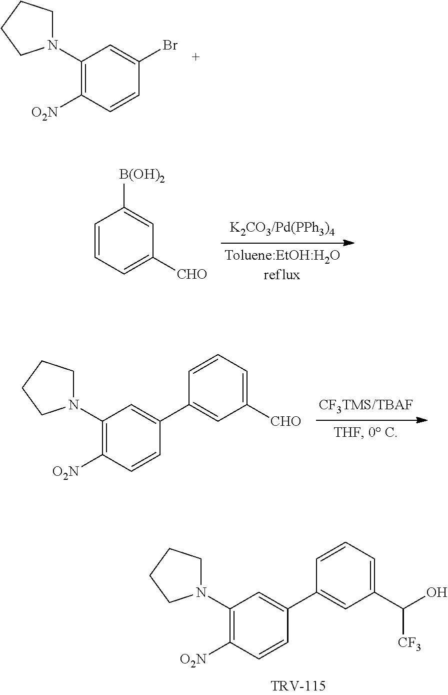 [SMILES: O=[N+]([O-])c1ccc(Br)cc1N1CCCC1 ; O=Cc1cccc(B(O)O)c1 ; O=Cc1cccc(-c2ccc([N+](=O)[O-])c(N3CCCC3)c2)c1 ; O=[N+]([O-])c1ccc(-c2cccc(C(O)C(F)(F)F)c2)cc1N1CCCC1]

### Synthesis of 2,2,2-trifluoro-1-(4′-nitro-3′-(pyrrolidin-1-yl)biphenyl-3-yl)ethanol (TRV 1155)

A mixture of 4′-nitro-3′-(pyrrolidin-1-yl)biphenyl-3-carbaldehyde (0.5 g 1.68 mmol) and TMSCF₃ (0.287 g 2.02 mmol) in 10 mL of THF cooled to 0° C. was treated with a catalytic amount (ca. 20 mg) of TBAF. Instantaneously, a yellow color developed with the initial evolution of fluorotrimethylsilane, and the reaction mixture was brought to ambient temperature and stirred. The mixture was periodically analyzed by TLC for the completion of the reaction. The resulting siloxy compounds were then hydrolyzed with aqueous HCl. After the reaction, the mixture was extracted with ether (75 mL), and the ether extracts were washed with water (50 mL) and brine (50 mL), dried over Na₂SO₄, and concentrated. The residue was purified by flash column chromatography to give (0.38 g 62%) of 2,2,2-trifluoro-1-(4′-nitro-3′-(pyrrolidin-1-yl) biphenyl-3-yl)ethanol TRV 1155. ¹ NMR (500 MHz, DMSO): δ1.94 (m, 4H), 3.23 (m, 4H), 5.29 (m, 1H), 7.02 (dd, J=1.5, 8.5 Hz, 1H), 7.20 (d, J=1.5 Hz, 1H), 7.56 (m, 2H), 7.75 (m, 1H), 7.83 (m, 2H).

### Example 46

### Synthesis of TRV 1157

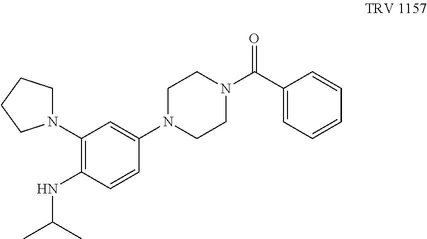 [SMILES: CC(C)Nc1ccc(N2CCN(C(=O)c3ccccc3)CC2)cc1N1CCCC1]

Scheme for TRV 1157

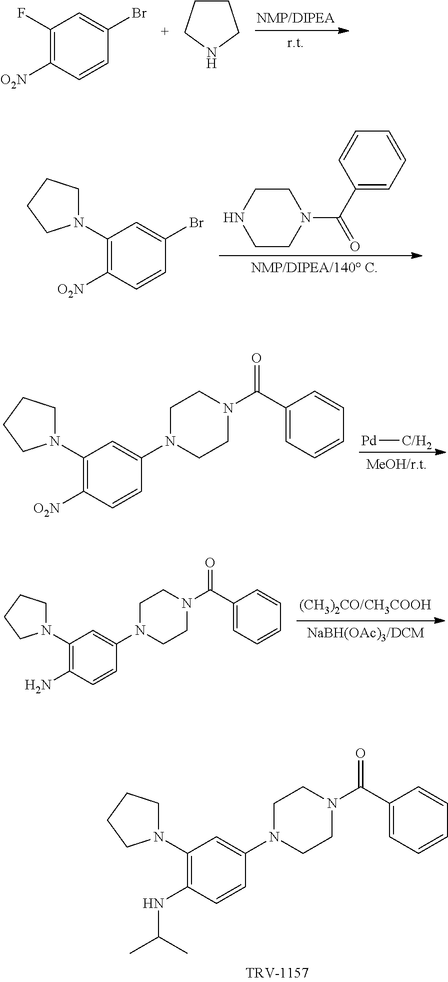

### Synthesis of (4-(4-(isopropyl amino)-3-(pyrrolidin-1-yl)phenyl)piperazin-1-yl)(phenyl)methanone (TRV 1157)

To a stirred solution of Acetone (0.04 mL, 0.035 g, 0.6 mmol) and (4-(4-amino-3-(pyrrolidin-1-yl)phenyl)piperazin-1-yl)(phenyl)methanone (0.175 g, 0.5 mmol) were mixed in DCM (5 mL) at rt under N₂. Sodium triacetoxyborohydride (0.158 g, 0.75 mmol) and glacial AcOH (0.045 g, 0.75 mmol) were added, and the mixture was stirred at room temperature for 12 h. The reaction mixture was quenched with aqueous saturated NaHCO₃ solution, and the product was extracted with Et₂O. The Et₂O extract was dried (MgSO₄) and concentrated under vacuum. The resultant residue was then purified by column chromatography to afford TRV 1157 as white solid (0.170 g, 70%). ¹H NMR (CDCl₃, 500 MHz): δ 1.32 (d, J=6.0 Hz, 6H). 1.84 (m, 4H), 1.97 (m, 4H), 3.04 (m, 6H), 3.15 (bs, 2H), 3.45 (m, 3H), 3.98 (m, 3H), 6.32-6.35 (m, 2H), 6.79 (d, J=2.5 Hz, 1H), 7.45-7.50 (m, 5H).

### Example 47

### Synthesis of TRV 1192

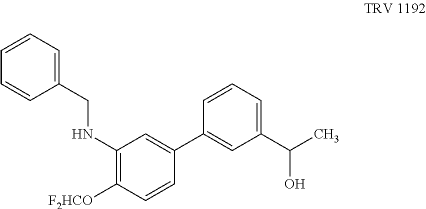 [SMILES: CC(O)c1cccc(-c2ccc(OC(F)F)c(NCc3ccccc3)c2)c1]

Scheme for TRV 1192

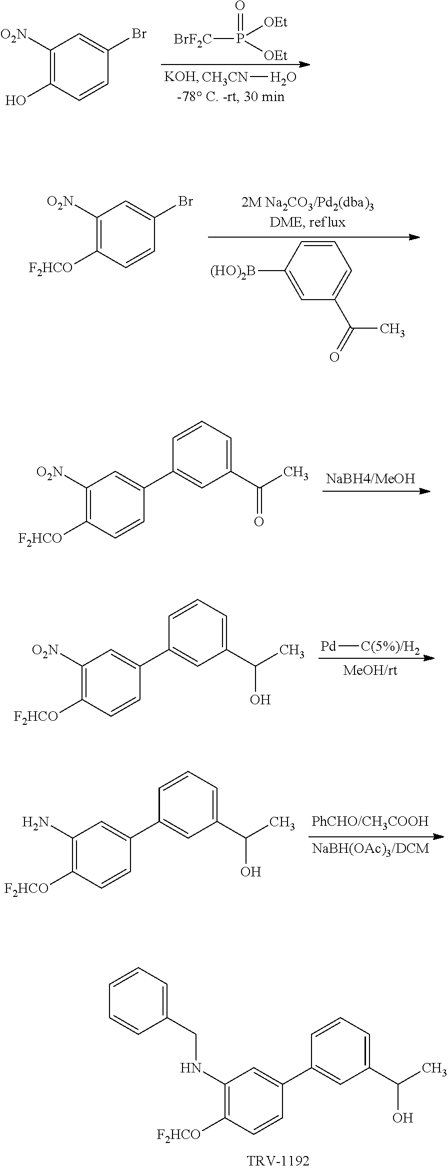

### Synthesis of 1-(3′-(benzyl amino)-4′-(difluoromethoxy)biphenyl-3-yl)ethanol (TRV 1192)

To a stirred solution of benzaldeyde (0.227 g, 2.15 mmol) and 1-(3′-amino-4′-(difluoromethoxy) biphenyl-3-yl)ethanol (0.5 g, 1.79 mmol) were mixed in DCM (5 mL) at rt under N₂. Sodium triacetoxyborohydride (0.56 g, 2.68 mmol) and glacial AcOH (0.160 g, 2.68 mmol) were added, and the mixture was stirred at rt for 12 h. The reaction mixture was quenched with aqueous saturated NaHCO₃ solution, and the product was extracted with Et₂O. The Et₂O extract was dried (MgSO₄) and concentrated under vacuum. The resultant residue was then purified by column chromatography to afford 1-(3′-(benzyl amino)-4′-(difluoromethoxy)biphenyl-3-yl)ethanol TRV 1192 as white solid (0.46 g, 69%). ¹HNMR (CDCl₃, 500 MHz): δ1.56 (d, J=6.5 Hz, 3H), 1.85 (s, 1H), 4.48 (d, J=4.0 Hz, 2H), 4.71 (m, 1H), 4.98 (m, 1H), 6.40-6.70 (t, J=74.5 Hz, 1H), 6.89 (m, 2H), 7.12 (d, J=4.0 Hz, 1H), 7.32-7.49 (m, 9H).

### Example 48

### Synthesis of TRV 1136

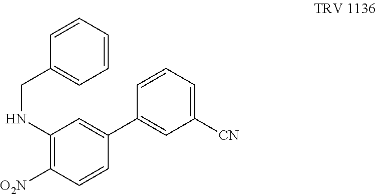 [SMILES: N#Cc1cccc(-c2ccc([N+](=O)[O-])c(NCc3ccccc3)c2)c1]

### Synthesis of 3′-(benzyl amino)-4′-nitrobiphenyl-3-carbonitrile (TRV 1136)

To a solution of N-benzyl-5-bromo-2-nitrobenzenamine (2.0 g, 6.5 mmol) and 3-cyanophenylboronic acid (1.23 g, 8.45 mmol) in DME (20 mL) was added 2M Na₂CO₃ solution (6.4 ml, 13.0 mmol). After stirring reaction for 15 min under argon tetrakis(triphenylphosphine) palladium (0.369 mg, 0.32 mol) was added, resulting mixture was then heated at 80° C. for 20 h. It was then filtered through celite, evaporated under vacuum to get crude product. Flash column chromatography gave 3′-(benzyl amino)-4′-nitrobiphenyl-3-carbonitrile TRV 1136 (1.6 g, 76%). ¹H NMR (500 MHz, CDCl₃): δ 4.77 (d, J=6.0 Hz, 2H), 7.04 (dd, J=2.0, 9.0 Hz, 1H), 7.22 (d, J=2.0 Hz, 1H), 7.27 (m, 1H), 7.36 (m, 2H), 7.45 (m, 2H), 7.68 (m, 1H), 7.85 (m, 1H), 7.96 (m, 1H), 8.10 (m, 1H), 8.18 (m, 2H), 8.78 (t, J=6.0 Hz, 1H).

### Example 49

### Synthesis of TRV 1120

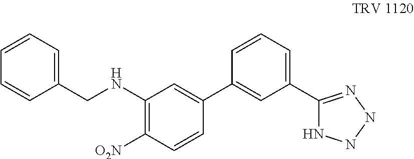 [SMILES: O=[N+]([O-])c1ccc(-c2cccc(C3=NNNN3)c2)cc1NCc1ccccc1]

A solution of TRV 1136 (250 mg, 0.76 mmol), sodium azide (59 mg, 0.91 mmol) and zinc bromide (503 mg, 2.28 mmol) in NMP was heated to 140° C. for 16 h. The solution was cooled, diluted with HCl (1M, 20 mL) and water (10 mL). The mixture was extracted with ethyl acetate (3× 10 mL), washed with brine (20 mL) dried over sodium sulphate and the organic layer was concentrated in vacuum. The residue was subjected to silica gel column chromatography (gradient elution, 40-50% ethyl acetate/hexane containing 1% AcOH) to furnish N-benzyl-4-nitro-3′-(1H-tetrazol-5-yl)-[1,1′-biphenyl]-3-amine, TRV 1120 as a yellow solid (120 mg, 0.32 mmol), 42% yield. ¹H NMR (500 MHz, DMSO-d₆) δ(ppm) 4.77 (d, J=6 Hz, 2H), 7.07 (dd, J=9 Hz, 2 Hz, 1H), 7.25 (m, 2H), 7.37 (m, 2H), 7.46 (d, J=7 Hz, 2H), 7.71 (t, J=8 Hz, 1H), 7.80 (m, 1H), 8.10 (m, 1H), 8.23 (d, J=8.5 Hz, 1H), 8.28 (m, 1H), 8.78 (t, J=6 Hz, 1H).

### Example 50

### Synthesis of TRV 1126

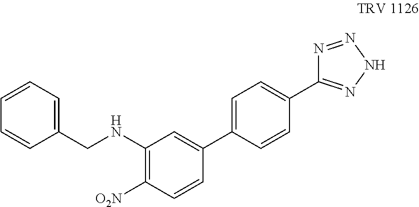 [SMILES: O=[N+]([O-])c1ccc(-c2ccc(-c3nn[nH]n3)cc2)cc1NCc1ccccc1]

A solution of 3′-(benzylamino)-4′-nitro-[1,1′-biphenyl]-4-carbonitrile (250 mg, 0.76 mmol), sodium azide (59 mg, 0.91 mmol) and zinc bromide (503 mg, 2.28 mmol) in NMP was heated to 140° C. for 16 h. The solution was cooled, diluted with HCl (1M, 20 mL) and water (10 mL). The mixture was extracted with ethyl acetate (3× 10 mL), washed with brine (20 mL) dried over sodium sulphate and the organic layer was concentrated in vacuum. The residue was subjected to silica gel column chromatography (gradient elution, 40-50% ethyl acetate/hexane containing 1% AcOH) to furnish N-benzyl-4-nitro-4′-(2H-tetrazol-5-yl)-[1,1′-biphenyl]-3-amine, TRV 1126 as a yellow solid (100 mg, 0.27 mmol), 35% yield. ¹H NMR (500 MHz, DMSO-d₆) δ(ppm) 4.77 (d, J=6 Hz, 2H), 7.07 (dd, J=9 Hz, 2 Hz, 1H), 7.22 (d, J=2 Hz, 1H), 7.27 (t, J=7.7 Hz, 1H), 7.38 (m, 2H), 7.45 (d, J=7 Hz, 2H), 7.87 (m, 2H), 8.13 (m, 2H), 8.20 (d, J=9 Hz, 1H), 8.78 (t, J=6 Hz, 1H).

### Example 51

### Synthesis of TRV 1125

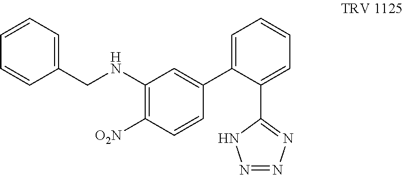 [SMILES: O=[N+]([O-])c1ccc(-c2ccccc2-c2nnn[nH]2)cc1NCc1ccccc1]

A solution of 3′-(benzylamino)-4′-nitro-[1,1′-biphenyl]-2-carbonitrile (250 mg, 0.76 mmol), sodium azide (59 mg, 0.91 mmol) and zinc bromide (503 mg, 2.28 mmol) in NMP was heated to 140° C. for 16 h. The solution was cooled, diluted with HCl (1M, 20 mL) and water (10 mL). The mixture was extracted with ethyl acetate (3×10 mL), washed with brine (20 mL) dried over sodium sulphate and the organic layer was concentrated in vacuum. The residue was subjected to silica gel column chromatography (gradient elution, 40-50% ethyl acetate/hexane containing 1% AcOH) to furnish N-benzyl-4-nitro-2′-(1H-tetrazol-5-yl)-[1,1′-biphenyl]-3-amine, TRV 1125 as a yellow solid (140 mg, 0.38 mmol), 49% yield. ¹H NMR (500 MHz, DMSO-d₆) δ(ppm) 4.45 (d, J=5.5 Hz, 2H), 6.36 (d, J=8.5 Hz, 1H), 6.65 (s, 1H), 7.25-7.36 (m, 5H), 7.43 (d, J=7.5 Hz, 1H), 7.55-7.69 (m, 2H), 7.71 (d, J=7.5 Hz, 1H), 8.00 (d, J=9 Hz, 1H), 8.04 (m, 1H), 8.64 (t, J=5 Hz, 1H).

### Example 52

### Synthesis of TRV 1121

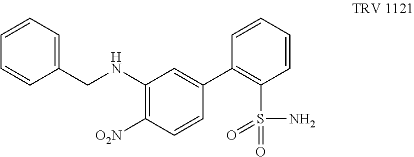 [SMILES: NS(=O)(=O)c1ccccc1-c1ccc([N+](=O)[O-])c(NCc2ccccc2)c1]

To a degassed solution of N-benzyl-2-nitro-5-(4,4,5,5-tetramethyl-1,3,2-dioxaborolan-2-yl)aniline (250 mg, 0.71 mmol), 2-bromobenzenesulfonamide (198 mg, 0.85 mmol) in DME (5 mL) was added 1 mL of sodium carbonate solution (2M) followed by tetrakis(triphenylphosphine) palladium (41 mg, 0.035 mmol). The reaction was vigorously stirred and heated to 90° C. under an atmosphere of argon for 16 h then cooled to room temperature. The mixture was diluted with H₂O (10 mL) and extracted with ethyl acetate (3×10 mL), washed with brine (15 mL), dried (Na₂SO₄) and concentrated in vacuum. The residue was subjected to column chromatography (gradient elution, 40%-70% ethyl acetate/hexanes) to give furnish the title compound, 3′-(benzylamino)-4′-nitro-[1,1′-biphenyl]-2-sulfonamide, TRV 1121 as an orange solid (130 mg, 0.47 mmol) 47% yield. ¹H NMR (500 MHz, DMSO-d₆) δ(ppm) 4.62 (d, J=4.5 Hz, 2H), 6.98 (s, 1H), 7.21-7.61 (m, 8H), 7.82 (m, 1H), 8.01 (m, 2H), 8.09 (m, 1H), 7.85 (m, 1H), 8.67 (bs, 1H).

### Example 53

### Synthesis of TRV 1122

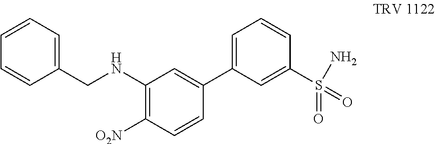 [SMILES: NS(=O)(=O)c1cccc(-c2ccc([N+](=O)[O-])c(NCc3ccccc3)c2)c1]

To a degassed solution of N-benzyl-2-nitro-5-(4,4,5,5-tetramethyl-1,3,2-dioxaborolan-2-yl)aniline (250 mg, 0.71 mmol), 3-bromobenzenesulfonamide (198 mg, 0.85 mmol) in DME (5 mL) was added 1 mL of sodium carbonate solution (2M) followed by tetrakis(triphenylphosphine) palladium (41 mg, 0.035 mmol). The reaction was vigorously stirred and heated to 90° C. under an atmosphere of argon for 16 h then cooled to rt. The mixture was diluted with H₂O (10 mL) and extracted with ethyl acetate (3×10 mL), washed with brine (15 mL), dried (Na₂SO₄) and concentrated in vacuum. The residue was subjected to column chromatography (gradient elution, 40%-70% ethyl acetate/hexanes) to give furnish the title compound 3′-(benzylamino)-4′-nitro-[1,1′-biphenyl]-3-sulfonamide, TRV 1122 as an orange solid (95 mg, 0.23 mmol) 33% yield. ¹H NMR (500 MHz, DMSO-d₆) δ(ppm) 4.75 (d, J=6 Hz, 2H), 7.01 (dd, J=9 Hz, 2 Hz, 1H), 7.18 (d, J=2 Hz, 1H), 7.26 (t, J=7.5 Hz, 1H), 7.32 (m, 6H), 7.68 (t, J=8 Hz, 1H), 7.85 (m, 1H), 8.06 (t, J=1.5 Hz, 1H), 8.23 (d, J=8.5 Hz, 1H), 8.28 (m, 1H), 8.79 (t, J=6 Hz, 1H).

### Example 54

### Synthesis of TRV 1096 and TRV 1097

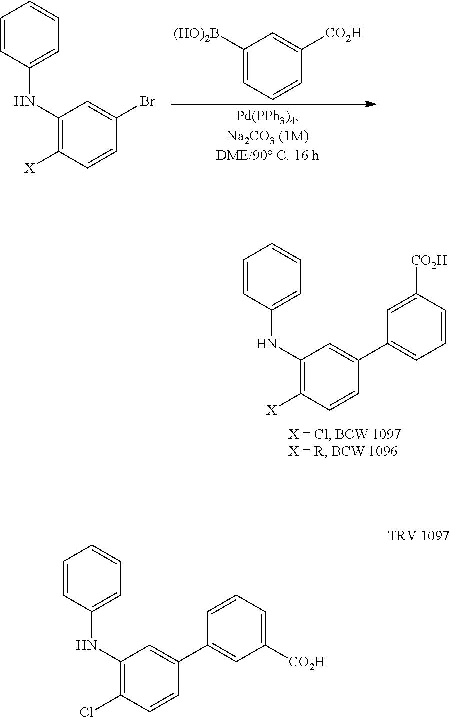

To a degassed solution of 5-bromo-2-chloro-N-phenylaniline (250 mg, 0.87 mmol), 3-boronobenzoic acid (203 mg, 1.20 mmol) and sodium carbonate (2.3 mL, 2M) in DME was charged Pd (PPh₃)₄ (55 mg, 0.048 mmol). The reaction was vigorously stirred and heated to 90° C. under an atmosphere of argon for 16 h then cooled to room temperature. The mixture was diluted with H₂O (10 mL) and extracted with ethyl acetate (3× 10 mL), washed with brine (15 mL), dried (Na₂SO₄) and concentrated in vacuum. The residue was subjected to column chromatography (90% ethyl acetate/hexane, 1% AcOH) to furnish the title compound 4′-chloro-3′-(phenylamino)-[1,1′-biphenyl]-3-carboxylic acid, TRV 1097 (105 mg, 0.325) 34%. ¹H NMR (500 MHz, DMSO-d₆) δ(ppm); 6.93 (t, J 7 Hz, 1H), 7.15 (d, J=7.5 Hz, 2H), 7.22-7.30 (m, 3H), 7.49-7.59 (m, 3H), 7.82 (m, 2H), 7.93 (m, 1H), 8.08 (bs, 1H), 13.41 (bs, 1H).

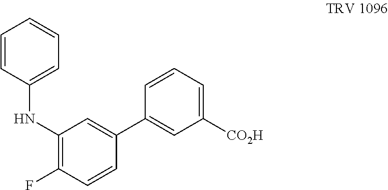 [SMILES: O=C(O)c1cccc(-c2ccc(F)c(Nc3ccccc3)c2)c1]

To a degassed solution of 5-bromo-2-fluoro-N-phenylaniline (250 mg, 0.94 mmol), 3-boronobenzoic acid (203 mg, 1.20 mmol) and sodium carbonate (2.3 mL, 2M) in DME was charged Pd (PPh₃)₄ (55 mg, 0.048 mmol). The reaction was vigorously stirred and heated to 90° C. under an atmosphere of argon for 16 h then cooled to room temperature. The mixture was diluted with H₂O (10 mL) and extracted with ethyl acetate (3×10 mL), washed with brine (15 mL), dried (Na₂SO₄) and concentrated in vacuum. The residue was subjected to column chromatography (90% ethyl acetate/hexane, 1% AcOH) to furnish the title compound 4′-chloro-3′-(phenylamino)-[1,1′-biphenyl]-3-carboxylic acid, TRV 1096 (152 mg, 0.49 mmol) 53%. ¹H NMR (500 MHz, DMSO-d₆) δ(ppm); 6.87 (m, 1H), 7.07 (d, J=7.5 Hz, 2H), 7.24-7.35 (m, 4H), 7.49-7.62 (m, 2H), 7.83 (d, J=7.5 Hz, 1H), 7.92 (d, J=7.5 Hz, 1H), 8.08 (d, J=10 Hz, 2H), 13.09 (bs, 1H).

### Example 55

### Synthesis of TRV 1094 and TRV 1095

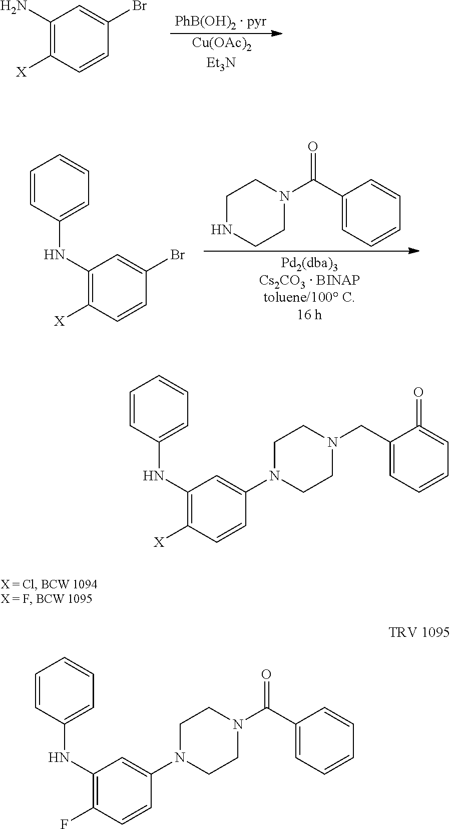

To a degassed solution of 5-bromo-2-fluoro-N-phenylaniline (500 mg, 1.89 mmol), phenyl(piperazin-1-yl)methanonein, (538 mg, 2.83 mmol) Cs₂CO₃ (1.22 g, 3.78 mmol), BINAP (55.9 mg, 0.09 mmol) in toluene was charged Pd₂(dba)₃ (86.4 mg, 0.09 mmol). The reaction was sealed under an atmosphere of argon and heated to 100° C. for 7 h. The mixture was cooled, filtered through celite, washed with ethyl acetate and then concentrated in vacuum. The residue was subjected to silica gel column chromatography (60% hexane/ethyl acetate) to furnish the title compound (4-(4-fluoro-3-(phenylamino)phenyl)piperazin-1-yl)(phenyl)methanone, TRV 1095 as an off white solid (387 mg, 1.03 mmol) 55%. ¹H NMR (500 MHz, CDCl₃) δ(ppm) 2.90-4.00 (m, 8H), 5.77 (bs, 1H), 6.39 (m, 1H), 6.88 (m, 1H), 6.97-7.01 (m, 2H), 7.12 (m, 2H), 7.30 (m, 2H), 7.40-7.50 (m, 5H).

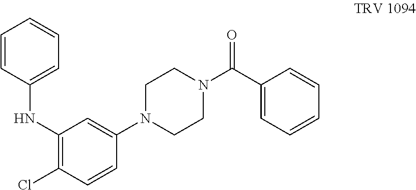 [SMILES: O=C(c1ccccc1)N1CCN(c2ccc(Cl)c(Nc3ccccc3)c2)CC1]

To a degassed solution of 5-bromo-2-chloro-N-phenylaniline (172 mg, 0.61 mmol), phenyl(piperazin-1-yl)methanonein, (139 mg, 0.73 mmol), Cs₂CO₃ (396 mg, 1.22 mmol), BINAP (18.9 mg, 0.03 mmol) in toluene was charged Pd₂(dba)₃ (27.9 mg, 0.03 mmol). The reaction was scaled under an atmosphere of argon and heated to 100° C. for 7 h. The mixture was cooled, filtered through celite, washed with ethyl acetate and then concentrated in vacuum. The residue was subjected to silica gel column chromatography (60% hexane/ethyl acetate) to furnish the title compound (4-(4-chloro-3-(phenylamino)phenyl)piperazin-1-yl)(phenyl)methanone, TRV 1094 as a colorless solid (168 mg, 0.43 mmol) 70%. ¹H NMR (500 MHz, CDCl₃) δ(ppm) 3.02-3.95 (m, 8H), 6.09 (bs, 1H), 6.45 (m, 1H), 6.86 (d, J=2 Hz, 1H), 7.08 (t, J=7.5 Hz, 1H), 7.19 (m, 2H), 7.24 (d, J=8.5 Hz, 1H), 7.36 (m, 2H), 7.40-7.50 (m, 5H).

### Example 56

### Synthesis of TRV 1246

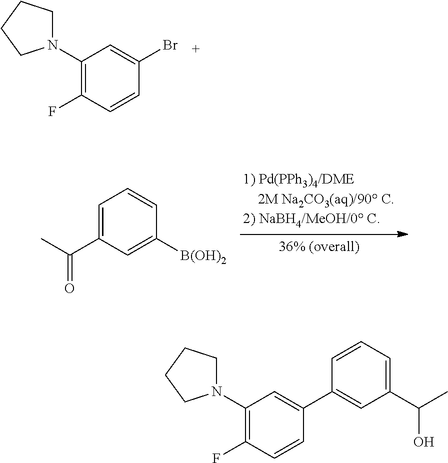 [SMILES: Fc1ccc(Br)cc1N1CCCC1 ; CC(=O)c1cccc(B(O)O)c1 ; CC(O)c1cccc(-c2ccc(F)c(N3CCCC3)c2)c1]

Following the general procedure for a two-step Suzuki coupling—NaBH₄ reduction sequence, 1.50 mmol of 1-bromo-4-fluoro-5-(pyrrolidin-N-yl)benzene was converted to 0.1562 g (36% overall yield) of TRV 1246. ¹H NMR (700 MHz, CDCl3) δ=7.55 (s, 1H), 7.45 (d, J=7.7 Hz, 1H), 7.340 (t, J=7.7 Hz, 1H), 7.34 (d, J=7.7 Hz, 1H), 7.03 (dd, J=13.3, 8.4 Hz, 1H), 6.85 (s, 1H), 6.84 (d, J=8.4 Hz, 1H), 4.97 (q, J=6.3 Hz, 1H), 3.45 (s, 4H), 1.99 (s, 4H), 1.83 (s, 1H), 1.54 (d, J=6.3 Hz, 3H).

### Example 57

### Synthesis of TRV 1248

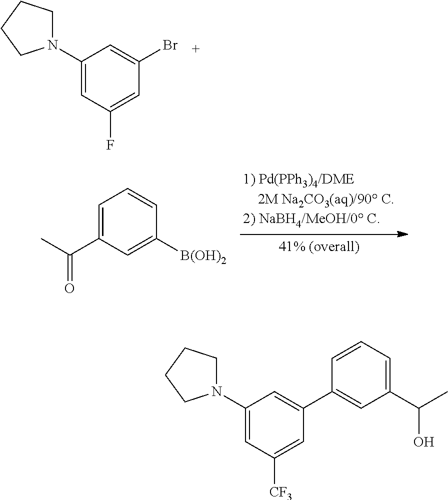 [SMILES: Fc1cc(Br)cc(N2CCCC2)c1 ; CC(=O)c1cccc(B(O)O)c1 ; CC(O)c1cccc(-c2cc(N3CCCC3)cc(C(F)(F)F)c2)c1]

Following the general procedure for a two-step Suzuki coupling—NaBH₄ reduction sequence, 2.02 mmol of 1-bromo-3-fluoro-5-(pyrrolidin-N-yl)benzene was converted to 0.5261 g (87.9% overall yield) of TRV 1248. ¹H NMR (700 MHz, CDCl3) δ=7.57 (s, 1H), 7.50 (d, J=7.7 Hz, 1H), 7.47 (d, J=7.7 Hz, 1H), 7.43 (t, J=7.37 Hz, 1H), 7.40 (d, J=7.7 Hz, 1H), 6.87 (d, J=8.4 Hz, 1H), 6.80 (s, 1H), 5.00-4.97 (m, 1H), 3.68-3.66 (m, 4H), 2.04-2.02 (m, 4H), 1.86 (d, J=3.5 Hz, 1H), 1.54 (d, J=6.3 Hz, 3H).

### Example 58

### In Vivo Efficacy Trials in the 5xFAD Transgenic AD Mouse Model

The 5xFAD mouse (B6SJL-Tg(SwFLon,PSEN1*M 146L*L286V)6799Vas/J; JAX #006554) overexpresses human APP(695) with the Swedish (K670N,M671L), Florida (I716V) and London (V717I) familial AD (FAD) mutations, as well as human presenilin 1 (PSI) with two FAD mutations (M 146L and L286V). These five mutations act additively, leading to large and rapid age-related increases in neuronal Aβ-40 and Aβ-42 peptides beginning at 2 months of age. The 5xFAD mouse shows an earlier onset and more rapid development of neuropathology than other mouse models of AD. Extra-cellular Aβ plaques are first observed in the hippocampus, subiculum, frontal cortex and spinal cord at 2-3 months of age and increase with age. No neurofibrillary tangles (tau) are observed in the brains of 5xFAD mice. Neuroinflammation has also been found in the 5xFAD mouse, which shows age-dependent increases in active astrocytes and microglia as early as 2 months of age, and by 9 months of age extensive gliosis is present in the hippocampus and cortex. Active astrocytes are commonly found surrounding β-amyloid plaques and levels of the pre-synaptic marker synaptophysin are decreased, indicating a decrease in synaptic connectivity to the 5xFAD mouse (Oakley et al., 2006). The 5xFAD mouse shows impaired long-term synaptic plasticity of CA1 neurons at 6 months of age. Age-related cognitive impairment in visuo-spatial working memory in the Y-maze occurs at 4-5 months of age. The 5xFAD mice also show a short-term memory deficit in the novel object recognition test after a 60-minute delay at 8-9 months of age; visuo-spatial learning and memory deficits in the Morris water maze (MWM) at 4-6 months of age; and in contextual fear conditioning at 6-7 months of age.

The general procedure for an in vivo efficacy trial with 5xFAD mice is as follows. Compound administration begins at 2 months of age. The arms for an efficacy trial include 5xFAD with compound, 5xFAD with vehicle, WT with compound and WT with vehicle. Activity levels, anxiety, spatial reference memory and spatial working memory are tested in 5xFAD mice and their wildtype (WT) littermate controls at 6 months. After behavioral testing, mice are sacrificed and their blood and brains assessed for Aβ levels.

Results to date with compound TRV 1140 indicate that there was no difference in behavioural performance between WT with compound and WT with vehicle, suggesting the compound does not have effects on behaviour in WT mice. 5xFAD mice treated with a daily i.p. 30 mg/kg dose of TRV 1140 had behavioural performance similar to that of vehicle-treated WT mice in reference and working memory tests, while vehicle-treated 5xFAD performed worse. These results indicated that treatment with TRV 1140 has beneficial effects on cognitive impairment in the 5xFAD model of AD.

The invention claimed is:

1. A compound of any of Formulas I: 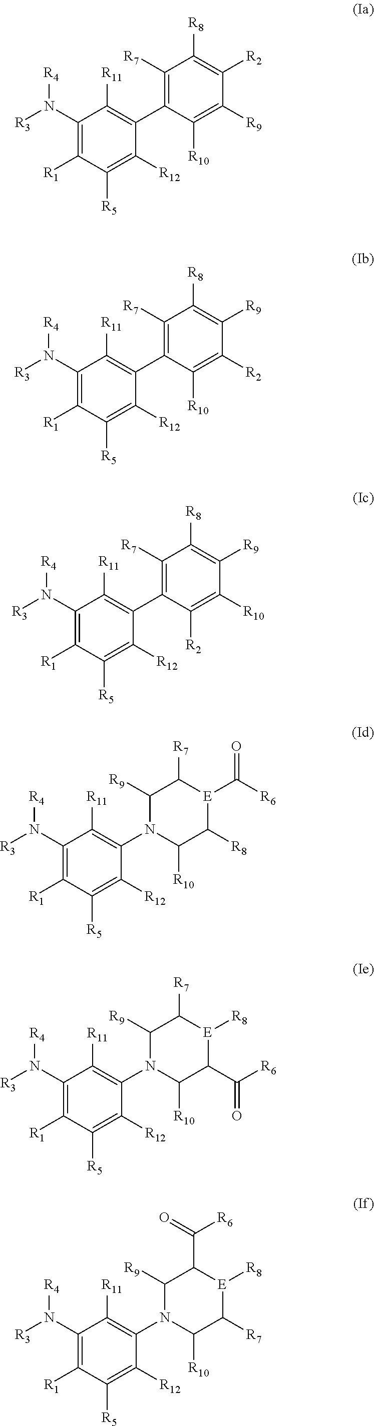
in which R₁ is selected from the group consisting of nitro, difluoromethyl ketone, halogen, trifluoromethylsulfone, trifluoromethyl ether, difluoromethyl ether, hydrogen, trifluoromethyl, cyano, isopropylamine, and N-linked tetrazole;
R_{2,} when present, is selected from the group consisting of C-linked tetrazole, sulfonamide, alkylamide, dialkylamide, benzyl alkylamide, N-pyrrolidinamide, (N′-methanonylpiperazine)amide, (N′-methylpiperazine)amide, morphilinamide, piperidineamide, ethanol-1-yl, methanol, 2,2,2-trifluoro-1-hydroxyethanol-1-yl, 2,2,2-trifluoroethanol-1-yl, and cyano;
R₃ is selected from the group consisting of benzyl, isopropyl, ethyl, cyclopropyl, and cyclobutyl;
R₄ is selected from the group consisting of hydrogen, CH₂CH₂NCH₃, alkyl, 3-(N-pyrrolidinyl)propyl, propyl, and CH₂CH₂O;
R₃ and R₄ are unconnected or connect to form piperizine, N-pyrrolidine, 4-(N-pyrrolidinyl)piperidine, morpoline, or piperidine rings;
R₅ is selected from the group consisting of hydrogen, halogen, and trifluoromethyl;
R₆, when present, is selected from the group consisting of alkyl, N-pyrollidine, alkylamine, dialkylamine, and phenyl optionally substituted with alkyl, halogen, or alkoxy independently at each open position;
E, when present, is selected from the group consisting of carbon and nitrogen;
and R₇, R₈, R₉, R₁₀, R₁₁, and R₁₂ are each independently selected from the group consisting of hydrogen and alkyl.

2. The compound of claim 1 in which the compound is according to Formula Ia.

3. The compound of claim 1 in which the compound is according to Formula Ib.

4. The compound of claim 1 in which the compound is according to Formula Ic.

5. The compound of claim 1 in which the compound is according to Formula Id.

6. The compound of claim 1 in which the compound is according to Formula Ie.

7. The compound of claim 1 in which the compound is according to Formula If.

8. The compound of claim 1 in which E is carbon.

9. The compound of claim 1 in which E is nitrogen.

10. The compound of claim 3 in which R₁ is nitro and R₂ is selected from the group consisting of ethanol-1-yl, methanol, 2,2,2-trifluoro-1-hydroxyethanol-1-yl, and 2,2,2-trifluoroethanol-1-yl.

11. The compound of claim 10 in which the compound is 1-(3′-(benzylamino)-4′-nitro-[1,1′-biphenyl]-3-yl)-2,2,2-trifluoroethanol.

12. The compound of claim 10 in which the compound is 1-(3′-(benzylamino)-4′-nitro-[1,1′-biphenyl]-3-yl)-2,2,2-trifluoroethane-1,1-diol.

13. The compound of claim 3 in which R₁ is nitro and R₂ is C-linked tetrazole.

14. The compound of claim 6 in which R₁ is nitro.

15. The compound of claim 14 in which R₂ is selected from the group consisting of sulfonamide, alkylamide, dialkylamide, benzyl alkylamide, N-pyrrolidinamide, (N′-methanonylpiperazine)amide, (N′-methylpiperazine)amide, morphilinamide, and piperidineamide.

16. The compound of claim 1, wherein compound inhibits the aggregation of an amyloidogenic protein.

17. The compound of claim 1 for use in the preparation of a pharmaceutically effective dosage form the treatment of amyloid diseases.

18. The compound of claim 16, wherein said amyloid disease is selected from the group consisting of Alzheimer's disease, Parkinson's disease, Huntington's disease, and prion diseases.

19. A pharmaceutical composition, comprising a compound of claim 1 and a pharmaceutically acceptable excipient.

20. The composition of claim 19, wherein said composition is an oral dosage form.

21. The composition of claim 19, wherein said composition is a parenteral dosage form.

22. A compound selected from the group consisting of those compounds identified herein by TRV 1093 to TRV 1248 inclusive.

23. A method of treatment for an amyloid disease comprising administering a therapeutically effective dose of a compound of claim 1 to a subject in need thereof.
